(19) United States
(12) Patent Application Publication
(10) Pub. No.: US 2023/0022546 A1
(43) Pub. Date: Jan. 26, 2023

(54) METHODS AND SYSTEMS FOR SLOWING BRAIN ATROPHY

(71) Applicant: Cognito Therapeutics Inc., Cambridge, MA (US)

(72) Inventors: Zach Malchano, Cambridge, MA (US); Evan Hempel, Cambridge, MA (US); Colleen Cotter, Cambridge, MA (US); Aylin Cimenser, Cambridge, MA (US); Alyssa Boasso, Cambridge, MA (US); Holly Mrozak, Cambridge, MA (US); Alex Konisky, Cambridge, MA (US); Nathan Strozewski, Cambridge, MA (US); Taylor Travers, Cambridge, MA (US); Martin Williams, Cambridge, MA (US); Kim Kwan, Cambridge, MA (US); Brent Vaughan, Cambridge, MA (US); Tom Megerian, Cambridge, MA (US); Mihály Hagós, Cambridge, MA (US)

(21) Appl. No.: 17/300,314

(22) Filed: Mar. 8, 2022

Related U.S. Application Data
(63) Continuation of application No. PCT/US2022/019370, filed on Mar. 8, 2022.
(60) Provisional application No. 63/158,779, filed on Mar. 9, 2021.
(60) Provisional application No. 63/244,522, filed on Sep. 15, 2021.

(51) Int. Cl. A61N 1/36

(52) U.S. Cl. CPC A61N 1/36025

(57) ABSTRACT

Systems and methods of the present disclosure are directed to neural stimulation via non-invasive sensory stimuli. The non-invasive sensory stimuli can reduce neuroinflammation, improving synaptic plasticity and stimulating neural networking, and improving microglial-mediated clearance of cerebral insults, which would otherwise contribute to the progression of brain atrophy, by inducing synchronized gamma oscillations in at least one region of a brain in a subject. Stimulations can adjust, control or otherwise manage the frequency of the neural oscillations to provide beneficial effects to one or more cognitive states or cognitive functions of the brain, while mitigating or preventing adverse consequences on a cognitive state or cognitive function that stem from progression of brain atrophy.

### CROSS-REFERENCE

This application is a continuation of International Application No. PCT/US2022/019370, filed Mar. 8, 2022, which claims the benefit of U.S. Provisional Patent Application No. 63/158,779, filed Mar. 9, 2021, and U.S. Provisional Patent Application No. 63/244,522, filed Sep. 15, 2021, each of which is incorporated herein by reference in its entirety.

### BACKGROUND

Neural oscillation occurs in humans or animals and includes rhythmic or repetitive neural activity in the central nervous system. Neural tissue can generate oscillatory activity by mechanisms within individual neurons or by interactions between neurons. Oscillations can appear as either oscillations in membrane potential or as rhythmic patterns of action potentials, which can produce oscillatory activation of post-synaptic neurons. Synchronized activity of a group of neurons can give rise to macroscopic oscillations, which can be observed by electroencephalography (“EEG”). Neural oscillations can be characterized by their frequency, amplitude and phase. Neural oscillations can give rise to electrical impulses that form a brainwave. These signal properties can be observed from neural recordings using time-frequency analysis.

### INCORPORATION BY REFERENCE

Each patent, publication, and non-patent literature cited in the application is hereby incorporated by reference in its entirety as if each was incorporated by reference individually.

### SUMMARY

In some embodiments, disclosed herein is a method for reducing a rate of brain atrophy in one or more areas of the brain of a subject, the method comprising administering a non-invasive stimulus to the subject to induce synchronized gamma oscillations in at least one region of the brain of the subject, thereby reducing the rate of brain atrophy in one or more areas of the brain of the subject.

In some embodiments, the non-invasive stimulus comprises one or more stimulation waveforms.

The one or more stimulation waveforms may comprise visual stimulation waveforms, auditory stimulation waveforms, haptic stimulation waveforms, mechanical stimulation waveforms, or a combination thereof.

In some embodiments, the one or more stimulation waveforms have synchronous phases. In some embodiments, the one or more stimulation waveforms comprise a first stimulation waveform and a second stimulation waveform. In some cases, the first stimulation waveform comprises a visual stimulation waveform. In some cases, the first stimulation waveform comprises an auditory stimulation waveform. In some cases, the first stimulation waveform comprises a mechanical stimulation waveform. In some cases, the first stimulation waveform comprises a vibrotactile or haptic stimulation waveform.

In some embodiments, the second stimulation waveform comprises an auditory stimulation waveform. In some embodiments, the second stimulation waveform comprises a mechanical stimulation waveform. In some embodiments, the second stimulation waveform comprises a vibrotactile or haptic stimulation waveform. In some embodiments, the first stimulation waveform comprises a square wave function. In some embodiments, the first stimulation waveform comprises a sinusoidal wave function. In some embodiments, the second stimulation waveform comprises a square wave function. In some embodiments, the second stimulation waveform comprises a sinusoidal wave function.

In some embodiments, administering the non-invasive stimulus comprises administering the non-invasive stimulus for a first time duration. In some cases, the method further comprises measuring a response of the subject to the non-invasive stimulus during a second time duration. In some cases, the first time duration and the second time duration are separated by a third time duration.

In many embodiments described herein, the non-invasive stimulus is delivered through a wearable device. In some cases, the wearable device comprises glasses. In some cases, the glasses comprise an illumination source. In some cases, the glasses comprise opaque glasses. In some cases, the glasses comprise transparent glasses. In some cases, the wearable device further comprises headphones.

In some embodiments, methods and systems described herein further comprise measuring a response of the subject to the non-invasive stimulus. In some cases, measuring the response occurs during the first duration. In some cases, measuring the response occurs during the second duration. In some cases, measuring the response occurs during the first duration. In some cases, measuring the response occurs during the third duration. In some cases, measuring the response occurs during the first duration and the second duration. In some cases, measuring the response occurs during the third duration.

The one or more areas of the brain may comprise a visual cortex, a somatosensory cortex, an insular cortex, or any combination thereof. In some embodiments, reducing the rate of brain atrophy comprises reducing a rate of decrease in brain volume. In some cases, the rate of decrease in brain volume comprises from about 0.3 cm³ per month to about 2 cm³ per month. In some cases, the rate of decrease in brain volume comprises from about 0.3 cm³ per year to about 2 cm³ per year.

In some cases, the rate of decrease in brain volume comprises a rate of decrease in hippocampal volume, lateral lobe volume, lateral ventricle volume, temporal lobe volume, occipital lobe volume, temporal cortex thickness, occipital cortex thickness, or a combination thereof.

The present disclosure further provides systems and methods for treating a condition, disorder, or disease associated with brain atrophy in a subject, the method comprising administering a non-invasive stimulus to the subject to generate synchronized gamma oscillations in at least one brain region, wherein the administering reduces a rate of brain atrophy experienced by the subject, thereby treating the condition, disorder, or disease associated with brain atrophy in the subject. In some cases, the condition, disorder, or disease comprises a microglial-mediated disease. In some cases, the condition, disorder, or disease comprises a neurodegenerative disorder. In some cases, the neurodegenerative disorder comprises Alzheimer's disease, Creutzfeldt-Jakob disease (CJD), variant CJD, Gerstmann-Sträussler Scheinker Syndrome, Fatal Familial Insomnia, Kuru, or any combination thereof. In some cases, the condition, disorder, or disease comprises aging.

In some embodiments described herein, the rate of brain atrophy is reduced from a first rate to a second rate, wherein the first rate comprises at least about 0.5% of brain atrophy per year, and the second rate comprises less than the first rate. In some cases, the first rate comprises at least 0.6% of brain atrophy per year. In some cases, the first rate comprises at least 0.7% of brain atrophy per year. In some cases, the first rate comprises at least 0.8% of brain atrophy per year. In some cases, the first rate comprises at least 0.9% of brain atrophy per year. In some cases, the first rate comprises at least 1.0% of brain atrophy per year. In some cases, the first rate comprises at least 1.1% of brain atrophy per year. In some cases, the first rate comprises at least 1.2% of brain atrophy per year. In some cases, the first rate comprises at least 1.3% of brain atrophy per year. In some cases, the first rate comprises at least 2.0% of brain atrophy per year. In some cases, the first rate comprises at least 3.0% of brain atrophy per year. In some cases, the first rate comprises at least 4.0% of brain atrophy per year.

Further provided herein are methods and systems for mitigating cognitive decline linked to atrophy of a brain, the method comprising administering a non-invasive stimulus to a subject in need thereof to induce synchronized gamma oscillations in at least one region of the brain, wherein the administering causes a reduction in a rate at which the brain experiences atrophy, thereby mitigating cognitive decline linked to atrophy of the brain.

Also disclosed herein are systems and methods of reducing one or more symptoms or conditions associated with brain atrophy, the method comprising: (a) identifying a subject experiencing brain atrophy; and (b) administering non-invasive stimulus to a subject, the non-invasive sensory stimulus causing synchronization of one or more brainwaves, thereby reducing the one or more symptoms associated with brain atrophy. In some cases, the one or more symptoms or conditions comprise loss of neurons, memory loss, blurred vision, aphasia, impaired balance, paralysis, decreases in cortical volume, increases in CSF volume, loss of motor control, difficulty speaking, difficulty with reading comprehension, decrease in gray matter volume, decreases in white matter volume, decrease in neuronal size, loss of neuronal cytoplasmic proteins, or any combination thereof. In some embodiments, identifying the subject comprises evaluating a condition of the subject, performing an assessment of the subject, or measuring neuronal activity of the subject.

In some embodiments, systems and methods disclosed herein further comprise evaluating a response of the subject to the non-invasive sensory stimulus. In some embodiments, the systems and methods further comprise adjusting the non-invasive sensory stimulus to enhance the synchronization.

In some embodiments, the non-invasive stimulus comprises a frequency from about 20 Hz to about 70 Hz. In some embodiments, the non-invasive stimulus comprises a frequency from about 30 Hz to about 60 Hz. In some embodiments, the non-invasive stimulus comprises a frequency of from about 35 Hz to about 45 Hz.

In some embodiments, adjusting comprises alternating the non-invasive sensory stimulus from a square-wave function to a sinusoidal wave function. In some cases, adjusting comprises alternating the non-invasive sensory stimulus from a square-wave function to a sinusoidal wave function. In some cases, adjusting comprises adjusting intensity of the non-invasive stimulus. In some cases, adjusting comprises adjusting frequency of the non-invasive stimulus. In some cases, adjusting comprises a waveform of the non-invasive stimulus. In some cases, adjusting comprises altering a source of the non-invasive sensory stimulus.

Also provided herein is a non-transitory computer-readable storage media encoded with instructions executable by one or more processors, wherein the instructions implement any one of the systems and methods described above. Further provided is a computer-implemented system comprising: at least one digital processing device comprising at least one processor and instructions executable by the at least one processor, wherein the instructions implement any one of the methods described herein.

The present disclosure further provides a system for reducing a rate of brain atrophy in a subject comprising: a) a stimulus-emitting component capable of providing a neural stimulus, an auditory stimulus, or a visual stimulus to a subject; b) a processor; c) a memory device; and d) a feedback sensor, wherein the processor: (i) receives an indication of a physiological assessment, a cognitive assessment, a neural assessment, a physical assessment, or any combination thereof, of the subject through the feedback sensor; and (ii) instructs the stimulus-emitting component based on the indication to adjust at least one parameter associated with the neural stimulus, the auditory stimulus, or the visual stimulus to create an improvement in the degree of neural entrainment exhibited by neurons in at least one brain region of the subject, thereby causing a reduction in the rate of brain atrophy.

### BRIEF DESCRIPTION OF THE DRAWINGS

FIG. 1 illustrates a block diagram depicting a system to perform neural stimulation via visual stimulation in accordance with an embodiment.

FIG. 2A-2F illustrate visual stimulation signals that cause neural stimulation in accordance with some embodiments.

FIG. 3A-3C illustrate fields of vision in which visual signals can be transmitted for visual brain entrainment in accordance with some embodiments.

FIG. 4A-4C illustrate devices configured to transmit visual signals for neural stimulation in accordance with some embodiments.

FIG. 5A-5D illustrate devices configured to transmit visual signals for neural stimulation in accordance with some embodiments.

FIG. 6A and FIG. 6B illustrate devices configured to receive feedback to facilitate neural stimulation in accordance with some embodiments.

FIG. 7A and FIG. 7B are block diagrams depicting embodiments of computing devices useful in connection with the systems and methods described herein.

FIG. 8 is a flow diagram of a method of performing neural stimulation using visual stimulation in accordance with an embodiment.

FIG. 9 is a block diagram depicting a system for neural stimulation via auditory stimulation in accordance with an embodiment.

FIG. 10A-10I illustrate audio signals and types of modulations to audio signals used to induce neural oscillations via auditory stimulation in accordance with some embodiments.

FIG. 11A illustrates audio signals generated using binaural beats, in accordance with an embodiment.

FIG. 11B illustrates acoustic pulses having isochronic tones, in accordance with an embodiment.

FIG. 11C illustrates audio signals having a modulation technique including audio filters, in accordance with an embodiment.

FIG. 12A-12C illustrate configurations of systems for neural stimulation via auditory stimulation in accordance with some embodiments.

FIG. 13 illustrates a configuration for a system for room-based auditory stimulation for neural stimulation in accordance with an embodiment.

FIG. 14 illustrates devices configured to receive feedback to facilitate neural stimulation via auditory stimulation in accordance with some embodiments.

FIG. 15 is a flow diagram of a method of performing auditory brain entrainment in accordance with an embodiment.

FIG. 16 is a block diagram depicting a system for neural stimulation via multiple modes of stimulation in accordance with an embodiment.

FIG. 17A is a block diagram depicting a system for neural stimulation via visual stimulation and auditory stimulation in accordance with an embodiment.

FIG. 17B is a diagram depicting waveforms used for neural stimulation via visual stimulation and auditory stimulation in accordance with an embodiment.

FIG. 18 is a flow diagram of a method for neural stimulation via visual stimulation and auditory stimulation in accordance with an embodiment.

FIG. 19 is an efficacy summary chart for the modified intent to treat (mITT) population, including p-values, difference, confidence intervals (CI), and a standardized estimate of efficacy based on the values.

FIG. 20 shows the separate means analysis, on the left, and the linear model analysis, on the right, of the Alzheimer's Disease composite score (ADCOMS) as optimized for mid and moderate Alzheimer's Disease (MADCOMS) for the sham and active treatment groups.

FIG. 21 shows the separate means analysis, on the left, and a linear model analysis, on the right, of the Alzheimer's Disease Assessment Scale-Cognitive Subscale 14 (ADAS-Cog14) values for the sham and active treatment groups.

FIG. 22 shows the separate means analysis, on the left, and a linear model analysis, on the right, of the Clinical Dementia Rating Sale Sum of Boxes (CDR-SB) values for the sham and active treatment groups.

FIG. 23 shows the separate means analysis, on the left, and a linear model analysis, on the right, of the Alzheimer's Disease Cooperative Study—Activities of Daily Living Scale (ADCS-ADL) scores for the sham and active treatment groups.

FIG. 24 shows the linear model analysis of the Mini-Mental State Examination (MMSE) score, as measured after six months of treatment (i.e., at the last time point).

FIG. 25 shows the linear model analysis of magnetic resonance imaging (MRI) results of whole brain volume value, on the left, and hippocampal volume, on the right, after six months of treatment.

FIG. 26 is a table depicting a summary of efficacy findings resulting from the human clinical trial, including p-values, treatment differences, CI values and the percentage of slowing of brain atrophy.

FIG. 27A and FIG. 27B summarize the findings of the ITT population primary analysis. FIG. 27A shows the summary findings for the ITT population primary analysis of occipital cortical volume change in cm³ from the human clinical trial, including the least-square mean change from baseline (±SE) and p-values at 0, 3, and 6 months for both sham and active treatment arms. FIG. 27B shows the summary findings for the ITT population primary analysis of occipital cortex thickness in mm from the human clinical trial, including the least-square mean change from baseline (±SE) and p-values at 0, 3, and 6 months for both sham and active treatment arms.

The features and advantages of the present solution will become more apparent from the detailed description set forth below when taken in conjunction with the drawings, in which like reference characters identify corresponding elements throughout. In the drawings, like reference numbers generally indicate like elements.

### DETAILED DESCRIPTION

### Using Neural Stimulation to Slow Brain Atrophy and Mitigate Related Conditions

Described herein are systems and methods for using sensory-evoked potentials to slow brain atrophy and, as a result, mediate symptoms of brain atrophy in a subject. In particular, the present disclosure describes systems and methods for reducing neuroinflammation, improving synaptic plasticity and stimulating neural networking, and improving microglial-mediated clearance of cerebral insults, which otherwise would contribute to the progression of brain atrophy, by inducing synchronized gamma oscillations in at least one region of a brain in a subject. The at least one brain region, for example, can include a visual cortex, a somatosensory cortex, an insular cortex, and/or a hippocampus of the subject.

### Brain Atrophy and Volume

Certain ranges are presented herein with numerical values being preceded by the term “about.” “About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Brain atrophy may present as a change in brain volume. Brain atrophy, or atrophy of brain tissue, describes the loss of volume within neurons, extracellular space, or glia. Atrophy may occur at different rates in different areas or regions of the brain, and it may be reflected by changes in whole brain volume. For an adult, whole brain volume can be, for example, between around 950 cm³ and 1550 cm³. For an adult female, average whole brain volume can be around 1130 cm³. For an adult male, average brain volume can be around 1260 cm³. For a child of an age between around 4 years old and 16 years old, whole brain volume can be, for example, between about 60 cm³ and about 120 cm³.

The present disclosure provides systems and methods directed to slowing a change in brain volume relating to brain atrophy. The change in brain volume can be a reduction of around: 0.3 cm³ per month, 0.5 cm³ per month, 1 cm³ per month, 2 cm³ per month, 0.3 cm³ per year, 0.5 cm³ per year, 1 cm³ per year, 2 cm³ per year, 3 cm³ per year, 4 cm³ per year, 5 cm³ per year, 6 cm³ per year, 7 cm³ per year, 8 cm³ per year, 9 cm³ per year, 10 cm³ per year, 11 cm³ per year, 12 cm³ per year, 13 cm³ per year, 14 cm³ per year, or 15 cm³ per year or 16 cm³ per year. The rate of brain atrophy can differ between individuals. Exemplary rates of brain atrophy can include, but are not limited to, rates around: between 0.1% and 0.5% per year, between 0.5% and 1.5% per year, between 1.0% and 3.0% per year, or between 3.0% and 6.0% per year. The rate of brain atrophy can vary based on the cause of atrophy. For example, a healthy individual can experience an average brain atrophy rate of 0.1% and 0.4% per year. In contrast, for subjects with Multiple Sclerosis (MS), the average brain atrophy rate can be between 0.5% and 1.3% per year. The average rate of whole brain atrophy for a patient with Alzheimer's Disease can be, for example, between 1.0% and 4.0% per year. Aging can also cause brain atrophy rates to increase. For example, an individual in their mid-thirties can experience a rate of brain atrophy that is around 0.2% per year, and an individual at around age sixty can experience a rate of brain atrophy that is around 0.5% per year.

Brain volume can be measured using magnetic resonance imaging (MRI) or computerized tomography (CT) scans. Loss of brain volume may be measured by comparing brain volume over time. Various methods can be used to measure brain volume or changes in brain volume, which indicate brain atrophy. Most commonly, brain volume or brain volume loss can be measured using cross-sectional methods or longitudinal methods. Cross-sectional methods can use a single MRI scan to segment particular tissues or structures and calculate the volume of these tissue types and/or structures. Longitudinal methods can use at least two MRI scans of the same subject at different points in time to calculate brain volume changes or atrophy. Longitudinal methods may seek to match the two MRI scans using warping techniques and, from this process, directly extract small changes in brain volume.

Various tools and algorithms may be employed to determine brain volume through CT or MRI scans. Of the various toolkits available for determining brain volume and changes in brain volume based on the scanned images, examples include, but are not limited to, the following tools: Atropos, an opensource tissue segmentation algorithm; CIVET, a web-based image-processing tool for volumetric analysis with different human brain images; the Structural Image Evaluation using Normalization of Atrophy (SIENA and SIENAX), a software that applies a Brain Extraction Tool (BET) to determine cross-sectional volumes; MSmetrix, a fully-automatic tool that detects brain lesions and calculates lesion volume and measures whole-brain and gray matter atrophy; and Statistical Parametric Mapping (SPM), which for analysis of images in a MATLAB environment.

### Sources of Brain Atrophy

Systems and methods described herein may alleviate brain atrophy, or symptoms thereof, resulting from normal aging of a subject. The systems and methods described herein may also alleviate brain atrophy, or symptoms thereof, resulting from a disease, disorder, or condition. Additionally, systems and methods of the present disclosure may help slow progression of diseases, disorders, or conditions associated with brain atrophy, and their associated symptoms, through non-invasive stimulation of gamma oscillations.

Systems and methods of the present disclosure may slow brain atrophy and alleviate symptoms of brain atrophy through non-invasive stimulation of gamma oscillations. A subject with brain atrophy may experience this as a cause or result of a variety of conditions, disorders, or diseases, including but not limited to: normal aging, Alzheimer's Disease (AD), dementia, Parkinson's disease, seizure, cerebral palsy, senile dementia, pick's disease, Huntington's disease, Krabbe disease, leukodystrophies, multiple sclerosis, epilepsy, anorexia nervosa, aphasia, learning disability, frontotemporal dementia, expressive aphasia, receptive aphasia, Lewy body dementia, chronic traumatic encephalopathy (CTE), and others.

The systems and methods of the present disclosure are also directed to alleviating symptoms of brain atrophy. Systems and methods described herein may alleviate brain atrophy, or symptoms thereof, resulting from normal aging of a subject. The systems and methods described herein may also alleviate brain atrophy, or symptoms thereof, resulting from a disease, disorder, or condition. Additionally, systems and methods of the present disclosure may help slow progression of diseases, disorders, or conditions associated with brain atrophy, and their associated symptoms, through non-invasive stimulation of gamma oscillations.

Symptoms may include a loss of neurons, memory loss, blurred vision, aphasia, impaired balance, paralysis, decreases in cortical volume, increases in CSF volume, loss of motor control, difficulty speaking, comprehension, reading comprehension, memory, decrease in gray and/or white matter, decrease in neuronal size, loss of neuronal cytoplasmic proteins, or any combination thereof. In some embodiments, the present disclosure describes systems and methods which act to slow the onset of symptoms of brain atrophy. The present disclosure provides systems and methods for treating any of the above-listed diseases and disorders by reducing any of the above-listed symptoms associated with brain atrophy.

Systems and methods of the present disclosure may slow brain atrophy and alleviate symptoms of brain atrophy through non-invasive stimulation of gamma oscillations. A subject with brain atrophy may experience this as a cause or result of a variety of conditions, disorders, or diseases, including but not limited to: normal aging, Alzheimer's Disease (AD), dementia, Parkinson's disease, seizure, cerebral palsy, senile dementia, pick's disease, Huntington's disease, Krabbe disease, leukodystrophies, multiple sclerosis, epilepsy, anorexia nervosa, aphasia, learning disability, frontotemporal dementia, expressive aphasia, receptive aphasia, Lewy body dementia, chronic traumatic encephalopathy (CTE), and others.

Accordingly, in some embodiments, the present disclosure provides systems and methods for alleviating symptoms associated with a microglial-mediated disease or disorder associated with brain atrophy. For example, the microglial-mediated disease or disorder may comprise a neurodegenerative disease associated with tauopathy, including but not limited to chronic traumatic encephalopathy, frontotemporal dementia, and corticobasilar degeneration. The microglial-mediated disease or disorder may comprise a genetic disorder, such as an inherited ataxia associated with brain atrophy. The microglial-mediated disease or disorder may also comprise a neuropsychiatric disorder associated with brain atrophy, such as depression or schizophrenia; brain injury, such as stroke; or demyelinating diseases, such as Multiple Sclerosis and Acute disseminated encephalomyelitis.

### Neurodegenerative Diseases Causing Tauopathy: Alzheimer's Disease, Frontotemporal Dementia, Chronic Traumatic Encephalopathy, and Corticobasilar Degeneration

In some embodiments, the microglial-mediated disease or disorder may comprise a neurodegenerative disease associated with tauopathy, including but not limited to Alzheimer's disease, frontotemporal dementia, chronic traumatic encephalopathy (CTE), and corticobasilar degeneration.

Alzheimer's disease (AD) is a progressive neurodegenerative disease characterized by a decline in memory, orientation, and reasoning. AD may be characterized by the accumulation of amyloid plaques comprising the amyloid-β (Aβ) peptide and neurofibrillary tangles (NFTs) made of the tau protein. Under normal conditions, the soluble Aβ peptide is produced and secreted by neurons and subsequently cleared from the brain via cerebral spinal fluid (CSF) pathways. However, in subjects with AD, the Aβ peptide appears to aggregate into higher-order species to form soluble oligomers and insoluble plaques in a concentration-dependent manner. This aggregation may initiate many neurotoxic events including disrupted brain metabolism, neuroinflammation, reduced functional connectivity, synaptic and neuronal loss, and/or formation of NFTs.

Frontotemporal dementia (FTD) is a group of disorders that result from damage to the frontal and temporal lobes of the brain. Depending on the location of the damage, the disorder causes changes in social behavior, personality, and/or loss of language skills. In some people, FTD may also lead to neuromuscular disorder, such as parkinsonism. Frontotemporal dementia occurs where abnormal proteins build up in the brain, leading to death of brain cells and atrophy of the frontal and temporal lobes of the brain. Frontotemporal dementia occurs in Alzheimer's disease, although it may be caused by other neurodegenerative diseases as well.

Chronic traumatic encephalopathy (CTE) is characterized by symptoms that may include memory loss, confusion, impaired judgment, impulse control problems, aggression, depression, anxiety, suicidality, parkinsonism, and progressive dementia. CTE results from traumatic injury to the head triggers microglia, leading to tau proteins becoming phosphorylated at progressively higher rates and, accordingly, accumulation of hyperphosphorylated tau deposits. The buildup of phosphorylated tau proteins can lead to axonal transport defects, neuroinflammation, and synapse loss.

Corticobasal degeneration (CBD) is characterized by cell loss and deterioration of specific areas of the brain. In corticobasal degeneration, abnormal levels of tau accumulate in certain brain cells, eventually causing their deterioration. Symptoms often initially include experiencing motor abnormalities in one limb that progressively spreads to all limbs. Such motor abnormalities include, for example, progressive stiffening or tightening of muscles in the limb (progressive asymmetric rigidity) and the inability to perform purposeful or voluntary movements (apraxia). Trouble with speech and language, including aphasia, apraxia of speech, dysarthria, dysphagia. Symptoms may also be reflected in physical movements and tremors, such as experiencing action tremor, postural tremor, bradykinesia, akinesia, myoclonus, and ataxic gait. The severity and type of symptoms depend on the area of the brain affected by the disease, which is most commonly the cerebral cortex and basal ganglia.

### Genetic Disorders: Inherited Ataxias.

As stated above, the present systems and methods may be used to alleviate symptoms associated with inherited ataxias. Hereditary ataxias are characterized by slowly progressive incoordination of gait and are often associated with poor coordination of hands, speech, and eye movements. Hereditary ataxias frequently cause atrophy of the cerebellum as a result of impaired circuitry and function of the cerebellar cortex, a result of neurodegeneration of cellular afferents and the Purkinje cells, which have long axonal projections that comprise the only sources of output from the cerebellar cortex to deep cerebellar nuclei.

Neuropsychiatric Disorders: Schizophrenia, Depression, Chronic Stress

In other embodiments, the present disclosure provides system and methods for treating neuropsychiatric disorders associated with brain atrophy, which is mediated by microglial cells. For example, individuals with schizophrenia often show reduced postmortem cortical tissue. This phenomenon is caused by synaptic pruning, which reflects abnormalities in microglia-like cells and synaptic function. In other embodiments, the present disclosure provides methods and systems for alleviating symptoms of depression. Stress, impaired neurogenesis, and defects in synaptic plasticity are associated with depression. Chronic stress promotes microglial hyper-ramification and astroglial atrophy. Thus, in some embodiments, the system and methods disclosed may alleviate symptoms associated with chronic stress or depression by improving synaptic plasticity and stimulating neural networking, along with improving microglial-mediated clearance.

### Brain Injury: Stroke and Related Cerebrovascular Diseases

In some embodiments, the present disclosure provides systems and methods for alleviating symptoms associated with a stroke. For example, the stroke may be an ischemic stroke, which causes a neuroinflammatory response and activates microglia to help repair the brain. Ischemic stroke is associated with disappearance of synaptic activity. As a result, brain tissue within the penumbra during an ischemic stroke is structurally intact, but functionally silent. Failure to reperfuse this penumbral region or resupply glucose and oxygen in time may lead to atrophy of brain cells located in the penumbra. In contrast, activating synapses in this region may delay cell death and salvage brain tissue. By improving synaptic plasticity and stimulating neural networking, the present systems and methods can reduce brain atrophy and related symptoms associated with ischemic stroke. Other forms cerebrovascular diseases with similar symptoms—e.g., neuroimmune modulation, synaptic function—may also be treated by the present disclosure, including but not limited to: transient ischemic attack (TIA), hemorrhagic stroke, arteriovenous malformation, intracranial atherosclerosis (ICAD), and Moyamoya.

### Demyelinating Diseases: Multiple Sclerosis and Acute Disseminated Encephalomyelitis

In some embodiments, the present disclosure provides systems and methods for alleviating symptoms of demyelinating diseases associated with brain atrophy. For example, the demyelinating disease may comprise Multiple Sclerosis or Acute disseminated encephalomyelitis, both of which may cause neuroinflammation and cerebral atrophy. In multiple sclerosis (MS), brain or cerebral atrophy is common due to demyelination and destruction of nerve cells. Widespread myelin damage occurs, causing damage to the myelin-rich white matter of the brain, occurs as a result of a number of attacks which occur over time. In acute disseminated encephalomyelitis, similar symptoms are seen, but the onset of widespread myelin damage is often due to a single episode or attack. By reducing neuroinflammation and stimulating neural networking, the present disclosure provides systems and methods for slowing brain atrophy associated with demyelinating diseases and related symptoms.

### Transmissible Spongiform Encephalopathies (Prion Diseases)

In some embodiments, the present disclosure provides systems and methods for alleviating symptoms of Prion diseases associated with brain atrophy. Prion diseases, also known as transmissible spongiform encephalopathies, comprise a group of fatal neurodegenerative diseases which include, for example, Creutzfeldt-Jakob Disease (CJD), Variant Creutzfeldt-Jakob Disease (vCJD), Gerstmann-Sträussler-Scheinker Syndrome, Fatal Familial Insomnia, Kuru, and others. In some cases, prion diseases can have similar symptoms with other and/or with AD. In some cases, different types of prion diseases can cause brain damage that exhibit similar features such as: extensive spongiform degeneration, widespread neuronal loss, synaptic alterations, atypical brain inflammation, and accumulation of protein aggregates. In some cases, prion diseases such as CJD, Kuru, and Gerstmann-Sträussler-Scheinker disease, may form amyloid plaques that is similar to those observed in AD.

### Delivery Methods and Systems

The present disclosure provides methods directed at reducing progression of brain atrophy by evoking gamma wave oscillations in a subject, the method comprising delivering a gamma oscillation inducing waveform and/or evoking gamma wave oscillations in a subject. In some cases, the gamma oscillation inducing waveform is provided as a visual signal. In some embodiments, the gamma oscillation inducing waveform induces a signal that is indicative of an expected outcome, the outcome relating to a symptom or condition associated with brain atrophy.

Provided herein are systems and methods for reducing a rate of brain atrophy in one or more areas of the brain of a subject, the method comprising administering a non-invasive stimulus to the subject to induce synchronized gamma oscillations in at least one region of the brain of the subject, thereby reducing the rate of brain atrophy in one or more areas of the brain of the subject. In some embodiments, the non-invasive stimulus comprises one or more stimulation waveforms. The one or more stimulation waveforms may comprise visual stimulation waveforms, auditory stimulation waveforms, haptic stimulation waveforms, mechanical stimulation waveforms, or a combination thereof.

In some embodiments, the one or more stimulation waveforms have synchronous phases. In some embodiments, the one or more stimulation waveforms comprise a first stimulation waveform and a second stimulation waveform. In some cases, the first stimulation waveform comprises a visual stimulation waveform. In some cases, the first stimulation waveform comprises an auditory stimulation waveform. In some cases, the first stimulation waveform comprises a mechanical stimulation waveform. In some cases, the first stimulation waveform comprises a vibrotactile or haptic stimulation waveform.

In some embodiments, the second stimulation waveform comprises an auditory stimulation waveform. In some embodiments, the second stimulation waveform comprises a mechanical stimulation waveform. In some embodiments, the second stimulation waveform comprises a vibrotactile or haptic stimulation waveform. In some embodiments, the first stimulation waveform comprises a square wave function. In some embodiments, the first stimulation waveform comprises a sinusoidal wave function. In some embodiments, the second stimulation waveform comprises a square wave function. In some embodiments, the second stimulation waveform comprises a sinusoidal wave function.

In some embodiments, administering the non-invasive stimulus comprises administering the non-invasive stimulus for a first time duration. In some cases, the method further comprises measuring a response of the subject to the non-invasive stimulus during a second time duration. In some cases, the first time duration and the second time duration are separated by a third time duration.

The systems and methods provided herein may provide gamma oscillation inducing waveform through various sources of stimulation. For example, in some embodiments, the gamma oscillation inducing waveform is delivered through one or more of: visual stimulation, auditory stimulation, tactile or haptic stimulation, olfactory stimulation, or bone conduction.

In some embodiments, the gamma oscillation inducing waveform is delivered at least in part through one or more devices in the user's environment. For example, in some cases, the gamma oscillation inducing waveform is delivered at least in part through one or more of a speaker, lighting fixtures, bed attachment, wall mounted screen, or other household device. In some embodiments, the one or more devices are controlled by a further device, such as a phone, tablet, or home automation hub, configured to manage the delivery of the gamma oscillation inducing waveform through the one or more devices in the user's environment. In some embodiments, the gamma oscillation inducing waveform is delivered through more than one device in the user's environment.

In some embodiments, the gamma oscillation inducing waveform is delivered to more than one subject present in a space. In an exemplary embodiment, the gamma oscillation inducing waveform is delivered to more than one subject in a space through one or more devices present in the space. In some cases, the one or more devices deliver the same stimulus to all subjects present in the space. In some cases, the one or more devices deliver the same stimulus to a subset of the subjects present in the space. In some cases, the one or more devices deliver a stimulus customized to an individual subject.

In some embodiments, the gamma oscillation inducing waveform is delivered via an article of clothing or body attachment worn by a subject. In some cases, the gamma oscillation inducing waveform is delivered at least in part through glasses, goggles, a mask, or other worn apparatus that provide stimulation.

In some embodiments, the gamma oscillation inducing waveform is delivered through vibrotactile or haptic (touch) stimulation. For example, the gamma oscillation inducing waveform may be delivered via a device that vibrates at a frequency sufficient for entrainment of gamma oscillations. The vibrotactile stimulation may be delivered via an article of clothing or a body attachment.

In some embodiments, the gamma oscillation inducing waveform provides a visual signal. In some cases, the visual signal is delivered through a light source, such as a light bulb or LED screen. In some embodiments, the gamma oscillation inducing waveform comprises visual stimulation, provided as a visual signal, which is delivered through a pair of glasses worn by the subject. In some embodiments, the gamma oscillation inducing waveform is delivered through a pair of opaque or partially transparent glasses. Such glasses may contain illuminating elements on the interior or other sources of light which provide the visual signal. In some cases, the wearable device further comprises headphones. In some embodiments, the waveform is provided as an audio signal. In some embodiments, the gamma oscillation inducing waveform is delivered via an audio source, such as a pair of headphones, a speaker, or earphones. In some embodiments, the gamma oscillation inducing waveform is delivered at least in part through headphones or earbuds. For example, in some embodiments visual and auditory signals are provided by such headphones and glasses worn together at the same time. In some embodiments, the combined visual and auditory signals are delivered via one device.

In some embodiments, methods and systems described herein further comprise measuring a response of the subject to the non-invasive stimulus. In some cases, measuring the response occurs during the first duration. In some cases, measuring the response occurs during the second duration. In some cases, measuring the response occurs during the first duration. In some cases, measuring the response occurs during the third duration. In some cases, measuring the response occurs during the first duration and the second duration. In some cases, measuring the response occurs during the third duration.

The one or more areas of the brain may comprise a visual cortex, a somatosensory cortex, an insular cortex, or any combination thereof. In some embodiments, reducing the rate of brain atrophy comprises reducing a rate of decrease in brain volume. In some cases, the rate of decrease in brain volume comprises from about 0.3 cm³ per month to about 2 cm³ per month. In some cases, the rate of decrease in brain volume comprises from about 0.3 cm³ per year to about 2 cm³ per year.

In some cases, the rate of decrease in brain volume comprises a rate of decrease in hippocampal volume, lateral lobe volume, lateral ventricle volume, temporal lobe volume, occipital lobe volume, temporal cortex thickness, occipital cortex thickness, or a combination thereof.

The present disclosure further provides systems and methods for treating a condition, disorder, or disease associated with brain atrophy in a subject, the method comprising administering a non-invasive stimulus to the subject to generate synchronized gamma oscillations in at least one brain region, wherein the administering reduces a rate of brain atrophy experienced by the subject, thereby treating the condition, disorder, or disease associated with brain atrophy in the subject. In some cases, the condition, disorder, or disease comprises a microglial-mediated disease. In some cases, the condition, disorder, or disease comprises a neurodegenerative disorder. In some cases, the neurodegenerative disorder comprises Alzheimer's disease, Creutzfeldt-Jakob disease (CJD), variant CJD, Gerstmann-Sträussler Scheinker Syndrome, Fatal Familial Insomnia, Kuru, or any combination thereof. In some cases, the condition, disorder, or disease comprises aging.

In some embodiments described herein, the rate of brain atrophy is reduced from a first rate to a second rate, wherein the first rate comprises at least about 0.5% of brain atrophy per year, and the second rate comprises less than the first rate. In some cases, the first rate comprises at least 0.6% of brain atrophy per year. In some cases, the first rate comprises at least 0.7% of brain atrophy per year. In some cases, the first rate comprises at least 0.8% of brain atrophy per year. In some cases, the first rate comprises at least 0.9% of brain atrophy per year. In some cases, the first rate comprises at least 1.0% of brain atrophy per year. In some cases, the first rate comprises at least 1.1% of brain atrophy per year. In some cases, the first rate comprises at least 1.2% of brain atrophy per year. In some cases, the first rate comprises at least 1.3% of brain atrophy per year. In some cases, the first rate comprises at least 2.0% of brain atrophy per year. In some cases, the first rate comprises at least 3.0% of brain atrophy per year. In some cases, the first rate comprises at least 4.0% of brain atrophy per year.

Further provided herein are methods and systems for mitigating cognitive decline linked to atrophy of a brain, the method comprising administering a non-invasive stimulus to a subject in need thereof to induce synchronized gamma oscillations in at least one region of the brain, wherein the administering causes a reduction in a rate at which the brain experiences atrophy, thereby mitigating cognitive decline linked to atrophy of the brain.

Also disclosed herein are systems and methods of reducing one or more symptoms or conditions associated with brain atrophy, the method comprising: (a) identifying a subject experiencing brain atrophy; and (b) administering non-invasive stimulus to a subject, the non-invasive sensory stimulus causing synchronization of one or more brainwaves, thereby reducing the one or more symptoms associated with brain atrophy. In some cases, the one or more symptoms or conditions comprise loss of neurons, memory loss, blurred vision, aphasia, impaired balance, paralysis, decreases in cortical volume, increases in CSF volume, loss of motor control, difficulty speaking, difficulty with reading comprehension, decrease in gray matter volume, decreases in white matter volume, decrease in neuronal size, loss of neuronal cytoplasmic proteins, or any combination thereof. In some embodiments, identifying the subject comprises evaluating a condition of the subject, performing an assessment of the subject, or measuring neuronal activity of the subject.

In some embodiments, systems and methods disclosed herein further comprise evaluating a response of the subject to the non-invasive sensory stimulus. In some embodiments, the systems and methods further comprise adjusting the non-invasive sensory stimulus to enhance the synchronization.

In some embodiments, the non-invasive stimulus comprises a frequency from about 20 Hz to about 70 Hz. In some embodiments, the non-invasive stimulus comprises a frequency from about 30 Hz to about 60 Hz. In some embodiments, the non-invasive stimulus comprises a frequency of from about 35 Hz to about 45 Hz.

In some embodiments, adjusting comprises alternating the non-invasive sensory stimulus from a square-wave function to a sinusoidal wave function. In some cases, adjusting comprises alternating the non-invasive sensory stimulus from a square-wave function to a sinusoidal wave function. In some cases, adjusting comprises adjusting intensity of the non-invasive stimulus. In some cases, adjusting comprises adjusting frequency of the non-invasive stimulus. In some cases, adjusting comprises a waveform of the non-invasive stimulus. In some cases, adjusting comprises altering a source of the non-invasive sensory stimulus.

Also provided herein is a non-transitory computer-readable storage media encoded with instructions executable by one or more processors, wherein the instructions implement any one of the systems and methods described above. Further provided is a computer-implemented system comprising: at least one digital processing device comprising at least one processor and instructions executable by the at least one processor, wherein the instructions implement any one of the methods described herein.

The present disclosure further provides a system for reducing a rate of brain atrophy in a subject comprising: a) a stimulus-emitting component capable of providing a neural stimulus, an auditory stimulus, or a visual stimulus to a subject; b) a processor; c) a memory device; and d) a feedback sensor, wherein the processor: (i) receives an indication of a physiological assessment, a cognitive assessment, a neural assessment, a physical assessment, or any combination thereof, of the subject through the feedback sensor; and (ii) instructs the stimulus-emitting component based on the indication to adjust at least one parameter associated with the neural stimulus, the auditory stimulus, or the visual stimulus to create an improvement in the degree of neural entrainment exhibited by neurons in at least one brain region of the subject, thereby causing a reduction in the rate of brain atrophy.

### Program Parameters and Parameter Values

Systems and methods herein may involve administration of stimulation for a certain duration of time or administration of stimulation at a certain frequency. For example, in some cases, the gamma oscillation inducing waveform is delivered for about 5 minutes, about 10 minutes, about 20 minutes, about 30 minutes, about 40 minutes, about 50 minutes, or about 1 hour. In some embodiments, the gamma oscillation inducing waveform is delivered for less than 30 minutes. In some embodiments, the gamma oscillation inducing waveform is delivered for up to 30 minutes. In some embodiments, the gamma oscillation inducing waveform is delivered for over 30 minutes. In some embodiments, the gamma oscillation inducing waveform is delivered for up to 1 hour. In some embodiments, the gamma oscillation inducing waveform is delivered for between 10 and 20 minutes, between 20 and 40 minutes, or between 30 and 60 minutes. Stimulation may be delivered for a period of up to 1 week. Stimulation may be delivered for a 1 to 3 month period. Stimulation may be delivered for a 3 to 6 month period. Additionally, stimulation may be delivered for a period of time longer than 6 months. Stimulation may be delivered for one or more weeks, for one or more months, or for one or more years.

In some embodiments, stimulation is delivered over an open-ended period of time, such that the subject receiving the gamma oscillation inducing waveform determines the period of time by choosing to receive the stimulation. In some embodiments, the stimulus is delivered in periods of varying durations. For example, the stimulus may be delivered for a first duration and for a second duration. The first duration may be between zero and 5 minutes, between 5 and 10 minutes, between 10 and 20 minutes, between 20 and 30 minutes, between 30 and 40 minutes, between 40 and 50 minutes, or between 50 and 60 minutes. The first duration may be for more than 60 minutes. The second duration may be between zero and 5 minutes, between 5 and 10 minutes, between 10 and 20 minutes, between 20 and 30 minutes, between 30 and 40 minutes, between 40 and 50 minutes, or between 50 and 60 minutes. The second duration may be for more than 60 minutes. The second duration may be the same as the first duration. The second duration may differ than the first duration.

In some cases, the stimulation is provided on an hourly basis. In some cases, stimulation is provided on a daily basis. In some embodiments, stimulation is delivered for multiple periods over the course of a day. In some cases, the stimulation is provided at least one time per week. In some cases, the frequency of stimulation varies for a given time period. In some cases, the duration of stimulation varies for a given time period. In other cases, both the frequency of stimulation and the duration of stimulation varies for a given time period.

The stimulation may be provided, for example, on at least a daily basis, at least a weekly basis, at least a biweekly basis, or at least a monthly basis. In some cases, stimulation is delivered for at least one hour each day. In other cases, stimulation is delivered for at least two hours each day. In some embodiments, stimulation is delivered for less than one hour each day. In some embodiments, stimulation is delivered for at least three hours each day. In some embodiments, stimulation is delivered for more than three hours each day.

In some embodiments, delivery of a gamma oscillation inducing waveform is performed such that a first signal (e.g., a visual signal, an audio signal, or a haptic signal) and a second signal (e.g., a visual signal, an audio signal, or a haptic signal) are offset relative to each other by a delay. In some embodiments, such signals are delivered such that a first signal and a second signal are synchronized. For example, in some embodiments, the first signal has a frequency with a first value, and the second signal has a frequency with a second value. The first value and the second value may differ. The first value and the second value may be substantially the same. In some embodiments, the first signal and the second signal are provided via more than one stimulation source.

In some embodiments, gamma oscillation inducing waveform parameters are configured with a variety of timing and intensity parameters. In some embodiments, these parameters are preconfigured; in some embodiments they are adjusted at least in part by a third party such as a caregiver or healthcare provider; in some embodiments one or more parameters are adjusted responsive to measurements or analysis of one or more of: user context, measured sleep quality related parameters associated with the user, observed, or detected use of the stimulation device. In some embodiments, gamma oscillation inducing waveform are adjusted responsive to detected or analyzed neurogenerative disease symptom progression. Various frequencies and various intensities may be used as parameters for the gamma oscillation inducing waveform.

In some embodiments, one or more stimulus parameters are based at least partially on various clinical measures of treatment outcomes of cognitive function disclosed herein. In some embodiments, varying combinations of stimulus parameters are used during different time periods and subsequent stimulation parameters are selected at least in part based on comparison of clinical measures of treatment outcomes for cognitive function among at least some of those periods.

In some embodiments, the present disclosure delivers 40 Hz non-invasive audio, visual, or combined audio-visual stimulation. In some embodiments stimulus is delivered at one or more stimulation frequencies in the approximate range of 35-45 Hz. In some embodiments, “gamma” refers to frequencies in the range 35-45 Hz.

In some embodiments, specific visual parameters include one or more of: stimulation frequency, intensity (brightness), hue, visual patterns, spatial frequency, contrast, and duty-cycle. In an exemplary embodiment, visual stimulation is provided at a stimulation frequency of about 20 Hz, about 30 Hz, about 40 Hz, about 50 Hz, about 60 Hz, or about 70 Hz. I some embodiments, the stimulation waveforms have a duty-cycle of less than 50%. In some embodiments, the stimulation waveforms have a duty-cycle of more than 50%. In some embodiments, the stimulation waveforms have a duty-cycle of 50%.

In some embodiments, non-invasive stimulation is delivered as combined visual and auditory stimulation, delivered at frequency that provides gamma oscillation inducing waveforms. In some embodiments, visual and auditory stimulation is synchronized to begin each cycle simultaneously. In some embodiments, the beginning of each auditory and visual stimulation cycle is offset by a configured time. In some embodiments, visual and auditory signals are delivered at an intensity clearly recognized by subjects and adjusted to their tolerance level.

In some embodiments specific audio parameters include one or more of: stimulation frequency, intensity (volume), and duty-cycle. In some embodiments, audio frequency is adjusted responsive to a subject's hearing characteristics, for example to frequencies that a subject is better at hearing.

In some embodiments, non-invasive stimulation parameters are directed at inducing gamma wave oscillations in the brains of human subjects. In some embodiments, non-invasive stimulation parameters are directed at inducing alpha waves in human subjects. In some embodiments, the non-invasive stimulation parameters are directed at inducing beta waves in human subjects. In some embodiments, the non-invasive stimulation parameters are directed at inducing gamma waves in human subjects, thereby reducing brain atrophy in the subject.

### Neural Stimulation via Visual Stimulation

Systems and methods of the present disclosure are directed to controlling frequencies of neural oscillations using visual signals and, in doing so, slowing brain atrophy in a subject. The visual stimulation can adjust, control, or otherwise affect the frequency of the neural oscillations to provide beneficial effects to one or more cognitive states or cognitive functions of the brain, or the immune system, while mitigating or preventing adverse consequences on a cognitive state or cognitive function. The visual stimulation can result in brainwave entrainment that can provide beneficial effects to one or more cognitive states of the brain, cognitive functions of the brain, the immune system, or inflammation. In some cases, the visual stimulation can result in local effect, such as in the visual cortex and associate regions. The brainwave entrainment can treat disorders, maladies, diseases, inefficiencies, injuries, or other issues related to a cognitive function of the brain, cognitive state of the brain, the immune system, or inflammation.

Neural oscillation occurs in humans or animals and includes rhythmic or repetitive neural activity in the central nervous system. Neural tissue can generate oscillatory activity by mechanisms within individual neurons or by interactions between neurons. Oscillations can appear as either oscillations in membrane potential or as rhythmic patterns of action potentials, which can produce oscillatory activation of post-synaptic neurons. Synchronized activity of a group of neurons can give rise to macroscopic oscillations, which, for example, can be observed by electroencephalography (“EEG”), magnetoencephalography (“MEG”), functional magnetic resonance imaging (“fMRI”), or electrocorticography (“ECoG”). Neural oscillations can be characterized by their frequency, amplitude and phase. These signal properties can be observed from neural recordings using time-frequency analysis.

For example, an EEG can measure oscillatory activity among a group of neurons, and the measured oscillatory activity can be categorized into frequency bands as follows: delta activity corresponds to a frequency band from 1-4 Hz; theta activity corresponds to a frequency band from 4-8 Hz; alpha activity corresponds to a frequency band from 8-12 Hz; beta activity corresponds to a frequency band from 13-30 Hz; and gamma activity corresponds to a frequency band from 30-70 Hz.

The frequency and presence or activity of neural oscillations can be associated with cognitive states or cognitive functions such as information transfer, perception, motor control and memory. Based on the cognitive state or cognitive function, the frequency of neural oscillations can vary. Further, certain frequencies of neural oscillations can have beneficial effects or adverse consequences on one or more cognitive states or function. However, it may be challenging to synchronize neural oscillations using external stimulus to provide such beneficial effects or reduce or prevent such adverse consequences.

Brainwave entrainment (e.g., neural entrainment or brain entrainment) occurs when an external stimulation of a particular frequency is perceived by the brain and triggers neural activity in the brain that results in neurons oscillating at a frequency corresponding to the particular frequency of the external stimulation. Thus, brain entrainment can refer to synchronizing neural oscillations in the brain using external stimulation such that the neural oscillations occur at a frequency that corresponds to the particular frequency of the external stimulation.

Systems and methods of the present disclosure can provide external visual stimulation to achieve brain entrainment. For example, external signals, such as light pulses or high-contrast visual patterns, can be perceived by the brain. The brain, responsive to observing or perceiving the light pulses, can adjust, manage, or control the frequency of neural oscillations. The light pulses generated at a predetermined frequency and perceived by ocular means via a direct visual field or a peripheral visual field can trigger neural activity in the brain to induce brainwave entrainment. The frequency of neural oscillations can be affected at least in part by the frequency of light pulses. While high-level cognitive function may gate or interfere with some regions being entrained, the brain can react to the visual stimulation at the sensory cortices. Thus, systems and methods of the present disclosure can provide brainwave entrainment using external visual stimulus such as light pulses emitted at a predetermined frequency to synchronize electrical activity among groups of neurons based on the frequency of light pulses. The entrainment of one or more portion or regions of the brain can be observed based on the aggregate frequency of oscillations produced by the synchronous electrical activity in ensembles of cortical neurons. The frequency of the light pulses can cause or adjust this synchronous electrical activity in the ensembles of cortical neurons to oscillate at a frequency corresponding to the frequency of the light pulses.

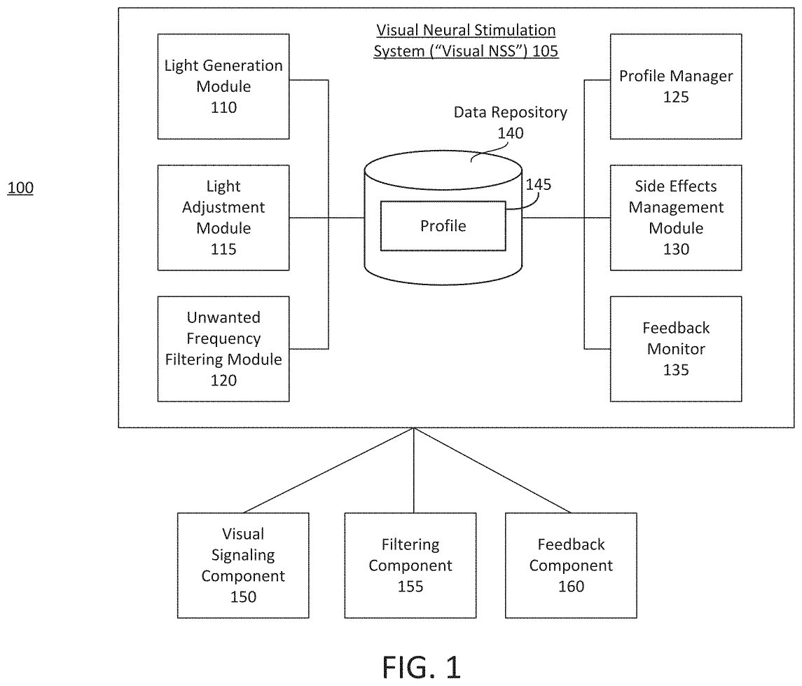

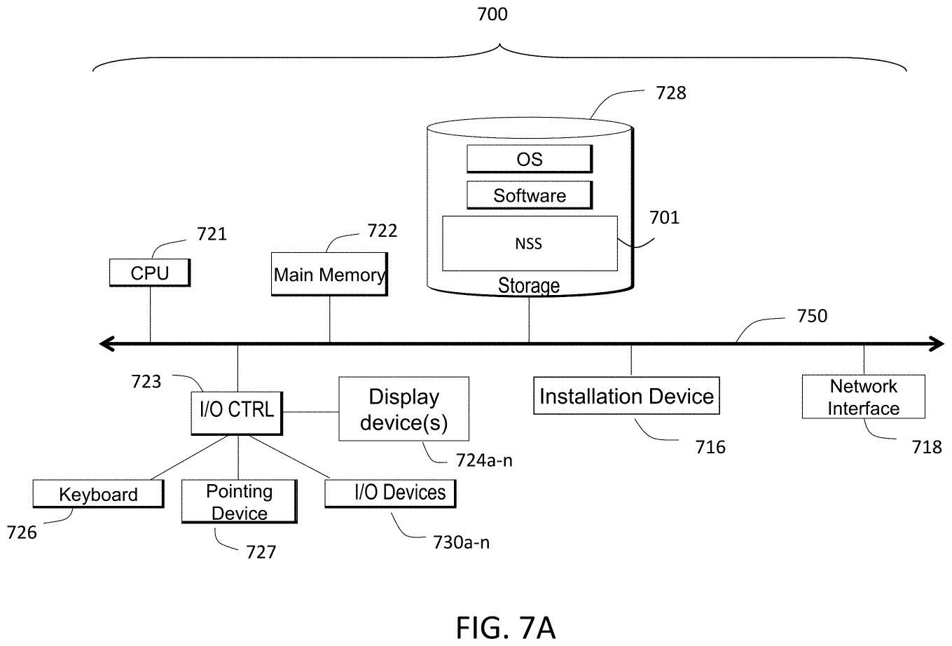

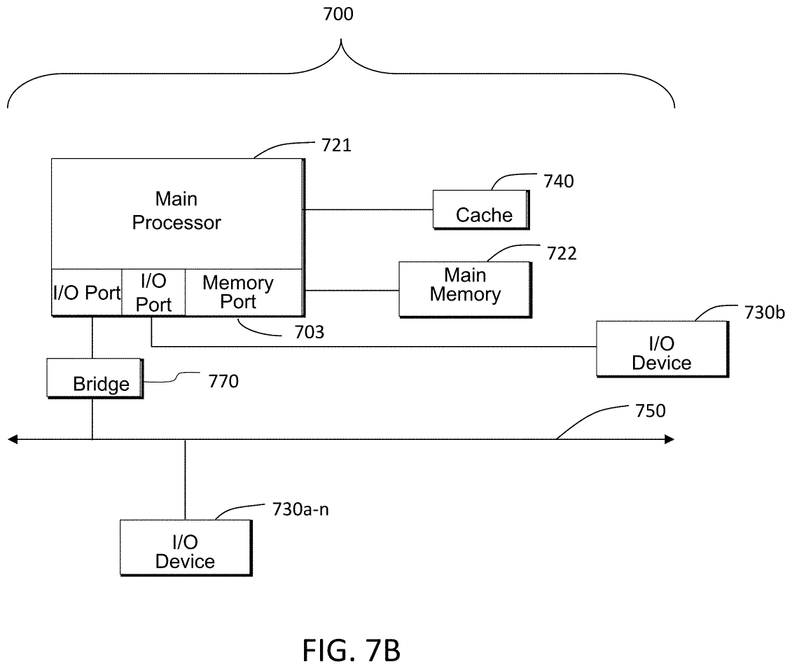

FIG. 1 is a block diagram depicting a system to perform visual brain entrainment in accordance with an embodiment. The system 100 can include a neural stimulation system (“NSS”) 105. The NSS 105 can be referred to as visual NSS 105 or NSS 105. In brief overview, the NSS 105 can include, access, interface with, or otherwise communicate with one or more of a light generation module 110, light adjustment module 115, unwanted frequency filtering module 120, profile manager 125, side effects management module 130, feedback monitor 135, data repository 140, visual signaling component 150, filtering component 155, or feedback component 160. The light generation module 110, light adjustment module 115, unwanted frequency filtering module 120, profile manager 125, side effects management module 130, feedback monitor 135, visual signaling component 150, filtering component 155, or feedback component 160 can each include at least one processing unit or other logic device such as programmable logic array engine, or module configured to communicate with the database repository 150. The light generation module 110, light adjustment module 115, unwanted frequency filtering module 120, profile manager 125, side effects management module 130, feedback monitor 135, visual signaling component 150, filtering component 155, or feedback component 160 can be separate components, a single component, or part of the NSS 105. The system 100 and its components, such as the NSS 105, may include hardware elements, such as one or more processors, logic devices, or circuits. The system 100 and its components, such as the NSS 105, can include one or more hardware or interface component depicted in system 700 in FIGS. 7A and 7B. For example, a component of system 100 can include or execute on one or more processors 721, access storage 728 or memory 722, and communicate via network interface 718.

Still referring to FIG. 1, and in further detail, the NSS 105 can include at least one light generation module 110. The light generation module 110 can be designed and constructed to interface with a visual signaling component 150 to provide instructions or otherwise cause or facilitate the generation of a visual signal, such as a light pulse or flash of light, having one or more predetermined parameter. The light generation module 110 can include hardware or software to receive and process instructions or data packets from one or more module or component of the NSS 105. The light generation module 110 can generate instructions to cause the visual signaling component 150 to generate a visual signal. The light generation module 110 can control or enable the visual signaling component 150 to generate the visual signal having one or more predetermined parameters.

The light generation module 110 can be communicatively coupled to the visual signaling component 150. The light generation module 110 can communicate with the visual signaling component 150 via a circuit, electrical wire, data port, network port, power wire, ground, electrical contacts or pins. The light generation module 110 can wirelessly communicate with the visual signaling component 150 using one or more wireless protocols such as Bluetooth, Bluetooth Low Energy, Zigbee, Z-Wave, IEEE 802.11, WIFI, 3G, 4G, LTE, near field communications (“NFC”), or other short, medium or long range communication protocols, etc. The light generation module 110 can include or access network interface 718 to communicate wirelessly or over a wire with the visual signaling component 150.

The light generation module 110 can interface, control, or otherwise manage various types of visual signaling components 150 in order to cause the visual signaling component 150 to generate, block, control, or otherwise provide the visual signal having one or more predetermined parameters. The light generation module 110 can include a driver configured to drive a light source of the visual signaling component 150. For example, the light source can include a light emitting diode (“LED”), and the light generation module 110 can include an LED driver, chip, microcontroller, operational amplifiers, transistors, resistors, or diodes configured to drive the LED light source by providing electricity or power having certain voltage and current characteristics.

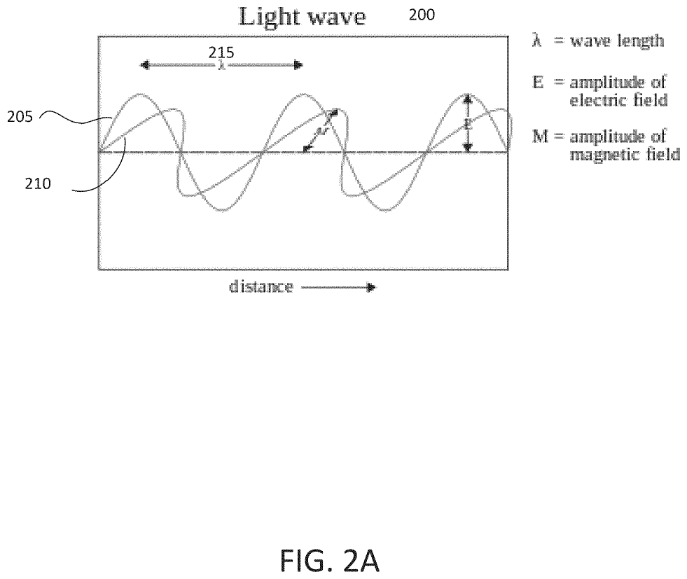

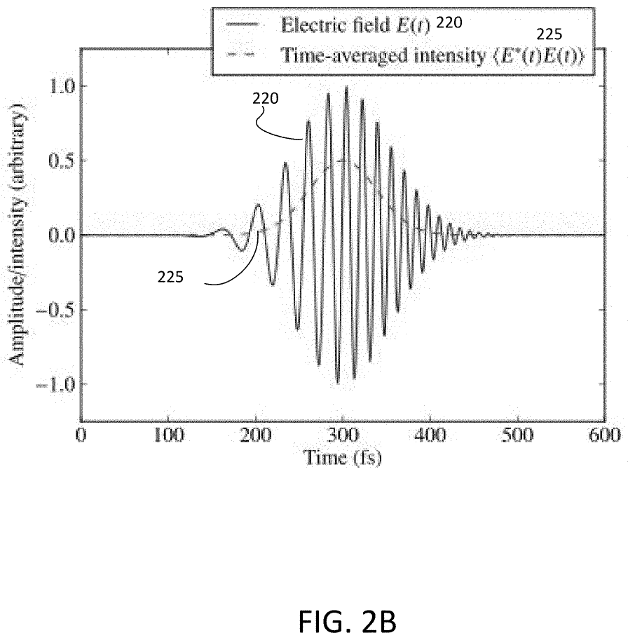

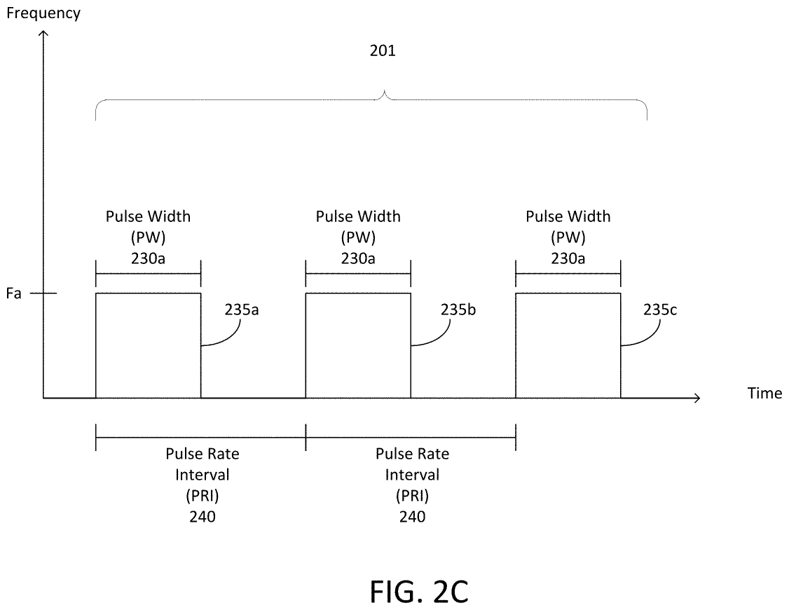

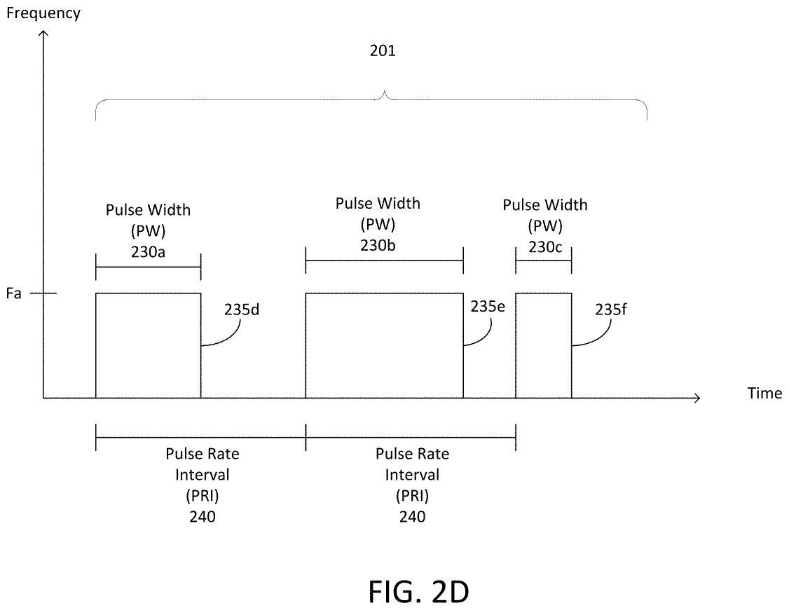

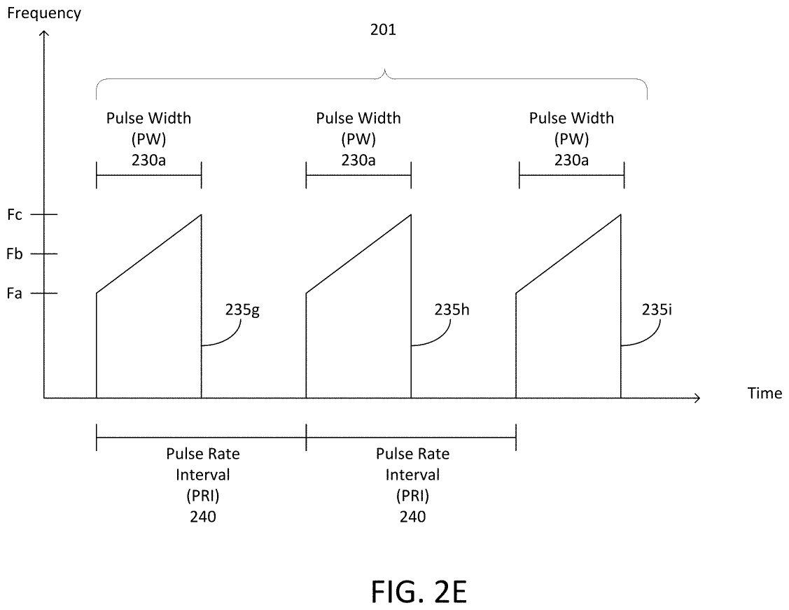

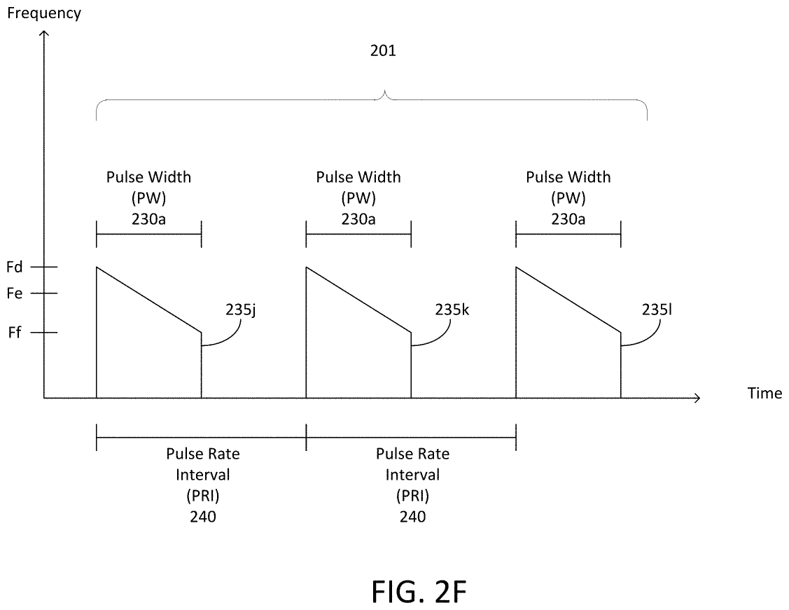

In some embodiments, the light generation module 110 can instruct the visual signaling component 150 to provide a visual signal that include a light wave 200 as depicted in FIG. 2A. The light wave 200 can include or be formed of electromagnetic waves. The electromagnetic waves of the light wave can have respective amplitudes and travel orthogonal to one another as depicted by the amplitude of the electric field 205 versus time and the amplitude of the magnetic field 210 versus time. The light wave 200 can have a wavelength 215. The light wave can also have a frequency. The product of the wavelength 215 and the frequency can be the speed of the light wave. For example, the speed of the light wave can be approximately 299, 792, 458 meters per second in a vacuum.

The light generation module 110 can instruct the visual signaling component 150 to generate light waves having one or more predetermined wavelength or intensity. The wavelength of the light wave can correspond to the visible spectrum, ultraviolet spectrum, infrared spectrum, or some other wavelength of light. For example, the wavelength of the light wave within the visible spectrum range can range from 390 to 700 nanometers (“nm”). Within the visible spectrum, the light generation module 110 can further specify one or more wavelengths corresponding to one or more colors. For example, the light generation module 110 can instruct the visual signaling component 150 to generate visual signals comprising one or more light waves having one or more wavelength corresponding to one or more of ultra-violet (e.g., 10-380 nm); violet (e.g., 380-450 nm), blue (e.g., 450-495 nm), green (e.g., 495-570 nm), yellow (e.g., 570-590 nm), orange (e.g., 590-620 nm), red (e.g., 620-750 nm); or infrared (e.g., 750-1000000 nm). The wavelength can range from 10 nm to 100 micrometers. In some embodiments, the wavelength can be in the range of 380 to 750 nm.

The light generation module 110 can determine to provide visual signals that include light pulses. The light generation module 110 can instruct or otherwise cause the visual signaling component 150 to generate light pulses. A light pulse can refer to a burst of light waves. For example, FIG. 2B illustrates a burst of a light wave. The burst of light wave can refer to a burst of an electric field 250 generated by the light wave. The burst of the electric field 250 of the light wave can be referred to as a light pulse or a flash of light. For example, a light source that is intermittently turned on and off can create bursts, flashes or pulses of light.

FIG. 2C illustrates pulses of light 235a-c in accordance with an embodiment. The light pulses 235a-c can be illustrated via a graph in the frequency spectrum where the y-axis represent frequency of the light wave (e.g., the speed of the light wave divided by the wavelength) and the x-axis represents time. The visual signal can include modulations of light wave between a frequency of Fₐ and frequency different from Fₐ. For example, the NSS 105 can modulate a light wave between a frequency in the visible spectrum, such as Fₐ, and a frequency outside the visible spectrum. The NSS 105 can modulate the light wave between two or more frequencies, between an on state and an off state, or between a high power state and a low power state.

In some cases, the frequency of the light wave used to generate the light pulse can be constant at Fₐ, thereby generating a square wave in the frequency spectrum. In some embodiments, each of the three pulses 235a-c can include light waves having a same frequency Fₐ.

The width of each of the light pulses (e.g., the duration of the burst of the light wave) can correspond to a pulse width 230a. The pulse width 230a can refer to the length or duration of the burst. The pulse width 230a can be measured in units of time or distance. In some embodiments, the pulses 235a-c can include lights waves having different frequencies from one another. In some embodiments, the pulses 235a-c can have different pulse widths 230a from one another, as illustrated in FIG. 2D. For example, a first pulse 235d of FIG. 2D can have a pulse width 230a, while a second pulse 235e has a second pulse width 230b that is greater than the first pulse width 230a. A third pulse 235f can have a third pulse width 230c that is less than the second pulse width 230b. The third pulse width 230c can also be less than the first pulse width 230a. While the pulse widths 230a-c of the pulses 235d-f of the pulse train may vary, the light generation module 110 can maintain a constant pulse rate interval 240 for the pulse train.

The pulses 235a-c can form a pulse train having a pulse rate interval 240. The pulse rate interval 240 can be quantified using units of time. The pulse rate interval 240 can be based on a frequency of the pulses of the pulse train 201. The frequency of the pulses of the pulse train 201 can be referred to as a modulation frequency. For example, the light generation module 110 can provide a pulse train 201 with a predetermined frequency corresponding to gamma activity, such as 40 Hz. To do so, the light generation module 110 can determine the pulse rate interval 240 by taking the multiplicative inverse (or reciprocal) of the frequency (e.g., 1 divided by the predetermined frequency for the pulse train). For example, the light generation module 110 can take the multiplicative inverse of 40 Hz by dividing 1 by 40 Hz to determine the pulse rate interval 240 as 0.025 seconds. The pulse rate interval 240 can remain constant throughout the pulse train. In some embodiments, the pulse rate interval 240 can vary throughout the pulse train or from one pulse train to a subsequent pulse train. In some embodiments, the number of pulses transmitted during a second can be fixed, while the pulse rate interval 240 varies.

In some embodiments, the light generation module 110 can generate a light pulse having a light wave that varies in frequency. For example, the light generation module 110 can generate up-chirp pulses where the frequency of the light wave of the light pulse increases from the beginning of the pulse to the end of the pulse as illustrated in FIG. 2E. For example, the frequency of a light wave at the beginning of pulse 235g can be Fₐ. The frequency of the light wave of the pulse 235g can increase from Fₐ to F_{b} in the middle of the pulse 235g, and then to a maximum of Fc at the end of the pulse 235g. Thus, the frequency of the light wave used to generate the pulse 235g can range from Fₐ to F_{c}. The frequency can increase linearly, exponentially, or based on some other rate or curve.

The light generation module 110 can generate down-chirp pulses, as illustrated in FIG. 2F, where the frequency of the light wave of the light pulse decreases from the beginning of the pulse to the end of the pulse. For example, the frequency of a light wave at the beginning of pulse 235j can be Fₐ. The frequency of the light wave of the pulse 235j can decrease from Fd to Fe in the middle of the pulse 235j, and then to a minimum of Ff at the end of the pulse 235j. Thus, the frequency of the light wave used to generate the pulse 235j can range from F_{d} to F_{f}. The frequency can decrease linearly, exponentially, or based on some other rate or curve.

Visual signaling component 150 can be designed and constructed to generate the light pulses responsive to instructions from the light generation module 110. The instructions can include, for example, parameters of the light pulse such as a frequency or wavelength of the light wave, intensity, duration of the pulse, frequency of the pulse train, pulse rate interval, or duration of the pulse train (e.g., a number of pulses in the pulse train or the length of time to transmit a pulse train having a predetermined frequency). The light pulse can be perceived, observed, or otherwise identified by the brain via ocular means such as eyes. The light pulses can be transmitted to the eye via direct visual field or peripheral visual field.

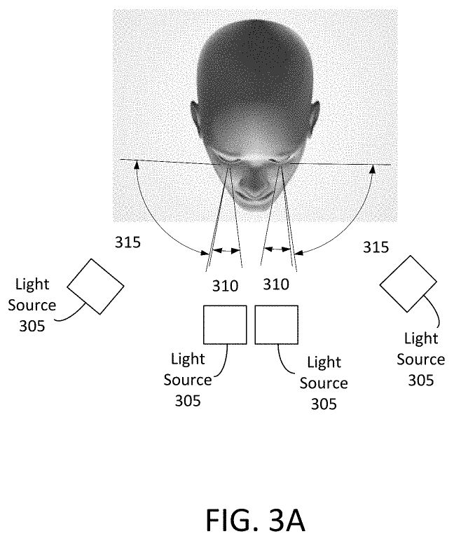

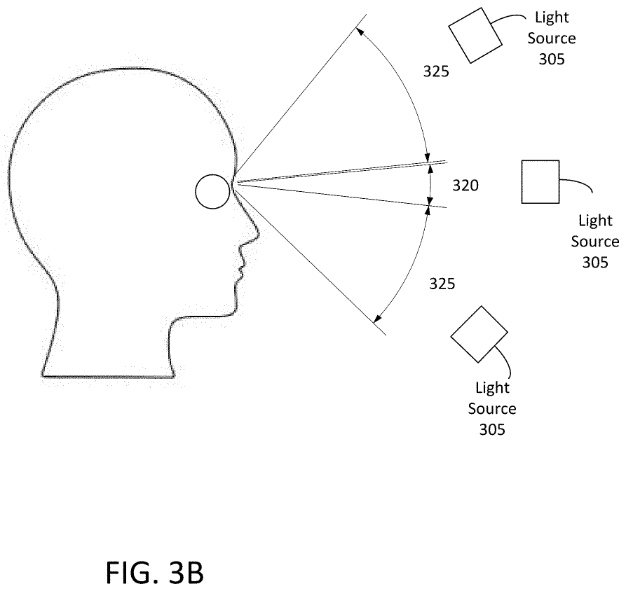

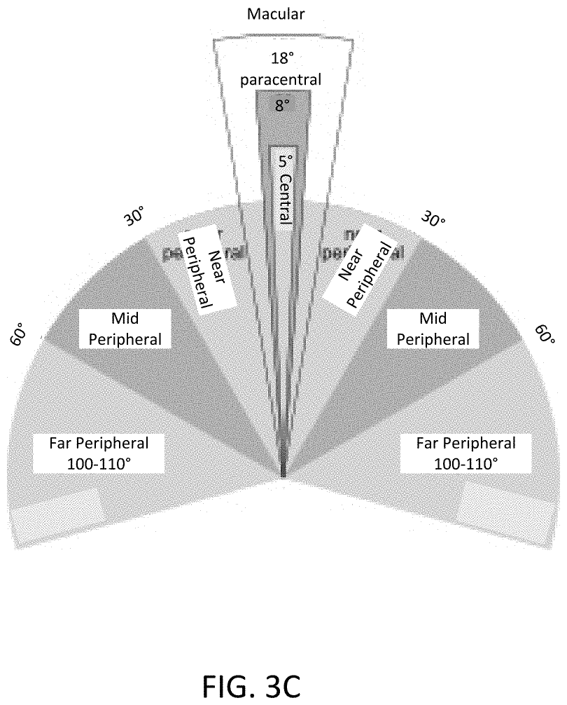

FIG. 3A illustrates a horizontal direct visual field 310 and a horizontal peripheral visual field. FIG. 3B illustrates a vertical direct visual field 320 and a vertical peripheral visual field 325. FIG. 3C illustrates degrees of direct visual fields and peripheral visual fields, including relative distances at which visual signals might be perceived in the different visual fields. The visual signaling component 150 can include a light source 305. The light source 305 can be positioned to transmit light pulses into the direct visual field 310 or 320 of a person's eyes. The NSS 105 can be configured to transmit light pulses into the direct visual field 310 or 320 because this may facilitate brain entrainment as the person may pay more attention to the light pulses. The level of attention can be quantitatively measured directly in the brain, indirectly through the person's eye behavior, or by active feedback (e.g., mouse tracking).

The light source 305 can be positioned to transmit light pulses into a peripheral visual field 315 or 325 of a person's eyes. For example, the NSS 105 can transmit light pulses into the peripheral visual field 315 or 325 as these light pulses may be less distracting to the person who might be performing other tasks, such as reading, walking, driving, etc. Thus, the NSS 105 can provide subtle, on-going visual brain stimulation by transmitting light pulses via the peripheral visual field.

In some embodiments, the light source 305 can be head-worn, while in other embodiments the light source 305 can be held by a subject's hands, placed on a stand, hung from a ceiling, or connected to a chair or otherwise positioned to direct light towards the direct or peripheral visual fields. For example, a chair or externally supported system can include or position the light source 305 to provide the visual input while maintaining a fixed/pre-specified relationship between the subject's visual field and the visual stimulus. The system can provide an immersive experience. For example, the system can include an opaque or partially opaque dome that includes the light source. The dome can be positioned over the subject's head while the subject sits or reclines in chair. The dome can cover portions of the subject's visual field, thereby reducing external distractions and facilitating entrainment of regions of the brain.

The light source 305 can include any type of light source or light emitting device. The light source can include a coherent light source, such as a laser. The light source 305 can include an LED, Organic LED, fluorescent light source, incandescent light, or any other light emitting device. The light source can include a lamp, light bulb, or one or more light emitting diodes of various colors (e.g., white, red, green, blue). In some embodiments, the light source includes a semiconductor light emitting device, such as a light emitting diode of any spectral or wavelength range. In some embodiments, the light source 305 includes a broadband lamp or a broadband light source. In some embodiments, the light source includes a black light. In some embodiments, light source 305 includes a hollow cathode lamp, a fluorescent tube light source, a neon lamp, an argon lamp, a plasma lamp, a xenon flash lamp, a mercury lamp, a metal halide lamp, or a sulfur lamp. In some embodiments, the light source 305 includes a laser, or a laser diode. In some embodiments, light source 305 includes an OLED, PHOLED, QDLED, or any other variation of a light source utilizing an organic material. In some embodiments, light source 305 includes a monochromatic light source. In some embodiments, light source 305 includes a polychromatic light source. In some embodiments, the light source 305 includes a light source emitting light partially in the spectral range of ultraviolet light. In some embodiments, light source 305 includes a device, product or a material emitting light partially in the spectral range of visible light. In some embodiments, light source 305 is a device, product or a material partially emanating or emitting light in the spectral range of the infrared light. In some embodiments, light source 305 includes a device, product or a material emanating or emitting light in the visible spectral range. In some embodiments, light source 305 includes a light guide, an optical fiber or a waveguide through which light is emitted from the light source.

In some embodiments, light source 305 includes one or more mirrors for reflecting or redirecting of light. For example, the mirrors can reflect or redirect light towards the direct visual field 310 or 320, or the peripheral visual field 315 or 325. The light source 305 can include interact with microelectromechanical devices (“MEMS”). The light source 305 can include or interact with a digital light projector (“DLP”). In some embodiments, the light source 305 can include ambient light or sunlight. The ambient light or sunlight can be focused by one or more optical lenses and directed towards the direct visual field or peripheral field. The ambient light or sunlight can be directed by one or more minors towards the directed visual field or peripheral visual field.

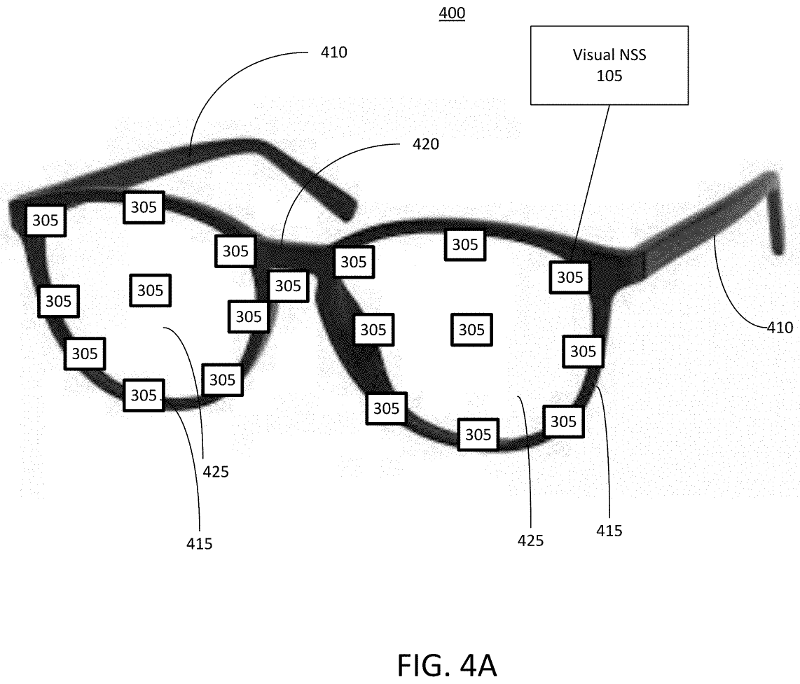

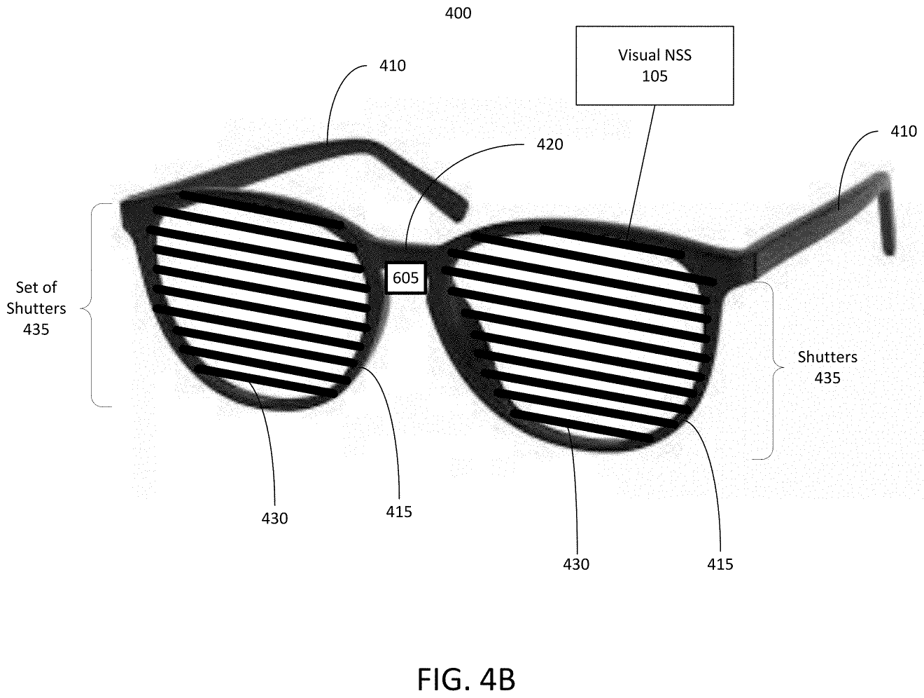

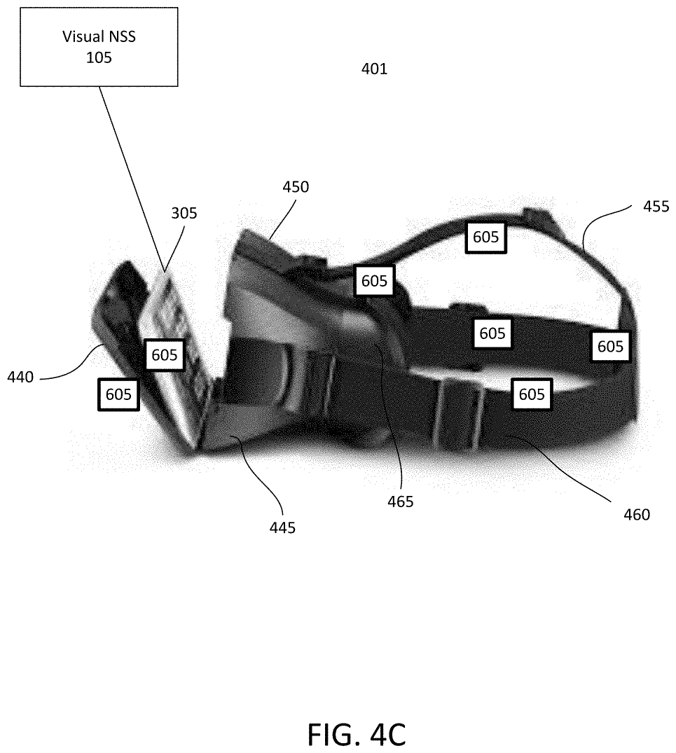

In cases where the light source is ambient light, the ambient light is not positioned but the ambient light can enter the eye via a direct visual field or peripheral visual field. In some embodiments, the light source 305 can be positioned to direct light pulses towards the direct visual field or peripheral field. For example, one or more light sources 305 can be attached, affixed, coupled, mechanically coupled, or otherwise provided with a frame 400 as illustrated in FIG. 4A. In some embodiments, the visual signaling component 150 can include the frame 400. Additional details of the operation of the NSS 105 in conjunction with the frame 400 including one or more light sources 305 are provided below, in the section labelled as “NSS Operating With A Frame”. Thus, the light source can include any type of light source such as an optical light source, mechanical light source, or chemical light source. The light source can include any material or object that is reflective or opaque that can generate, emit, or reflect oscillating patterns of light, such as a fan rotating in front of a light, or bubbles. In some embodiments, the light source can include optical illusions that are invisible, physiological phenomena that are within the eye (e.g., pressing the eyeball), or chemicals applied to the eye.

### Systems and Devices Configured for Neural Stimulation via Visual Stimulation

Referring now to FIG. 4A, the frame 400 can be designed and constructed to be placed or positioned on a person's head. The frame 400 can be configured to be worn by the person. The frame 400 can be designed and constructed to stay in place. The frame 400 can be configured to be worn and stay in place as a person sits, stands, walks, runs, or lays down flat. The light source 305 can be configured on the frame 400 to project light pulses towards the person's eyes during these various positions. In some embodiments, the light source 305 can be configured to project light pulses towards the person's eyes if their eyelids are closed such that the light pulse penetrates the eyelid to be perceived by the retina. The frame 400 can include a bridge 420. The frame 400 can include one or more eye wires 415 coupled to the bridge 420. The bridge 420 can be positioned in between the eye wires 415. The frame 400 can include one or more temples extending from the one or more eye wires 415. In some embodiments, the eye wires 415 can include or hold a lens 425. In some embodiments, the eye wires 415 can include or hold a solid material 425 or cover 425. The lens, solid material, or cover 425 can be transparent, semi-transparent, opaque, or completely block out external light.

One or more light sources 305 can be positioned on or adjacent to the eye wire 415, lens or other solid material 425, or bridge 420. For example, a light source 305 can be positioned in the middle of the eye wire 415 on a solid material 425 in order to transmit light pulses into the direct visual field. In some embodiments, a light source 305 can be positioned at a corner of the eye wire 415, such as a corner of the eye wire 415 coupled to the temple 410, in order to transmit light pulses towards a peripheral field.

The NSS 105 can perform visual brain entrainment via a single eye or both eyes. For example, the NSS 105 can direct light pulses to a single eye or both eyes. The NSS 105 can interface with a visual signaling component 150 that includes a frame 400 and two eye wires 415. However, the visual signaling component 150 may include a single light source 305 configured and positioned to direct light pulses to a first eye. The visual signaling component 150 can further include a light blocking component that keeps out or blocks the light pulses generated from the light source 305 from entering a second eye. The visual signaling component 150 can block or prevent light from entering the second eye during the brain entrainment process.

In some embodiments, the visual signaling component 150 can alternatively transmit or direct light pulses to the first eye and the second eye. For example, the visual signaling component 150 can direct light pulses to the first eye for a first time interval. The visual signaling component 150 can direct light pulses to the second eye for a second time interval. The first time interval and the second time interval can be a same time interval, overlapping time intervals, mutually exclusive time intervals, or subsequent time intervals.

FIG. 4B illustrates a frame 400 comprising a set of shutters 435 that can block at least a portion of light that enters through the eye wire 415. The set of shutters 435 can intermittently block ambient light or sunlight that enters through the eye wire 415. The set of shutters 435 can open to allow light to enter through the eye wire 415, and close to at least partially block light that enters through the eye wire 415. Additional details of the operation of the NSS 105 in conjunction with the frame 400 including one or more shutters 430 are provided below, in the section labelled as “NSS Operating With A Frame”.

The set of shutters 435 can include one or more shutter 430 that is opened and closed by one or more actuator. The shutter 430 can be formed from one or more materials. The shutter 430 can include one or more materials. The shutter 430 can include or be formed from materials that are capable of at least partially blocking or attenuating light.

The frame 400 can include one or more actuators configured to at least partially open or close the set of shutters 435 or an individual shutter 430. The frame 400 can include one or more types of actuators to open and close the shutters 435. For example, the actuator can include a mechanically driven actuator. The actuator can include a magnetically driven actuator. The actuator can include a pneumonic actuator. The actuator can include a hydraulic actuator. The actuator can include a piezoelectric actuator. The actuator can include a micro-electromechanical systems (“MEMS”).

The set of shutters 435 can include one or more shutter 430 that is opened and closed via electrical or chemical techniques. For example, the shutter 430 or set of shutters 435 can be formed from one or more chemicals. The shutter 430 or set of shutters can include one or more chemicals. The shutter 430 or set of shutters 435 can include or be formed from chemicals that are capable of at least partially blocking or attenuating light.

For example, the shutter 430 or set of shutters 435 can include photochromic lenses configured to filter, attenuate or block light. The photochromic lenses can automatically darken when exposed to sunlight. The photochromic lens can include molecules that are configured to darken the lens. The molecules can be activated by light waves, such as ultraviolet radiation or other light wavelengths. Thus, the photochromic molecules can be configured to darken the lens in response to a predetermined wavelength of light.

The shutter 430 or set of shutters 435 can include electrochromic glass or plastic. Electrochromic glass or plastic can change from light to dark (e.g., clear to opaque) in response to an electrical voltage or current. Electrochromic glass or plastic can include metal-oxide coatings that are deposited on the glass or plastic, multiple layers, and lithium ions that travel between two electrodes between a layer to lighten or darken the glass.

The shutter 430 or set of shutters 435 can include micro shutters. Micro shutters can include tiny windows that measure 100 by 200 microns. The micro shutters can be arrayed in the eye frame 415 in a waffle-like grid. The individual micro shutters can be opened or closed by an actuator. The actuator can include a magnetic arm that sweeps past the micro shutter to open or close the micro shutter. An open micro shutter can allow light to enter through the eye frame 415, while a closed micro shutter can block, attenuate, or filter the light.

The NSS 105 can drive the actuator to open and close one or more shutters 430 or the set of shutters 435 at a predetermined frequency such as 40 Hz. By opening and closing the shutter 430 at the predetermined frequency, the shutter 430 can allow flashes of light to pass through the eye wire 415 at the predetermined frequency. Thus, the frame 400 including a set of shutters 435 may not include or use separate light source coupled to the frame 400, such as a light source 305 coupled to frame 400 depicted in FIG. 4A.

In some embodiments, the visual signaling component 150 or light source 305 can refer to or be included in a virtual reality headset 401, as depicted in FIG. 4C. For example, the virtual reality headset 401 can be designed and constructed to receive a light source 305. The light source 305 can include a computing device having a display device, such as a smartphone or mobile telecommunications device. The virtual reality headset 401 can include a cover 440 that opens to receive the light source 305. The cover 440 can close to lock or hold the light source 305 in place. When closed, the cover 440 and case 450 and 445 can form an enclosure for the light source 305. This enclosure can provide an immersive experience that minimize or eliminates unwanted visual distractions. The virtual reality headset can provide an environment to maximize brainwave entrainment. The virtual reality headset can provide an augmented reality experience. In some embodiments, the light source 305 can form an image on another surface such that the image is reflected off the surface and towards a subject's eye (e.g., a heads up display that overlays on the screen a flickering object or an augmented portion of reality). Additional details of the operation of the NSS 105 in conjunction with the virtual reality headset 401 are provided below, in the section labeled as “Systems and Devices Configured for Neural Stimulation Via Visual Stimulation”.

The virtual reality headset 401 includes straps 455 and 460 configured to secure the virtual reality headset 401 to a person's head. The virtual reality headset 401 can be secured via straps 455 and 460 such to minimize movement of the headset 401 worn during physical activity, such as walking or running. The virtual reality headset 401 can include a skull cap formed from 460 or 455.

The feedback sensor 605 can include an electrode, dry electrode, gel electrode, saline soaked electrode, or adhesive-based electrodes.

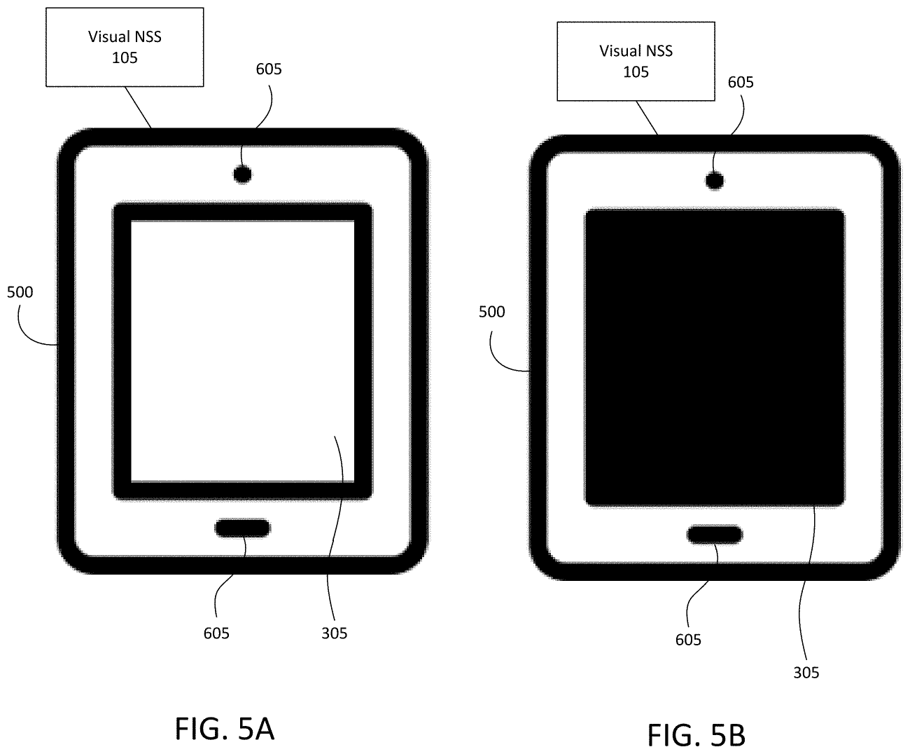

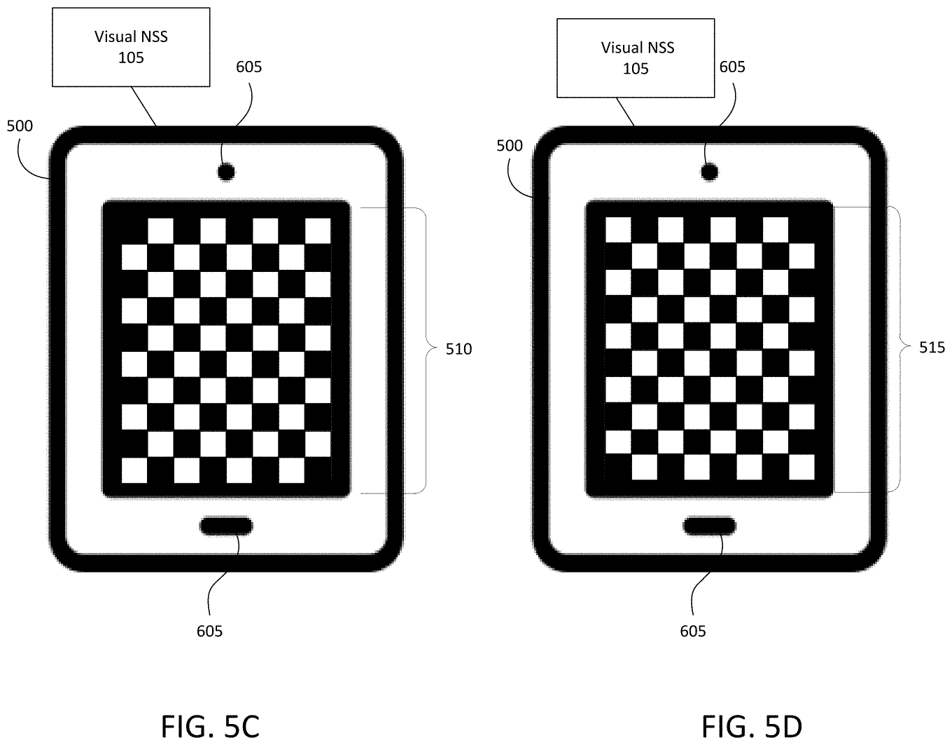

FIGS. 5A-5D illustrate embodiments of the visual signaling component 150 that can include a tablet computing device 500 or other computing device 500 having a display screen 305 as the light source 305. The visual signaling component 150 can transmit light pulses, light flashes, or patterns of light via the display screen 305 or light source 305.

FIG. 5A illustrates a display screen 305 or light source 305 that transmits light. The light source 305 can transmit light comprising a wavelength in the visible spectrum. The NSS 105 can instruct the visual signaling component 150 to transmit light via the light source 305. The NSS 105 can instruct the visual signaling component 150 to transmit flashes of light or light pulses having a predetermined pulse rate interval. For example, FIG. 5B illustrates the light source 305 turned off or disabled such that the light source does not emit light, or emits a minimal or reduced amount of light. The visual signaling component 150 can cause the tablet computing device 500 to enable (e.g., FIG. 5A) and disable (e.g., FIG. 5B) the light source 305 such that flashes of light have a predetermined frequency, such as 40 Hz. The visual signaling component 150 can toggle or switch the light source 305 between two or more states to generate flashes of light or light pulses with the predetermined frequency.

In some embodiments, the light generation module 110 can instruct or cause the visual signaling component 150 to display a pattern of light via display device 305 or light source 305, as depicted in FIGS. 5C and 5D. The light generation module 110 can cause the visual signaling component 150 can flicker, toggle or switch between two or more patterns to generate flashes of light or light pulses. Patterns can include, for example, alternating checkerboard patterns 510 and 515. The pattern can include symbols, characters, or images that can be toggled or adjusted from one state to another state. For example, the color of a character or text relative to a background color can be inverted to cause a switch between a first state 510 and a second state 515. Inverting a foreground color and background color at a predetermined frequency can generate light pulses by way of indicating visual changes that can facilitate adjusting or managing a frequency of neural oscillations. Additional details of the operation of the NSS 105 in conjunction with the tablet 500 are provided below, in the section labeled as “NSS Operating with a Tablet”.

In some embodiments, the light generation module 110 can instruct or cause the visual signaling component 150 to flicker, toggle, or switch between images configured to stimulate specific or predetermined portions of the brain or a specific cortex. The presentation, form, color, motion and other aspects of the light or an image based stimuli can dictate which cortex or cortices are recruited to process the stimuli. The visual signaling component 150 can stimulate discrete portions of the cortex by modulating the presentation of the stimuli to target specific or general regions of interest. The relative position in the field of view, the color of the input, or the motion and speed of the light stimuli can dictate which region of the cortex is stimulated.

For example, the brain can include at least two portions that process predetermined types of visual stimuli: the primary visual cortex on the left side of the brain, and the calcarine fissure on the right side of the brain. Each of these two portions can have one or more multiple sub-portions that process predetermined types of visual stimuli. For example, the calcarine fissure can include a sub-portion referred to as area V5 that can include neurons that respond strongly to motion but may not register stationary objects. Subjects with damage to area V5 may have motion blindness, but otherwise normal vision. In another example, the primary visual cortex can include a sub-portion referred to as area V4 that can include neurons that are specialized for color perception. Subjects with damage to area V4 may have color blindness and only perceive objects in shades of gray. In another example, the primary visual cortex can include a sub-portion referred to as area V1 that includes neurons that respond strongly to contrast edges and helps segment the image into separate objects.

Thus, the light generation module 110 can instruct or cause the visual signaling component 150 to form a type of still image or video, or generate a flicker, or toggle between images that configured to stimulate specific or predetermined portions of the brain or a specific cortex. For example, the light generation module 110 can instruct or cause the visual signaling component 150 to generate images of human faces to stimulate a fusiform face area, which can facilitate brain entrainment for subjects having prosopagnosia or face blindness. The light generation module 110 can instruct or cause the visual signaling component 150 to generate images of faces flickering to target this area of the subject's brain. In another example, the light generation module 110 can instruct the visual signaling component 150 to generate images that include edges or line drawings to stimulate neurons of the primary visual cortex that respond strongly to contrast edges. In some embodiments,

The NSS 105 can include, access, interface with, or otherwise communicate with at least one light adjustment module 115. The light adjustment module 115 can be designed and constructed to measure or verify an environmental variable (e.g., light intensity, timing, incident light, ambient light, eye lid status, etc.) to adjust a parameter associated with the visual signal, such as a frequency, amplitude, wavelength, intensity pattern or other parameter of the visual signal. The light adjustment module 115 can automatically vary a parameter of the visual signal based on profile information or feedback. The light adjustment module 115 can receive the feedback information from the feedback monitor 135. The light adjustment module 115 can receive instructions or information from a side effects management module 130. The light adjustment module 115 can receive profile information from profile manager 125.

The NSS 105 can include, access, interface with, or otherwise communicate with at least one unwanted frequency filtering module 120. The unwanted frequency filtering module 120 can be designed and constructed to block, mitigate, reduce, or otherwise filter out frequencies of visual signals that are undesired to prevent or reduce an amount of such visual signals from being perceived by the brain. The unwanted frequency filtering module 120 can interface, instruct, control, or otherwise communicate with a filtering component 155 to cause the filtering component 155 to block, attenuate, or otherwise reduce the effect of the unwanted frequency on the neural oscillations.

The NSS 105 can include, access, interface with, or otherwise communicate with at least one profile manager 125. The profile manager 125 can be designed or constructed to store, update, retrieve or otherwise manage information associated with one or more subjects associated with the visual brain entrainment. Profile information can include, for example, historical treatment information, historical brain entrainment information, dosing information, parameters of light waves, feedback, physiological information, environmental information, or other data associated with the systems and methods of brain entrainment.

The NSS 105 can include, access, interface with, or otherwise communicate with at least one side effects management module 130. The side effects management module 130 can be designed and constructed to provide information to the light adjustment module 115 or the light generation module 110 to change one or more parameter of the visual signal in order to reduce a side effect. Side effects can include, for example, nausea, migraines, fatigue, seizures, eye strain, or loss of sight.

The side effects management module 130 can automatically instruct a component of the NSS 105 to alter or change a parameter of the visual signal. The side effects management module 130 can be configured with predetermined thresholds to reduce side effects. For example, the side effects management module 130 can be configured with a maximum duration of a pulse train, maximum intensity of light waves, maximum amplitude, maximum duty cycle of a pulse train (e.g., the pulse width multiplied by the frequency of the pulse train), maximum number of treatments for brainwave entrainment in a time period (e.g., 1 hour, 2 hours, 12 hours, or 24 hours).

The side effects management module 130 can cause a change in the parameter of the visual signal in response to feedback information. The side effect management module 130 can receive feedback from the feedback monitor 135. The side effects management module 130 can determine to adjust a parameter of the visual signal based on the feedback. The side effects management module 130 can compare the feedback with a threshold to determine to adjust the parameter of the visual signal.

The side effects management module 130 can be configured with or include a policy engine that applies a policy or a rule to the current visual signal and feedback to determine an adjustment to the visual signal. For example, if feedback indicates that a patient receiving visual signals has a heart rate or pulse rate above a threshold, the side effects management module 130 can turn off the pulse train until the pulse rate stabilizes to a value below the threshold, or below a second threshold that is lower than the threshold.

The NSS 105 can include, access, interface with, or otherwise communicate with at least one feedback monitor 135. The feedback monitor can be designed and constructed to receive feedback information from a feedback component 160. Feedback component 160 can include, for example, a feedback sensor 605 such as a temperature sensor, heart or pulse rate monitor, physiological sensor, ambient light sensor, ambient temperature sensor, sleep status via actigraphy, blood pressure monitor, respiratory rate monitor, brain wave sensor, EEG probe, electrooculography (“EOG”) probes configured to measure the corneo-retinal standing potential that exists between the front and the back of the human eye, accelerometer, gyroscope, motion detector, proximity sensor, camera, microphone, or photo detector.

In some embodiments, a computing device 500 can include the feedback component 160 or feedback sensor 605, as depicted in FIGS. 5C and 5D. For example, the feedback sensor on tablet 500 can include a front-facing camera that can capture images of a person viewing the light source 305.

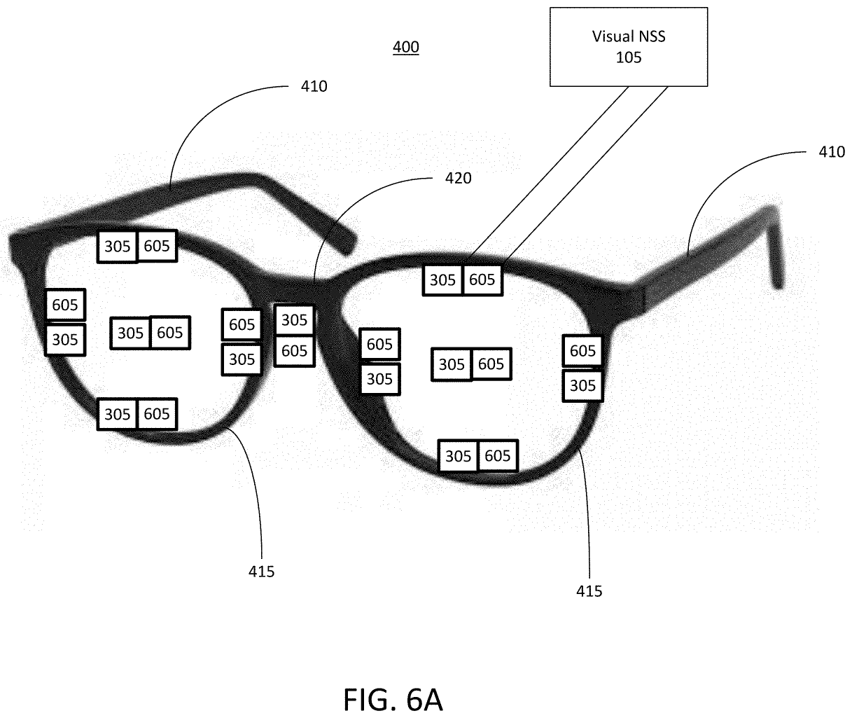

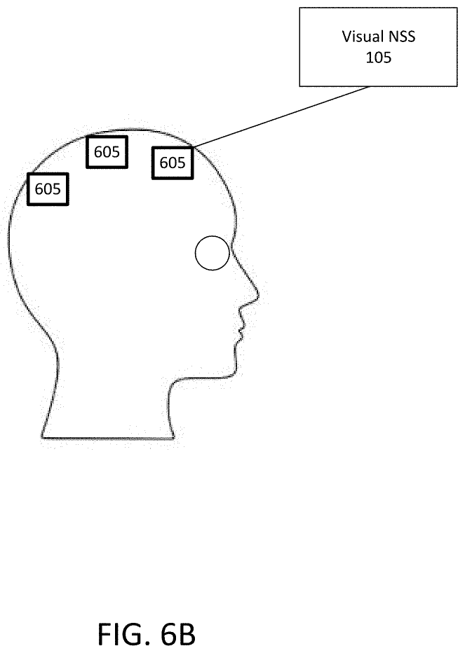

FIG. 6A depicts one or more feedback sensors 605 provided on a frame 400. In some embodiments, a frame 400 can include one or feedback sensors 605 provided on a portion of the frame, such as the bridge 420 or portion of the eye wire 415. The feedback sensor 605 can be provided with or coupled to the light source 305. The feedback sensor 605 can be separate from the light source 305.

The feedback sensor 605 can interact with or communicate with NSS 105. For example, the feedback sensor 605 can provide detected feedback information or data to the NSS 105 (e.g., feedback monitor 135). The feedback sensor 605 can provide data to the NSS 105 in real-time, for example as the feedback sensor 605 detects or senses or information. The feedback sensor 605 can provide the feedback information to the NSS 105 based on a time interval, such as 1 minute, 2 minutes, 5 minutes, 10 minutes, hourly, 2 hours, 4 hours, 12 hours, or 24 hours. The feedback sensor 605 can provide the feedback information to the NSS 105 responsive to a condition or event, such as a feedback measurement exceeding a threshold or falling below a threshold. The feedback sensor 605 can provide feedback information responsive to a change in a feedback parameter. In some embodiments, the NSS 105 can ping, query, or send a request to the feedback sensor 605 for information, and the feedback sensor 605 can provide the feedback information in response to the ping, request, or query.

FIG. 6B illustrates feedback sensors 605 placed or positioned at, on, or near a person's head. Feedback sensors 605 can include, for example, EEG probes that detect brain wave activity.

The feedback monitor 135 can detect, receive, obtain, or otherwise identify feedback information from the one or more feedback sensors 605. The feedback monitor 135 can provide the feedback information to one or more component of the NSS 105 for further processing or storage. For example, the profile manager 125 can update profile data structure 145 stored in data repository 140 with the feedback information. Profile manager 125 can associate the feedback information with an identifier of the patient or person undergoing the visual brain stimulation, as well as a time stamp and date stamp corresponding to receipt or detection of the feedback information.

The feedback monitor 135 can determine a level of attention. The level of attention can refer to the focus provided to the light pulses used for brain stimulation. The feedback monitor 135 can determine the level of attention using various hardware and software techniques. The feedback monitor 135 can assign a score to the level of attention (e.g., 1 to 10 with 1 being low attention and 10 being high attention, or vice versa, 1 to 100 with 1 being low attention and 100 being high attention, or vice versa, 0 to 1 with 0 being low attention and 1 being high attention, or vice versa), categorize the level of attention (e.g., low, medium, high), grade the attention (e.g., A, B, C, D, or F), or otherwise provide an indication of a level of attention.

In some cases, the feedback monitor 135 can track a person's eye movement to identify a level of attention. The feedback monitor 135 can interface with a feedback component 160 that includes an eye-tracker. The feedback monitor 135 (e.g., via feedback component 160) can detect and record eye movement of the person and analyze the recorded eye movement to determine an attention span or level of attention. The feedback monitor 135 can measure eye gaze which can indicate or provide information related to covert attention. For example, the feedback monitor 135 (e.g., via feedback component 160) can be configured with electro-oculography (“EOG”) to measure the skin electric potential around the eye, which can indicate a direction the eye faces relative to the head. In some embodiments, the EOG can include a system or device to stabilize the head so it cannot move in order to determine the direction of the eye relative to the head. In some embodiments, the EOG can include or interface with a head tracker system to determine the position of the heads, and then determine the direction of the eye relative to the head.

In some embodiments, the feedback monitor 135 and feedback component 160 can determine or track the direction of the eye or eye movement using video detection of the pupil or corneal reflection. For example, the feedback component 160 can include one or more camera or video camera. The feedback component 160 can include an infra-red source that sends light pulses towards the eyes. The light can be reflected by the eye. The feedback component 160 can detect the position of the reflection. The feedback component 160 can capture or record the position of the reflection. The feedback component 160 can perform image processing on the reflection to determine or compute the direction of the eye or gaze direction of the eye.

The feedback monitor 135 can compare the eye direction or movement to historical eye direction or movement of the same person, nominal eye movement, or other historical eye movement information to determine a level of attention. For example, if the eye is focused on the light pulses during the pulse train, then the feedback monitor 135 can determine that the level of attention is high. If the feedback monitor 135 determines that the eye moved away from the pulse train for 25% of the pulse train, then the feedback monitor 135 can determine that the level of attention is medium. If the feedback monitor 135 determines that the eye movement occurred for more than 50% of the pulse train or the eye was not focused on the pulse train for greater than 50%, then the feedback monitor 135 can determine that the level of attention is low.

In some embodiments, the system 100 can include a filter (e.g., filtering component 155) to control the spectral range of the light emitted from the light source. In some embodiments, light source includes a light reactive material affecting the light emitted, such as a polarizer, filter, prism or a photochromic material, or electrochromic glass or plastic. The filtering component 155 can receive instructions from the unwanted frequency filtering module 120 to block or attenuate one or more frequencies of light.

The filtering component 155 can include an optical filter that can selectively transmit light in a particular range of wavelengths or colors, while blocking one or more other ranges of wavelengths or colors. The optical filter can modify the magnitude or phase of the incoming light wave for a range of wavelengths. The optical filter can include an absorptive filter, or an interference or dichroic filter. An absorptive filter can take energy of a photon to transform the electromagnetic energy of a light wave into internal energy of the absorber (e.g., thermal energy). The reduction in intensity of a light wave propagating through a medium by absorption of a part of its photons can be referred to as attenuation.

An interference filter or dichroic filter can include an optical filter that reflects one or more spectral bands of light, while transmitting other spectral bands of light. An interference filter or dichroic filter may have a nearly zero coefficient of absorption for one or more wavelengths. Interference filters can be high-pass, low-pass, bandpass, or band-rejection. An interference filter can include one or more thin layers of a dielectric material or metallic material having different refractive indices.

In an illustrative implementation, the NSS 105 can interface with a visual signaling component 150, a filtering component 155, and a feedback component 160. The visual signaling component 150 can include hardware or devices, such as glass frames 400 and one or more light sources 305. The filtering component 155 can include hardware or devices, such as a feedback sensor 605. The filtering component 155 can include hardware, materials or chemicals, such as a polarizing lens, shutters, electrochromic materials or photochromic materials.

### Computing Environment

FIGS. 7A and 7B depict block diagrams of a computing device 700. As shown in FIGS. 7A and 7B, each computing device 700 includes a central processing unit 721, and a main memory unit 722. As shown in FIG. 7A, a computing device 700 can include a storage device 728, an installation device 716, a network interface 718, an I/O controller 723, display devices 724a-724n, a keyboard 726 and a pointing device 727, e.g., a mouse. The storage device 728 can include, without limitation, an operating system, software, and software of a neural stimulation system (“NSS”) 701. The NSS 701 can include or refer to one or more of NSS 105, NSS 905, or NSOS 1605. As shown in FIG. 7B, each computing device 700 can also include additional optional elements, e.g., a memory port 703, a bridge 770, one or more input/output devices 730a-730n (generally referred to using reference numeral 730), and a cache memory 740 in communication with the central processing unit 721.

The central processing unit 721 is any logic circuitry that responds to and processes instructions fetched from the main memory unit 722. In many embodiments, the central processing unit 721 is provided by a microprocessor unit, e.g.: those manufactured by Intel Corporation of Mountain View, California; those manufactured by Motorola Corporation of Schaumburg, Illinois; the ARM processor (from, e.g., ARM Holdings and manufactured by ST, TI, ATMEL, etc.) and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, California; or field programmable gate arrays (“FPGAs”) from Altera in San Jose, Calif., Intel Corporation, Xlinix in San Jose, Calif., or MicroSemi in Aliso Viejo, Calif., etc. The computing device 700 can be based on any of these processors, or any other processor capable of operating as described herein. The central processing unit 721 can utilize instruction level parallelism, thread level parallelism, different levels of cache, and multi-core processors. A multi-core processor can include two or more processing units on a single computing component. Examples of multi-core processors include the AMD PHENOM IIX2, INTEL CORE i5 and INTEL CORE i7.

Main memory unit 722 can include one or more memory chips capable of storing data and allowing any storage location to be directly accessed by the microprocessor 721. Main memory unit 722 can be volatile and faster than storage 728 memory. Main memory units 722 can be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 722 or the storage 728 can be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 722 can be based on any of the above described memory chips, or any other available memory chips capable of operating as described herein. In the embodiment shown in FIG. 7A, the processor 721 communicates with main memory 722 via a system bus 750 (described in more detail below). FIG. 7B depicts an embodiment of a computing device 700 in which the processor communicates directly with main memory 722 via a memory port 703. For example, in FIG. 7B the main memory 722 can be DRDRAM.

FIG. 7B depicts an embodiment in which the main processor 721 communicates directly with cache memory 740 via a secondary bus, sometimes referred to as a backside bus. In other embodiments, the main processor 721 communicates with cache memory 740 using the system bus 750. Cache memory 740 typically has a faster response time than main memory 722 and is typically provided by SRAM, BSRAM, or EDRAM. In the embodiment shown in FIG. 7B, the processor 721 communicates with various I/O devices 730 via a local system bus 750. Various buses can be used to connect the central processing unit 721 to any of the I/O devices 730, including a PCI bus, a PCI-X bus, or a PCI-Express bus, or a NuBus. For embodiments in which the I/O device is a video display 724, the processor 721 can use an Advanced Graphics Port (AGP) to communicate with the display 724 or the I/O controller 723 for the display 724. FIG. 7B depicts an embodiment of a computer 700 in which the main processor 721 communicates directly with I/O device 730b or other processors 721′ via HYPERTRANSPORT, RAPIDIO, or INFINIBAND communications technology. FIG. 7B also depicts an embodiment in which local busses and direct communication are mixed: the processor 721 communicates with I/O device 730a using a local interconnect bus while communicating with I/O device 730b directly.

A wide variety of I/O devices 730a-730n can be present in the computing device 700. Input devices can include keyboards, mice, trackpads, trackballs, touchpads, touch mice, multi-touch touchpads and touch mice, microphones (analog or MEMS), multi-array microphones, drawing tablets, cameras, single-lens reflex camera (SLR), digital SLR (DSLR), CMOS sensors, CCDs, accelerometers, inertial measurement units, infrared optical sensors, pressure sensors, magnetometer sensors, angular rate sensors, depth sensors, proximity sensors, ambient light sensors, gyroscopic sensors, or other sensors. Output devices can include video displays, graphical displays, speakers, headphones, inkjet printers, laser printers, and 3D printers.

Devices 730a-730n can include a combination of multiple input or output devices, including, e.g., Microsoft KINECT, Nintendo Wiimote for the WII, Nintendo WII U GAMEPAD, or Apple IPHONE. Some devices 730a-730n allow gesture recognition inputs through combining some of the inputs and outputs. Some devices 730a-730n provides for facial recognition which can be utilized as an input for different purposes including authentication and other commands. Some devices 730a-730n provides for voice recognition and inputs, including, e.g., Microsoft KINECT, SIRI for IPHONE by Apple, Google Now or Google Voice Search.

Additional devices 730a-730n have both input and output capabilities, including, e.g., haptic feedback devices, touchscreen displays, or multi-touch displays. Touchscreen, multi-touch displays, touchpads, touch mice, or other touch sensing devices can use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices can allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, can have larger surfaces, such as on a table-top or on a wall, and can also interact with other electronic devices. Some I/O devices 730a-730n, display devices 724a-724n or group of devices can be augmented reality devices. The I/O devices can be controlled by an I/O controller 721 as shown in FIG. 7A. The I/O controller 721 can control one or more I/O devices, such as, e.g., a keyboard 126 and a pointing device 727, e.g., a mouse or optical pen. Furthermore, an I/O device can also provide storage and/or an installation medium 116 for the computing device 700. In still other embodiments, the computing device 700 can provide USB connections (not shown) to receive handheld USB storage devices. In further embodiments, an I/O device 730 can be a bridge between the system bus 750 and an external communication bus, e.g., a USB bus, a SCSI bus, a FireWire bus, an Ethernet bus, a Gigabit Ethernet bus, a Fibre Channel bus, or a Thunderbolt bus.

In some embodiments, display devices 724a-724n can be connected to I/O controller 721. Display devices can include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays can use, e.g., stereoscopy, polarization filters, active shutters, or autostereoscopy. Display devices 724a-724n can also be a head-mounted display (HMD). In some embodiments, display devices 724a-724n or the corresponding I/O controllers 723 can be controlled through or have hardware support for OPENGL or DIRECTX API or other graphics libraries.

In some embodiments, the computing device 700 can include or connect to multiple display devices 724a-724n, which each can be of the same or different type and/or form. As such, any of the I/O devices 730a-730n and/or the I/O controller 723 can include any type and/or form of suitable hardware, software, or combination of hardware and software to support, enable or provide for the connection and use of multiple display devices 724a-724n by the computing device 700. For example, the computing device 700 can include any type and/or form of video adapter, video card, driver, and/or library to interface, communicate, connect or otherwise use the display devices 724a-724n. In one embodiment, a video adapter can include multiple connectors to interface to multiple display devices 724a-724n. In other embodiments, the computing device 700 can include multiple video adapters, with each video adapter connected to one or more of the display devices 724a-724n. In some embodiments, any portion of the operating system of the computing device 700 can be configured for using multiple displays 724a-724n. In other embodiments, one or more of the display devices 724a-724n can be provided by one or more other computing devices 700a or 700b connected to the computing device 700, via the network 140. In some embodiments, software can be designed and constructed to use another computer's display device as a second display device 724a for the computing device 700. For example, in one embodiment, an Apple iPad can connect to a computing device 700 and use the display of the device 700 as an additional display screen that can be used as an extended desktop.

Referring again to FIG. 7A, the computing device 700 can comprise a storage device 728 (e.g., one or more hard disk drives or redundant arrays of independent disks) for storing an operating system or other related software, and for storing application software programs such as any program related to the software for the NSS. Examples of storage device 728 include, e.g., hard disk drive (HDD); optical drive including CD drive, DVD drive, or BLU-RAY drive; solid-state drive (SSD); USB flash drive; or any other device suitable for storing data. Some storage devices can include multiple volatile and non-volatile memories, including, e.g., solid state hybrid drives that combine hard disks with solid state cache. Some storage device 728 can be non-volatile, mutable, or read-only. Some storage device 728 can be internal and connect to the computing device 700 via a bus 750. Some storage device 728 can be external and connect to the computing device 700 via a I/O device 730 that provides an external bus. Some storage device 728 can connect to the computing device 700 via the network interface 718 over a network, including, e.g., the Remote Disk for MACBOOK AIR by Apple. Some client devices 700 cannot require a non-volatile storage device 728 and can be thin clients or zero clients 202. Some storage device 728 can also be used as an installation device 716, and can be suitable for installing software and programs. Additionally, the operating system and the software can be run from a bootable medium, for example, a bootable CD, e.g., KNOPPIX, a bootable CD for GNU/Linux that is available as a GNU/Linux distribution from knoppix.net.

Computing device 700 can also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon. com, Inc.

Furthermore, the computing device 700 can include a network interface 718 to interface to the network 140 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 700 communicates with other computing devices 700′ via any type and/or form of gateway or tunneling protocol e.g., Secure Socket Layer (SSL) or Transport Layer Security (TLS), or the Citrix Gateway Protocol manufactured by Citrix Systems, Inc. of Ft. Lauderdale, Fla. The network interface 118 can comprise a built-in network adapter, network interface card, PCMCIA network card, EXPRESSCARD network card, card bus network adapter, wireless network adapter, USB network adapter, modem or any other device suitable for interfacing the computing device 700 to any type of network capable of communication and performing the operations described herein.

A computing device 700 of the sort depicted in FIG. 7A can operate under the control of an operating system, which controls scheduling of tasks and access to system resources. The computing device 700 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 7000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g., Linux Mint distribution (“distro”) or Ubuntu, distributed by Canonical Ltd. Of London, United Kingdom; or Unix or other Unix-like derivative operating systems; and Android, designed by Google, of Mountain View, Calif., among others. Some operating systems, including, e.g., the CHROME OS by Google, can be used on zero clients or thin clients, including, e.g., CHROMEBOOKS.

The computer system 700 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 700 has sufficient processor power and memory capacity to perform the operations described herein. In some embodiments, the computing device 700 can have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 700 is a gaming system. For example, the computer system 700 can comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, or an XBOX 360 device manufactured by the Microsoft Corporation of Redmond, Wash., or an OCULUS RIFT or OCULUS VR device manufactured BY OCULUS VR, LLC of Menlo Park, Calif.

In some embodiments, the computing device 700 is a digital audio player such as the Apple IPOD, IPOD Touch, and IPOD NANO lines of devices, manufactured by Apple Computer of Cupertino, Calif. Some digital audio players can have other functionality, including, e.g., a gaming system or any functionality made available by an application from a digital application distribution platform. For example, the IPOD Touch can access the Apple App Store. In some embodiments, the computing device 700 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 700 is a tablet e.g., the IPAD line of devices by Apple; GALAXY TAB family of devices by Samsung; or KINDLE FIRE, by Amazon.com, Inc. of Seattle, Wash. In other embodiments, the computing device 700 is an eBook reader, e.g., the KINDLE family of devices by Amazon.com, or NOOK family of devices by Barnes & Noble, Inc. of New York City, N.Y.

In some embodiments, the communications device 700 includes a combination of devices, e.g., a smartphone combined with a digital audio player or portable media player. For example, one of these embodiments is a smartphone, e.g., the IPHONE family of smartphones manufactured by Apple, Inc.; a Samsung GALAXY family of smartphones manufactured by Samsung, Inc.; or a Motorola DROID family of smartphones. In yet another embodiment, the communications device 700 is a laptop or desktop computer equipped with a web browser and a microphone and speaker system, e.g., a telephony headset. In these embodiments, the communications devices 700 are web-enabled and can receive and initiate phone calls. In some embodiments, a laptop or desktop computer is also equipped with a webcam or other video capture device that enables video chat and video call.

In some embodiments, the status of one or more machines 700 in the network are monitored, generally as part of network management. In one of these embodiments, the status of a machine can include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information can be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### A Method for Neural Stimulation

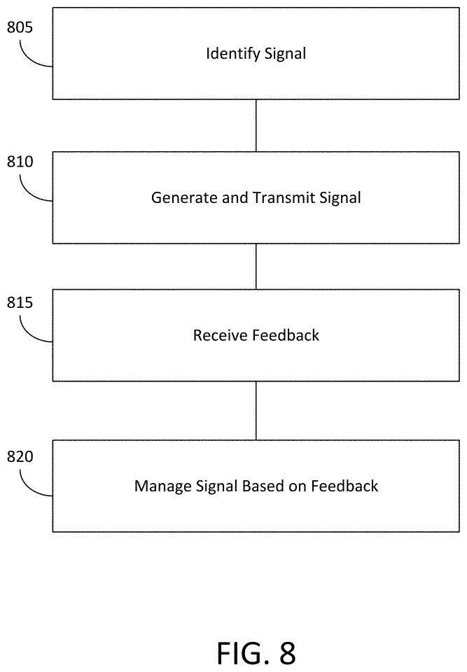

In FIG. 8 is a flow diagram of a method of performing visual brain entrainment in accordance with an embodiment. The method 800 can be performed by one or more system, component, module or element depicted in FIGS. 1-7B, including, for example, a neural stimulation system (NSS). In brief overview, the NSS can identify a visual signal to provide at block 805. At block 810, the NSS can generate and transmit the identified visual signal. At 815 the NSS can receive or determine feedback associated with neural activity, physiological activity, environmental parameters, or device parameters. At 820 the NSS can manage, control, or adjust the visual signal based on the feedback.

NSS Operating with a Frame

The NSS 105 can operate in conjunction with the frame 400 including a light source 305 as depicted in FIG. 4A. The NSS 105 can operate in conjunction with the frame 400 including a light source 30 and a feedback sensor 605 as depicted in FIG. 6A. The NSS 105 can operate in conjunction with the frame 400 including at least one shutter 430 as depicted in FIG. 4B. The NSS 105 can operate in conjunction with the frame 400 including at least one shutter 430 and a feedback sensor 605.

In operation, a user of the frame 400 can wear the frame 400 on their head such that eye wires 415 encircle or substantially encircle their eyes. In some cases, the user can provide an indication to the NSS 105 that the glass frames 400 have been worn and that the user is ready to undergo brainwave entrainment. The indication can include an instruction, command, selection, input, or other indication via an input/output interface, such as a keyboard 726, pointing device 727, or other I/O devices 730a-n. The indication can be a motion-based indication, visual indication, or voice-based indication. For example, the user can provide a voice command that indicates that the user is ready to undergo brainwave entrainment.

In some cases, the feedback sensor 605 can determine that the user is ready to undergo brainwave entrainment. The feedback sensor 605 can detect that the glass frames 400 have been placed on a user's head. The NSS 105 can receive motion data, acceleration data, gyroscope data, temperature data, or capacitive touch data to determine that the frames 400 have been placed on the user's head. The received data, such as motion data, can indicate that the frames 400 were picked up and placed on the user's head. The temperature data can measure the temperature of or proximate to the frames 400, which can indicate that the frames are on the user's head. In some cases, the feedback sensor 605 can perform eye tracking to determine a level of attention a user is paying to the light source 305 or feedback sensor 605. The NSS 105 can detect that the user is ready responsive to determining that the user is paying a high level of attention to the light source 305 or feedback sensor 605. For example, staring at, gazing or looking in the direction of the light source 305 or feedback sensor 605 can provide an indication that the user is ready to undergo brainwave entrainment.

Thus, the NSS 105 can detect or determine that the frames 400 have been worn and that the user is in a ready state, or the NSS 105 can receive an indication or confirmation from the user that the user has worn the frames 400 and the user is ready to undergo brainwave entrainment. Upon determining that the user is ready, the NSS 105 can initialize the brainwave entrainment process. In some embodiments, the NSS 105 can access a profile data structure 145. For example, a profile manager 125 can query the profile data structure 145 to determine one or more parameter for the external visual stimulation used for the brain entrainment process. Parameters can include, for example, a type of visual stimulation, an intensity of the visual stimulation, frequency of the visual stimulation, duration of the visual stimulation, or wavelength of the visual stimulation. The profile manager 125 can query the profile data structure 145 to obtain historical brain entrainment information, such as prior visual stimulation sessions. The profile manager 125 can perform a lookup in the profile data structure 145. The profile manager 125 can perform a look-up with a username, user identifier, location information, fingerprint, biometric identifier, retina scan, voice recognition and authentication, or other identifying technique.

The NSS 105 can determine a type of external visual stimulation based on the hardware 400. The NSS 105 can determine the type of external visual stimulation based on the type of light source 305 available. For example, if the light source 305 includes a monochromatic LED that generates light waves in the red spectrum, the NSS 105 can determine that the type of visual stimulation includes pulses of light transmitted by the light source. However, if the frames 400 do not include an active light source 305, but, instead, include one or more shutters 430, the NSS 105 can determine that the light source is sunlight or ambient light that is to be modulated as it enters the user's eye via a plane formed by the eye wire 415.

In some embodiments, the NSS 105 can determine the type of external visual stimulation based on historical brainwave entrainment sessions. For example, the profile data structure 145 can be pre-configured with information about the type of visual signaling component 150.

The NSS 105 can determine, via the profile manager 125, a modulation frequency for the pulse train or the ambient light. For example, NSS 105 can determine, from the profile data structure 145, that the modulation frequency for the external visual stimulation should be set to 40 Hz. Depending on the type of visual stimulation, the profile data structure 145 can further indicate a pulse length, intensity, wavelength of the light wave forming the light pulse, or duration of the pulse train.

In some cases, the NSS 105 can determine or adjust one or more parameter of the external visual stimulation. For example, the NSS 105 (e.g., via feedback component 160 or feedback sensor 605) can determine a level or amount of ambient light. The NSS 105 (e.g., via light adjustment module 115 or side effects management module 130) can establish, initialize, set, or adjust the intensity or wavelength of the light pulse. For example, the NSS 105 can determine that there is a low level of ambient light. Due to the low level of ambient light, the user's pupils may be dilated. The NSS 105 can determine, based on detecting a low level of ambient light, that the user's pupils are likely dilated. In response to determining that the user's pupils are likely dilated, the NSS 105 can set a low level of intensity for the pulse train. The NSS 105 can further use a light wave having a longer wavelength (e.g., red), which may reduce strain on the eyes.

In some embodiments, the NSS 105 can monitor (e.g., via feedback monitor 135 and feedback component 160) the level of ambient light throughout the brainwave entrainment process to automatically and periodically adjust the intensity or color of light pulses. For example, if the user began the brainwave entrainment process when there was a high level of ambient light, the NSS 105 can initially set a higher intensity level for the light pulses and use a color that includes light waves having lower wavelengths (e.g., blue). However, in some embodiments in which the ambient light level decreases throughout the brainwave entrainment process, the NSS 105 can automatically detect the decrease in ambient light and, in response to the detection, adjust or lower the intensity while increasing the wavelength of the light wave. The NSS 105 can adjust the light pulses to provide a high contrast ratio to facilitate brainwave entrainment.

In some embodiments, the NSS 105 (e.g., via feedback monitor 135 and feedback component 160) can monitor or measure physiological conditions to set or adjust a parameter of the light wave. For example, the NSS 105 can monitor or measure a level of pupil dilation to adjust or set a parameter of the light wave. In some embodiments, the NSS 105 can monitor or measure heart rate, pulse rate, blood pressure, body temperature, perspiration, or brain activity to set or adjust a parameter of the light wave.

In some embodiments, the NSS 105 can be preconfigured to initially transmit light pulses having a lowest setting for light wave intensity (e.g., low amplitude of the light wave or high wavelength of the light wave) and gradually increase the intensity (e.g., increase the amplitude of the light wave or decrease the wavelength of the light wave) while monitoring feedback until an optimal light intensity is reached. An optimal light intensity can refer to a highest intensity without adverse physiological side effects, such as blindness, seizures, heart attack, migraines, or other discomfort. The NSS 105 (e.g., via side effects management module 130) can monitor the physiological symptoms to identify the adverse side effects of the external visual stimulation, and adjust (e.g., via light adjustment module 115) the external visual stimulation accordingly to reduce or eliminate the adverse side effects.

In some embodiments, the NSS 105 (e.g., via light adjustment module 115) can adjust a parameter of the light wave or light pulse based on a level of attention. For example, during the brainwave entrainment process, the user may get bored, lose focus, fall asleep, or otherwise not pay attention to the light pulses. Not paying attention to the light pulses may reduce the efficacy of the brainwave entrainment process, resulting in neurons oscillating at a frequency different from the desired modulation frequency of the light pulses.

NSS 105 can detect the level of attention the user is paying to the light pulses using the feedback monitor 135 and one or more feedback component 160. The NSS 105 can perform eye tracking to determine the level of attention the user is providing to the light pulses based on the gaze direction of the retina or pupil. The NSS 105 can measure eye movement to determine the level of attention the user is paying to the light pulses. The NSS 105 can provide a survey or prompt asking for user feedback that indicates the level of attention the user is paying to the light pulses. Responsive to determining that the user is not paying a satisfactory amount of attention to the light pulses (e.g., a level of eye movement that is greater than a threshold or a gaze direction that is outside the direct visual field of the light source 305), the light adjustment module 115 can change a parameter of the light source to gain the user's attention. For example, the light adjustment module 115 can increase the intensity of the light pulse, adjust the color of the light pulse, or change the duration of the light pulse. The light adjustment module 115 can randomly vary one or more parameters of the light pulse. The light adjustment module 115 can initiate an attention seeking light sequence configured to regain the user's attention. For example, the light sequence can include a change in color or intensity of the light pulses in a predetermined, random, or pseudo-random pattern. The attention seeking light sequence can enable or disable different light sources if the visual signaling component 150 includes multiple light sources. Thus, the light adjustment module 115 can interact with the feedback monitor 135 to determine a level of attention the user is providing to the light pulses, and adjust the light pulses to regain the user's attention if the level of attention falls below a threshold.

In some embodiments, the light adjustment module 115 can change or adjust one or more parameter of the light pulse or light wave at predetermined time intervals (e.g., every 5 minutes, 10 minutes, 15 minutes, or 20 minutes) to regain or maintain the user's attention level.

In some embodiments, the NSS 105 (e.g., via unwanted frequency filtering module 120) can filter, block, attenuate, or remove unwanted visual external stimulation. Unwanted visual external stimulation can include, for example, unwanted modulation frequencies, unwanted intensities, or unwanted wavelengths of light waves. The NSS 105 can deem a modulation frequency to be unwanted if the modulation frequency of a pulse train is different or substantially different (e.g., 1%, 2%, 5%, 10%, 15%, 20%, 25%, or more than 25%) from a desired frequency.

For example, the desired modulation frequency for brainwave entrainment can be 40 Hz. However, a modulation frequency of 20 Hz or 80 Hz can hinder brainwave entrainment. Thus, the NSS 105 can filter out the light pulses or light waves corresponding to the 20 Hz or 80 Hz modulation frequency.

In some embodiments, the NSS 105 can detect, via feedback component 160, that there are light pulses from an ambient light source that corresponds to an unwanted modulation frequency of 20 Hz. The NSS 105 can further determine the wavelength of the light waves of the light pulses corresponding to the unwanted modulation frequency. The NSS 105 can instruct the filtering component 155 to filter out the wavelength corresponding to the unwanted modulation frequency. For example, the wavelength corresponding to the unwanted modulation frequency can correspond to the color blue. The filtering component 155 can include an optical filter that can selectively transmit light in a particular range of wavelengths or colors, while blocking one or more other ranges of wavelengths or colors. The optical filter can modify the magnitude or phase of the incoming light wave for a range of wavelengths. For example, the optical filter can be configured to block, reflect or attenuate the blue light wave corresponding to the unwanted modulation frequency. The light adjustment module 115 can change the wavelength of the light wave generated by the light generation module 110 and light source 305 such that the desired modulation frequency is not blocked or attenuated by the unwanted frequency filtering module 120.

NSS Operating with a Virtual Reality Headset

The NSS 105 can operate in conjunction with the virtual reality headset 401 including a light source 305 as depicted in FIG. 4C. The NSS 105 can operate in conjunction with the virtual reality headset 401 including a light source 305 and a feedback sensor 605 as depicted in FIG. 4C. In some embodiments, the NSS 105 can determine that the visual signaling component 150 hardware includes a virtual reality headset 401. Responsive to determining that the visual signaling component 150 includes a virtual reality headset 401, the NSS 105 can determine that the light source 305 includes a display screen of a smartphone or other mobile computing device.

The virtual reality headset 401 can provide an immersive, non-disruptive visual stimulation experience. The virtual reality headset 401 can provide an augmented reality experience. The feedback sensors 605 can capture pictures or video of the physical, real world to provide the augmented reality experience. The unwanted frequency filtering module 120 can filter out unwanted modulation frequencies prior to projecting, displaying or providing the augmented reality images via the display screen 305.

In operation, a user of the frame 401 can wear the frame 401 on their head such that the virtual reality headset eye sockets 465 cover the user's eyes. The virtual reality headset eye sockets 465 can encircle or substantially encircle their eyes. The user can secure the virtual reality headset 401 to the user's headset using one or more straps 455 or 460, a skull cap, or other fastening mechanism. In some cases, the user can provide an indication to the NSS 105 that the virtual reality headset 401 has been placed and secured to the user's head and that the user is ready to undergo brainwave entrainment. The indication can include an instruction, command, selection, input, or other indication via an input/output interface, such as a keyboard 726, pointing device 727, or other I/O devices 730a-n. The indication can be a motion-based indication, visual indication, or voice-based indication. For example, the user can provide a voice command that indicates that the user is ready to undergo brainwave entrainment.

In some cases, the feedback sensor 605 can determine that the user is ready to undergo brainwave entrainment. The feedback sensor 605 can detect that the virtual reality headset 401 has been placed on a user's head. The NSS 105 can receive motion data, acceleration data, gyroscope data, temperature data, or capacitive touch data to determine that the virtual reality headset 401 has been placed on the user's head. The received data, such as motion data, can indicate that the virtual reality headset 401 was picked up and placed on the user's head. The temperature data can measure the temperature of or proximate to the virtual reality headset 401, which can indicate that the virtual reality headset 401 is on the user's head. In some cases, the feedback sensor 605 can perform eye tracking to determine a level of attention a user is paying to the light source 305 or feedback sensor 605. The NSS 105 can detect that the user is ready responsive to determining that the user is paying a high level of attention to the light source 305 or feedback sensor 605. For example, staring at, gazing or looking in the direction of the light source 305 or feedback sensor 605 can provide an indication that the user is ready to undergo brainwave entrainment.

In some embodiments, a sensor 605 on the straps 455, straps 460 or eye socket 605 can detect that the virtual reality headset 401 is secured, placed, or positioned on the user's head. The sensor 605 can be a touch sensor that senses or detects the touch of the user's head.

Thus, the NSS 105 can detect or determine that the virtual reality headset 401 has been worn and that the user is in a ready state, or the NSS 105 can receive an indication or confirmation from the user that the user has worn the virtual reality headset 401 and the user is ready to undergo brainwave entrainment. Upon determining that the user is ready, the NSS 105 can initialize the brainwave entrainment process. In some embodiments, the NSS 105 can access a profile data structure 145. For example, a profile manager 125 can query the profile data structure 145 to determine one or more parameter for the external visual stimulation used for the brain entrainment process. Parameters can include, for example, a type of visual stimulation, an intensity of the visual stimulation, frequency of the visual stimulation, duration of the visual stimulation, or wavelength of the visual stimulation. The profile manager 125 can query the profile data structure 145 to obtain historical brain entrainment information, such as prior visual stimulation sessions. The profile manager 125 can perform a lookup in the profile data structure 145. The profile manager 125 can perform a look-up with a username, user identifier, location information, fingerprint, biometric identifier, retina scan, voice recognition and authentication, or other identifying technique.

The NSS 105 can determine a type of external visual stimulation based on the hardware 401. The NSS 105 can determine the type of external visual stimulation based on the type of light source 305 available. For example, if the light source 305 includes a smartphone or display device, the visual stimulation can include turning on and off the display screen of the display device. The visual stimulation can include displaying a pattern on the display device 305, such as a checkered pattern, that can alternate in accordance with the desired frequency modulation. The visual stimulation can include light pulses generated by a light source 305 such as an LED that is placed within the virtual reality headset 401 enclosure.

In cases where the virtual reality headset 401 provides an augmented reality experience, the visual stimulation can include overlaying content on the display device and modulating the overlaid content at the desired modulation frequency. For example, the virtual reality headset 401 can include a camera 605 that captures the real, physical world. While displaying the captured image of the real, physical world, the NSS 105 can also display content that is modulated at the desired modulation frequency. The NSS 105 can overlay the content modulated at the desired modulation frequency. The NSS 105 can otherwise modify, manipulate, modulation, or adjust a portion of the display screen or a portion of the augmented reality to generate or provide the desired modulation frequency.

For example, the NSS 105 can modulate one or more pixels based on the desired modulation frequency. The NSS 105 can turn pixels on and off based on the modulation frequency. The NSS 105 can turn of pixels on any portion of the display device. The NSS 105 can turn on and off pixels in a pattern. The NSS 105 can turn on and off pixels in the direct visual field or peripheral visual field. The NSS 105 can track or detect a gaze direction of the eye and turn on and off pixels in the gaze direction so the light pulses (or modulation) are in the direct vision field. Thus, modulating the overlaid content or otherwise manipulated the augmented reality display or other image provided via a display device in the virtual reality headset 401 can generate light pulses or light flashes having a modulation frequency configured to facilitate brainwave entrainment.

The NSS 105 can determine, via the profile manager 125, a modulation frequency for the pulse train or the ambient light. For example, NSS 105 can determine, from the profile data structure 145, that the modulation frequency for the external visual stimulation should be set to 40 Hz. Depending on the type of visual stimulation, the profile data structure 145 can further indicate a number of pixels to modulate, intensity of pixels to modulate, pulse length, intensity, wavelength of the light wave forming the light pulse, or duration of the pulse train.

In some cases, the NSS 105 can determine or adjust one or more parameter of the external visual stimulation. For example, the NSS 105 (e.g., via feedback component 160 or feedback sensor 605) can determine a level or amount of light in captured image used to provide the augmented reality experience. The NSS 105 (e.g., via light adjustment module 115 or side effects management module 130) can establish, initialize, set, or adjust the intensity or wavelength of the light pulse based on the light level in the image data corresponding to the augmented reality experience. For example, the NSS 105 can determine that there is a low level of light in the augmented reality display because it may be dark outside. Due to the low level of light in the augmented reality display, the user's pupils may be dilated. The NSS 105 can determine, based on detecting a low level of light, that the user's pupils are likely dilated. In response to determining that the user's pupils are likely dilated, the NSS 105 can set a low level of intensity for the light pulses or light source providing the modulation frequency. The NSS 105 can further use a light wave having a longer wavelength (e.g., red), which may reduce strain on the eyes.

In some embodiments, the NSS 105 can monitor (e.g., via feedback monitor 135 and feedback component 160) the level of light throughout the brainwave entrainment process to automatically and periodically adjust the intensity or color of light pulses. For example, if the user began the brainwave entrainment process when there was a high level of ambient light, the NSS 105 can initially set a higher intensity level for the light pulses and use a color that includes light waves having lower wavelengths (e.g., blue). However, as the light level decreases throughout the brainwave entrainment process, the NSS 105 can automatically detect the decrease in light and, in response to the detection, adjust or lower the intensity while increasing the wavelength of the light wave. The NSS 105 can adjust the light pulses to provide a high contrast ratio to facilitate brainwave entrainment.

In some embodiments, the NSS 105 (e.g., via feedback monitor 135 and feedback component 160) can monitor or measure physiological conditions to set or adjust a parameter of the light pulses while the user is wearing the virtual reality headset 401. For example, the NSS 105 can monitor or measure a level of pupil dilation to adjust or set a parameter of the light wave. In some embodiments, the NSS 105 can monitor or measure, via one or more feedback sensor of the virtual reality headset 401 or other feedback sensor, a heart rate, pulse rate, blood pressure, body temperature, perspiration, or brain activity to set or adjust a parameter of the light wave.

In some embodiments, the NSS 105 can be preconfigured to initially transmit, via display device 305, light pulses having a lowest setting for light wave intensity (e.g., low amplitude of the light wave or high wavelength of the light wave) and gradually increase the intensity (e.g., increase the amplitude of the light wave or decrease the wavelength of the light wave) while monitoring feedback until an optimal light intensity is reached. An optimal light intensity can refer to a highest intensity without adverse physiological side effects, such as blindness, seizures, heart attack, migraines, or other discomfort. The NSS 105 (e.g., via side effects management module 130) can monitor the physiological symptoms to identify the adverse side effects of the external visual stimulation, and adjust (e.g., via light adjustment module 115) the external visual stimulation accordingly to reduce or eliminate the adverse side effects.

In some embodiments, the NSS 105 (e.g., via light adjustment module 115) can adjust a parameter of the light wave or light pulse based on a level of attention. For example, during the brainwave entrainment process, the user may get bored, lose focus, fall asleep, or otherwise not pay attention to the light pulses generated via the display screen 305 of the virtual reality headset 401. Not paying attention to the light pulses may reduce the efficacy of the brainwave entrainment process, resulting in neurons oscillating at a frequency different from the desired modulation frequency of the light pulses.

NSS 105 can detect the level of attention the user is paying or providing to the light pulses using the feedback monitor 135 and one or more feedback component 160 (e.g., including feedback sensors 605). The NSS 105 can perform eye tracking to determine the level of attention the user is providing to the light pulses based on the gaze direction of the retina or pupil. The NSS 105 can measure eye movement to determine the level of attention the user is paying to the light pulses. The NSS 105 can provide a survey or prompt asking for user feedback that indicates the level of attention the user is paying to the light pulses. Responsive to determining that the user is not paying a satisfactory amount of attention to the light pulses (e.g., a level of eye movement that is greater than a threshold or a gaze direction that is outside the direct visual field of the light source 305), the light adjustment module 115 can change a parameter of the light source 305 or display device 305 to gain the user's attention. For example, the light adjustment module 115 can increase the intensity of the light pulse, adjust the color of the light pulse, or change the duration of the light pulse. The light adjustment module 115 can randomly vary one or more parameters of the light pulse. The light adjustment module 115 can initiate an attention seeking light sequence configured to regain the user's attention. For example, the light sequence can include a change in color or intensity of the light pulses in a predetermined, random, or pseudo-random pattern. The attention seeking light sequence can enable or disable different light sources if the visual signaling component 150 includes multiple light sources. Thus, the light adjustment module 115 can interact with the feedback monitor 135 to determine a level of attention the user is providing to the light pulses, and adjust the light pulses to regain the user's attention if the level of attention falls below a threshold.

In some embodiments, the light adjustment module 115 can change or adjust one or more parameter of the light pulse or light wave at predetermined time intervals (e.g., every 5 minutes, 10 minutes, 15 minutes, or 20 minutes) to regain or maintain the user's attention level.

In some embodiments, the NSS 105 (e.g., via unwanted frequency filtering module 120) can filter, block, attenuate, or remove unwanted visual external stimulation. Unwanted visual external stimulation can include, for example, unwanted modulation frequencies, unwanted intensities, or unwanted wavelengths of light waves. The NSS 105 can deem a modulation frequency to be unwanted if the modulation frequency of a pulse train is different or substantially different (e.g., 1%, 2%, 5%, 10%, 15%, 20%, 25%, or more than 25%) from a desired frequency.

For example, the desired modulation frequency for brainwave entrainment can be 40 Hz. However, a modulation frequency of 20 Hz or 80 Hz can hinder brainwave entrainment. Thus, the NSS 105 can filter out the light pulses or light waves corresponding to the 20 Hz or 80 Hz modulation frequency. For example, the virtual reality headset 401 can detect unwanted modulation frequencies in the physical, real world and eliminate, attenuate, filter out or otherwise remove the unwanted frequencies providing to generating the or providing the augmented reality experience. The NSS 105 can include an optical filter configured to perform digital signal processing or digital image processing to detect the unwanted modulation frequency in the real world captured by the feedback sensor 605. The NSS 105 can detect other content, image or motion having an unwanted parameter (e.g., color, brightness, contrast ratio, modulation frequency), and eliminate same from the augmented reality experience projected to the user via the display screen 305. The NSS 105 can apply a color filter to adjust the color or remove a color of the augmented reality display. The NSS 105 can adjust, modify, or manipulate the brightness, contrast ratio, sharpness, tint, hue, or other parameter of the image or video displayed via the display device 305.

In some embodiments, the NSS 105 can detect, via feedback component 160, that there is captured image or video content from the real, physical world that corresponds to an unwanted modulation frequency of 20 Hz. The NSS 105 can further determine the wavelength of the light waves of the light pulses corresponding to the unwanted modulation frequency. The NSS 105 can instruct the filtering component 155 to filter out the wavelength corresponding to the unwanted modulation frequency. For example, the wavelength corresponding to the unwanted modulation frequency can correspond to the color blue. The filtering component 155 can include a digital optical filter that can digitally remove content or light in a particular range of wavelengths or colors, while allowing one or more other ranges of wavelengths or colors. The digital optical filter can modify the magnitude or phase of the image for a range of wavelengths. For example, the digital optical filter can be configured to attenuate, erase, replace or otherwise alter the blue light wave corresponding to the unwanted modulation frequency. The light adjustment module 115 can change the wavelength of the light wave generated by the light generation module 110 and display device 305 such that the desired modulation frequency is not blocked or attenuated by the unwanted frequency filtering module 120.

NSS Operating with a Tablet

The NSS 105 can operate in conjunction with the tablet 500 as depicted in FIGS. 5A-5D. In some embodiments, the NSS 105 can determine that the visual signaling component 150 hardware includes a tablet device 500 or other display screen that is not affixed or secured to a user's head. The tablet 500 can include a display screen that has one or more component or function of the display screen 305 or light source 305 depicted in conjunction with FIGS. 4A and 4C. The light source 305 in a tablet can be the display screen. The tablet 500 can include one or more feedback sensor that includes one or more component or function of the feedback sensor depicted in conjunction with FIGS. 4B, 4C and 6A.

The tablet 500 can communicate with the NSS 105 via a network, such as a wireless network or a cellular network. The NSS 105 can, in some embodiments, execute the NSS 105 or a component thereof. For example, the tablet 500 can launch, open or switch to an application or resource configured to provide at least one functionality of the NSS 105. The tablet 500 can execute the application as a background process or a foreground process. For example, the graphical user interface for the application can be in the background while the application causes the display screen 305 of the tablet to overlay content or light that changes or modulates at a desired frequency for brain entrainment (e.g., 40 Hz).

The tablet 500 can include one or more feedback sensors 605. In some embodiments, the tablet can use the one or more feedback sensors 605 to detect that a user is holding the tablet 500. The tablet can use the one or more feedback sensors 605 to determine a distance between the light source 305 and the user. The tablet can use the one or more feedback sensors 605 to determine a distance between the light source 305 and the user's head. The tablet can use the one or more feedback sensors 605 to determine a distance between the light source 305 and the user's eyes.

In some embodiments, the tablet 500 can use a feedback sensor 605 that includes a receiver to determine the distance. The tablet can transmit a signal and measure the amount of time it takes for the transmitted signal to leave the tablet 500, bounce on the object (e.g., user's head) and be received by the feedback sensor 605. The tablet 500 or NSS 105 can determine the distance based on the measured amount of time and the speed of the transmitted signal (e.g., speed of light).

In some embodiments, the tablet 500 can include two feedback sensors 605 to determine a distance. The two feedback sensors 605 can include a first feedback sensor 605 that is the transmitter and a second feedback sensor that is the receiver.

In some embodiments, the tablet 500 can include two or more feedback sensors 605 that include two or more cameras. The two or more cameras can measure the angles and the position of the object (e.g., the user's head) on each camera, and use the measured angles and position to determine or compute the distance between the tablet 500 and the object.

In some embodiments, the tablet 500 (or application thereof) can determine the distance between the tablet and the user's head by receiving user input. For example, user input can include an approximate size of the user's head. The tablet 500 can then determine the distance from the user's head based on the inputted approximate size.

The tablet 500, application, or NSS 105 can use the measured or determined distance to adjust the light pulses or flashes of light emitted by the light source 305 of the tablet 500. The tablet 500, application, or NSS 105 can use the distance to adjust one or more parameter of the light pulses, flashes of light or other content emitted via the light source 305 of the tablet 500. For example, the tablet 500 can adjust the intensity of the light pulses emitted by light source 305 based on the distance. The tablet 500 can adjust the intensity based on the distance in order to maintain a consistent or similar intensity at the eye irrespective of the distance between the light source 305 and the eye. The tablet can increase the intensity proportional to the square of the distance.

The tablet 500 can manipulate one or more pixels on the display screen 305 to generate the light pulses or modulation frequency for brainwave entrainment. The tablet 500 can overlay light sources, light pulses or other patterns to generate the modulation frequency for brainwave entrainment. Similar to the virtual reality headset 401, the tablet can filter out or modify unwanted frequencies, wavelengths, or intensities.

Similar to the frames 400, the tablet 500 can adjust a parameter of the light pulses or flashes of light generated by the light source 305 based on ambient light, environmental parameters, or feedback.

In some embodiments, the tablet 500 can execute an application that is configured to generate the light pulses or modulation frequency for brainwave entrainment. The application can execute in the background of the tablet such that all content displayed on a display screen of the tablet are displayed as light pulses at the desired frequency. The tablet can be configured to detect a gaze direction of the user. In some embodiments, the tablet may detect the gaze direction by capturing an image of the user's eye via the camera of the tablet. The tablet 500 can be configured to generate light pulses at particular locations of the display screen based on the gaze direction of the user. In embodiments where direct vision field is to be employed, the light pulses can be displayed at locations of the display screen that correspond to the user's gaze. In embodiments where peripheral vision field is to be employed, the light pulses can be displayed at locations of the displays screen that are outside the portion of the display screen corresponding to the user's gaze.

### Neural Stimulation via Auditory Stimulation

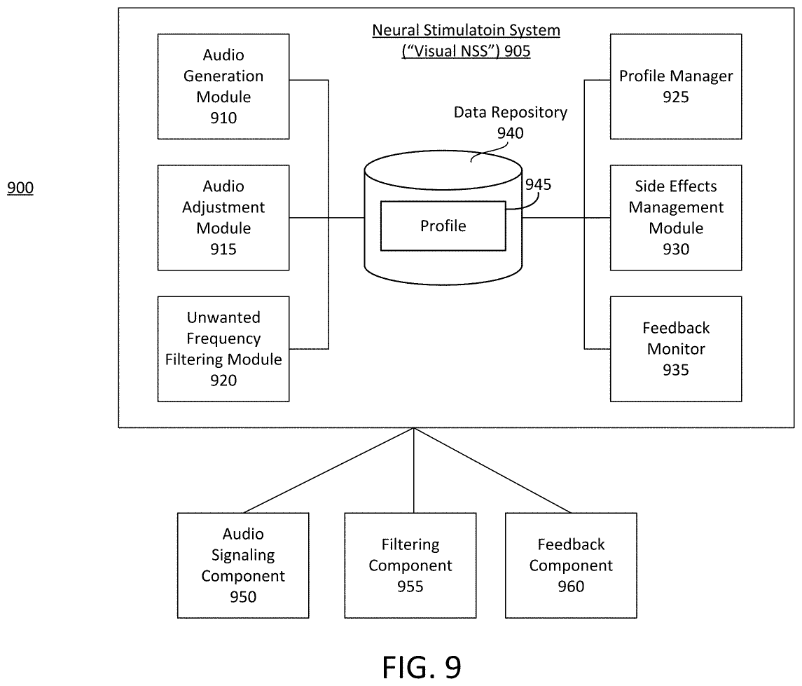

FIG. 9 is a block diagram depicting a system for neural stimulation via auditory stimulation in accordance with an embodiment. The system 900 can include a neural stimulation system (“NSS”) 905. The NSS 905 can be referred to as an auditory NSS 905 or NSS 905. In brief overview, the auditory neural stimulation system (“NSS”) 905 can include, access, interface with, or otherwise communicate with one or more of an audio generation module 910, audio adjustment module 915, unwanted frequency filtering module 920, profile manager 925, side effects management module 930, feedback monitor 935, data repository 940, audio signaling component 950, filtering component 955, or feedback component 960. The audio generation module 910, audio adjustment module 915, unwanted frequency filtering module 920, profile manager 925, side effects management module 930, feedback monitor 935, audio signaling component 950, filtering component 955, or feedback component 960 can each include at least one processing unit or other logic device such as programmable logic array engine, or module configured to communicate with the database repository 950. The audio generation module 910, audio adjustment module 915, unwanted frequency filtering module 920, profile manager 925, side effects management module 930, feedback monitor 935, audio signaling component 950, filtering component 955, or feedback component 960 can be separate components, a single component, or part of the NSS 905. The system 100 and its components, such as the NSS 905, may include hardware elements, such as one or more processors, logic devices, or circuits. The system 100 and its components, such as the NSS 905, can include one or more hardware or interface component depicted in system 700 in FIGS. 7A and 7B. For example, a component of system 100 can include or execute on one or more processors 721, access storage 728 or memory 722, and communicate via network interface 718.

Still referring to FIG. 9, and in further detail, the NSS 905 can include at least one audio generation module 910. The audio generation module 910 can be designed and constructed to interface with an audio signaling component 950 to provide instructions or otherwise cause or facilitate the generation of an audio signal, such as an audio burst, audio pulse, audio chirp, audio sweep, or other acoustic wave having one or more predetermined parameters. The audio generation module 910 can include hardware or software to receive and process instructions or data packets from one or more module or component of the NSS 905. The audio generation module 910 can generate instructions to cause the audio signaling component 950 to generate an audio signal. The audio generation module 910 can control or enable the audio signaling component 950 to generate the audio signal having one or more predetermined parameters.

The audio generation module 910 can be communicatively coupled to the audio signaling component 950. The audio generation module 910 can communicate with the audio signaling component 950 via a circuit, electrical wire, data port, network port, power wire, ground, electrical contacts or pins. The audio generation module 910 can wirelessly communicate with the audio signaling component 950 using one or more wireless protocols such as BlueTooth, BlueTooth Low Energy, Zigbee, Z-Wave, IEEE 802, WIFI, 3G, 4G, LTE, near field communications (“NFC”), or other short, medium or long range communication protocols, etc. The audio generation module 910 can include or access network interface 718 to communicate wirelessly or over a wire with the audio signaling component 950.

The audio generation module 910 can interface, control, or otherwise manage various types of audio signaling components 950 in order to cause the audio signaling component 950 to generate, block, control, or otherwise provide the audio signal having one or more predetermined parameters. The audio generation module 910 can include a driver configured to drive an audio source of the audio signaling component 950. For example, the audio source can include a speaker, and the audio generation module 910 (or the audio signaling component) can include a transducer that converts electrical energy to sound waves or acoustic waves. The audio generation module 910 can include a computing chip, microchip, circuit, microcontroller, operational amplifiers, transistors, resistors, or diodes configured to provide electricity or power having certain voltage and current characteristics to drive the speaker to generate an audio signal with desired acoustic characteristics.

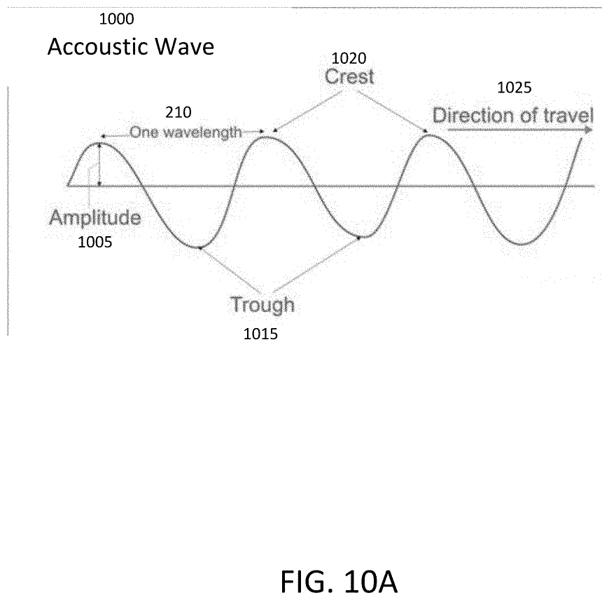

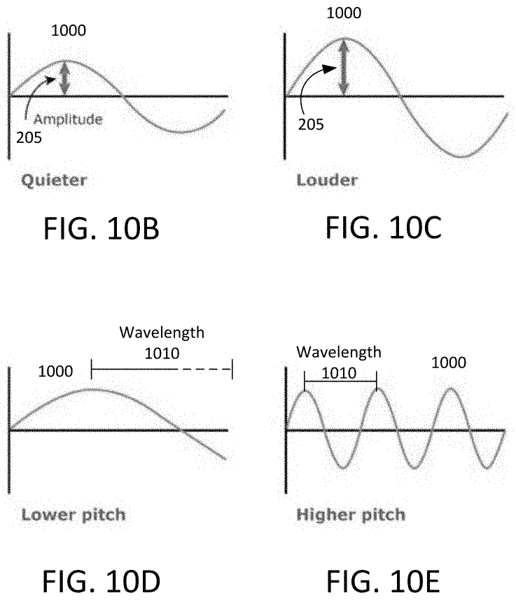

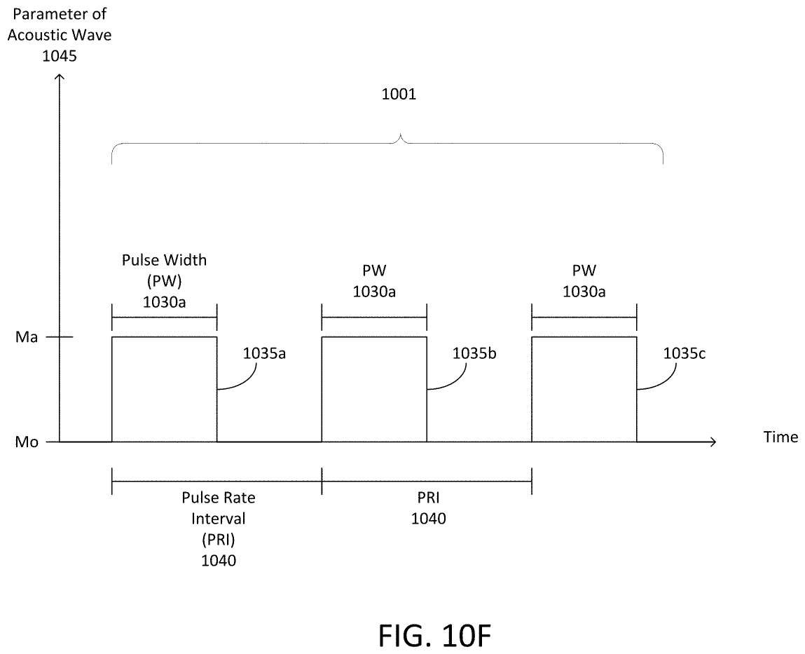

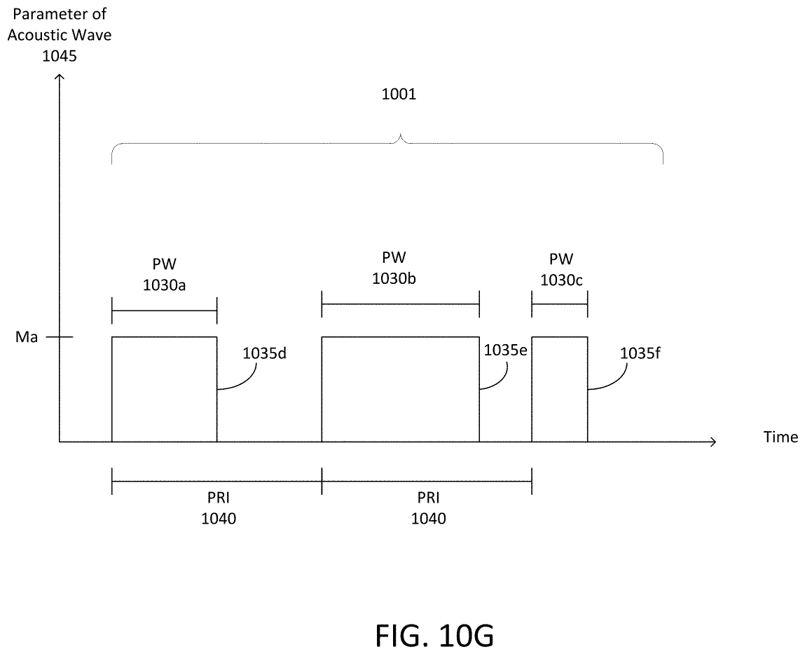

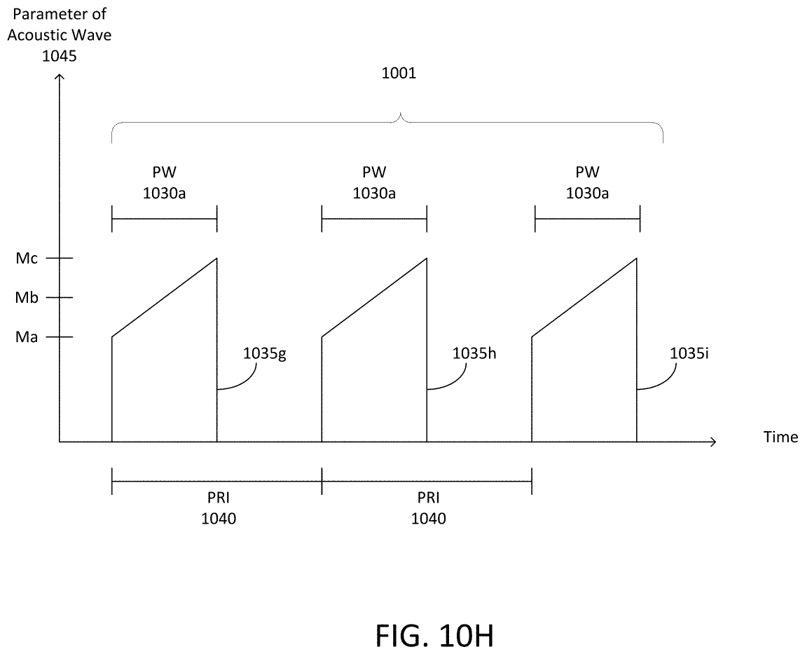

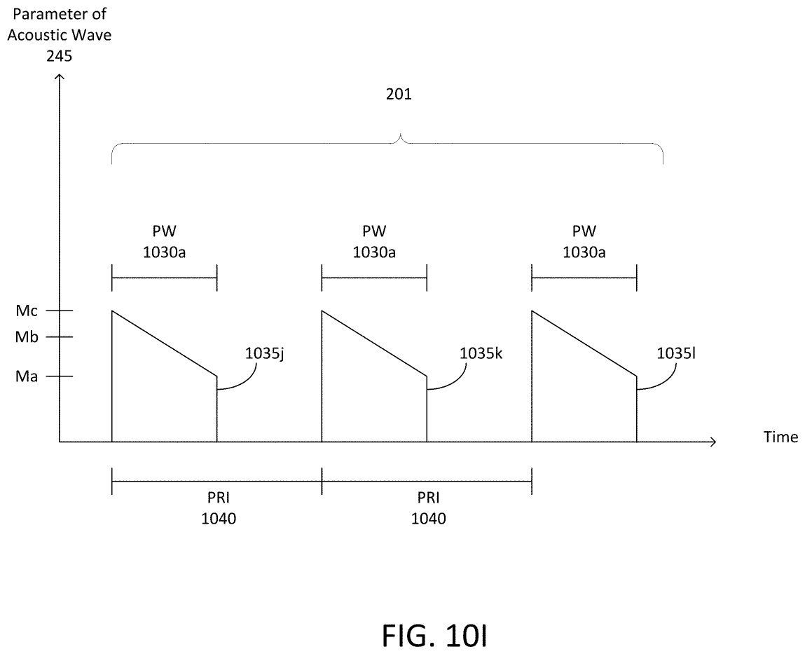

In some embodiments, the audio generation module 910 can instruct the audio signaling component 950 to provide an audio signal. For example, the audio signal can include an acoustic wave 1000 as depicted in FIG. 10A. The audio signal can include multiple acoustic waves. The audio signal can generate one or more acoustic waves. The acoustic wave 1000 can include or be formed of a mechanical wave of pressure and displacement that travels through media such as gases, liquids, and solids. The acoustic wave can travel through a medium to cause vibration, sound, ultrasound or infrasound. The acoustic wave can propagate through air, water or solids as longitudinal waves. The acoustic wave can propagate through solids as a transverse wave.

The acoustic wave can generate sound due to the oscillation in pressure, stress, particle displacement, or particle velocity propagated in a medium with internal forces (e.g., elastic or viscous), or the superposition of such propagated oscillation. Sound can refer to the auditory sensation evoked by this oscillation. For example, sound can refer to the reception of acoustic waves and their perception by the brain.

The audio signaling component 950 or audio source thereof can generate the acoustic waves by vibrating a diaphragm of the audio source. For example, the audio source can include a diaphragm such as a transducer configured to inter-convert mechanical vibrations to sounds. The diaphragm can include a thin membrane or sheet of various materials, suspended at its edges. The varying pressure of sound waves imparts mechanical vibrations to the diaphragm which can then create acoustic waves or sound.

The acoustic wave 1000 illustrated in FIG. 10A includes a wavelength 1010. The wavelength 1010 can refer to a distance between successive crests 1020 of the wave. The wavelength 1010 can be related to the frequency of the acoustic wave and the speed of the acoustic wave. For example, the wavelength can be determined as the quotient of the speed of the acoustic wave divided by the frequency of the acoustic wave. The speed of the acoustic wave can the product of the frequency and the wavelength. The frequency of the acoustic wave can be the quotient of the speed of the acoustic wave divided by the wavelength of the acoustic wave. Thus, the frequency and the wavelength of the acoustic wave can be inversely proportional. The speed of sound can vary based on the medium through which the acoustic wave propagates. For example, the speed of sound in air can be 343 meters per second.

A crest 1020 can refer to the top of the wave or point on the wave with the maximum value. The displacement of the medium is at a maximum at the crest 1020 of the wave. The trough 1015 is the opposite of the crest 1020. The trough 1015 is the minimum or lowest point on the wave corresponding to the minimum amount of displacement.

The acoustic wave 1000 can include an amplitude 1005. The amplitude 1005 can refer to a maximum extent of a vibration or oscillation of the acoustic wave 1000 measured from a position of equilibrium. The acoustic wave 1000 can be a longitudinal wave if it oscillates or vibrates in the same direction of travel 1025. In some cases, the acoustic wave 1000 can be a transverse wave that vibrates at right angles to the direction of its propagation.

The audio generation module 910 can instruct the audio signaling component 950 to generate acoustic waves or sound waves having one or more predetermined amplitude or wavelength. Wavelengths of the acoustic wave that are audible to the human ear range from approximately 17 meters to 17 millimeters (or 20 Hz to 20 kHz). The audio generation module 910 can further specify one or more properties of an acoustic wave within or outside the audible spectrum. For example, the frequency of the acoustic wave can range from 0 to 50 kHz. In some embodiments, the frequency of the acoustic wave can range from 8 to 12 kHz. In some embodiments, the frequency of the acoustic wave can be 10 kHz.

The NSS 905 can modulate, modify, change or otherwise alter properties of the acoustic wave 1000. For example, the NSS 905 can modulate the amplitude or wavelength of the acoustic wave. As depicted in FIG. 10B and FIG. 10C, the NSS 905 can adjust, manipulate, or otherwise modify the amplitude 1005 of the acoustic wave 1000. For example, the NSS 905 can lower the amplitude 1005 to cause the sound to be quieter, as depicted in FIG. 10B, or increase the amplitude 1005 to cause the sound to be louder, as depicted in FIG. 10C.

In some cases, the NSS 905 can adjust, manipulate or otherwise modify the wavelength 1010 of the acoustic wave. As depicted in FIG. 10D and FIG. 10E, the NSS 905 can adjust, manipulate, or otherwise modify the wavelength 1010 of the acoustic wave 1000. For example, the NSS 905 can increase the wavelength 1010 to cause the sound to have a lower pitch, as depicted in FIG. 10D, or reduce the wavelength 1010 to cause the sound to have a higher pitch, as depicted in FIG. 10E.

The NSS 905 can modulate the acoustic wave. Modulating the acoustic wave can include modulating one or more properties of the acoustic wave. Modulating the acoustic wave can include filtering the acoustic wave, such as filtering out unwanted frequencies or attenuating the acoustic wave to lower the amplitude. Modulating the acoustic wave can include adding one or more additional acoustic waves to the original acoustic wave. Modulating the acoustic wave can include combining the acoustic wave such that there is constructive or destructive interference where the resultant, combined acoustic wave corresponds to the modulated acoustic wave.

The NSS 905 can modulate or change one or more properties of the acoustic wave based on a time interval. The NSS 905 can change the one or more properties of the acoustic at the end of the time interval. For example, the NSS 905 can change a property of the acoustic wave every 30 seconds, 1 minute, 2 minutes, 3 minutes, 5 minutes, 7 minutes, 10 minutes, or 15 minutes. The NSS 905 can change a modulation frequency of the acoustic wave, where the modulation frequency refers to the repeated modulations or inverse of the pulse rate interval of the acoustic pulses. The modulation frequency can be a predetermined or desired frequency. The modulation frequency can correspond to a desired stimulation frequency of neural oscillations. The modulation frequency can be set to facilitate or cause brainwave entrainment. The NSS 905 can set the modulation frequency to a frequency in the range of 0.1 Hz to 10,000 Hz. For example, the NSS 905 can set the modulation frequency to 0.1 Hz, 1 Hz, 5 Hz, 10 Hz, 20 Hz, 25 Hz, 30 Hz, 31 Hz, 32 Hz, 33 Hz, 34 Hz, 35 Hz, 36 Hz, 37 Hz, 38 Hz, 39 Hz, 40 Hz, 41 Hz, 42 Hz, 43 Hz, 44 Hz, 45 Hz, 46 Hz, 47 Hz, 48 Hz, 49 Hz, 50 Hz, 60 Hz, 70 Hz, 80 Hz, 90 Hz, 100 Hz, 150 Hz, 200 Hz, 250 Hz, 300 Hz, 400 Hz, 500 Hz, 1000 Hz, 2000 Hz, 3000 Hz, 4,000 Hz, 5000 Hz, 6,000 Hz, 7,000 Hz, 8,000 Hz, 9,000 Hz, or 10,000 Hz.

The audio generation module 910 can determine to provide audio signals that include bursts of acoustic waves, audio pulses, or modulations to acoustic waves. The audio generation module 910 can instruct or otherwise cause the audio signaling component 950 to generate acoustic bursts or pulses. An acoustic pulse can refer to a burst of acoustic waves or a modulation to a property of an acoustic wave that is perceived by the brain as a change in sound. For example, an audio source that is intermittently turned on and off can create audio bursts or changes in sound. The audio source can be turned on and off based on a predetermined or fixed pulse rate interval, such as every 0.025 seconds, to provide a pulse repetition frequency of 40 Hz. The audio source can be turned on and off to provide a pulse repetition frequency in the range of 0.1 Hz to 10 kHz or more.

For example, FIGS. 10F-10I illustrates bursts of acoustic waves or bursts of modulations that can be applied to acoustic waves. The bursts of acoustic waves can include, for example, audio tones, beeps, or clicks. The modulations can refer to changes in the amplitude of the acoustic wave, changes in frequency or wavelength of the acoustic wave, overlaying another acoustic wave over the original acoustic wave, or otherwise modifying or changing the acoustic wave.

For example, FIG. 10F illustrates acoustic bursts 1035a-c (or modulation pulses 1035a-c) in accordance with an embodiment. The acoustic bursts 1035a-c can be illustrated via a graph where the y-axis represents a parameter of the acoustic wave (e.g., frequency, wavelength, or amplitude) of the acoustic wave. The x-axis can represent time (e.g., seconds, milliseconds, or microseconds).

The audio signal can include a modulated acoustic wave that is modulated between different frequencies, wavelengths, or amplitudes. For example, the NSS 905 can modulate an acoustic wave between a frequency in the audio spectrum, such as Ma, and a frequency outside the audio spectrum, such as Mo. The NSS 905 can modulate the acoustic wave between two or more frequencies, between an on state and an off state, or between a high power state and a low power state.

The acoustic bursts 1035a-c can have an acoustic wave parameter with value Ma that is different from the value Mo of the acoustic wave parameter. The modulation Ma can refer to a frequency or wavelength, or amplitude. The pulses 1035a-c can be generated with a pulse rate interval (PRI) 1040.

For example, the acoustic wave parameter can be the frequency of the acoustic wave. The first value Mo can be a low frequency or carrier frequency of the acoustic wave, such as 10 kHz. The second value, Ma, can be different from the first frequency Mo. The second frequency Ma can be lower or higher than the first frequency Mo. For example, the second frequency Ma can be 11 kHz. The difference between the first frequency and the second frequency can be determined or set based on a level of sensitivity of the human ear. The difference between the first frequency and the second frequency can be determined or set based on profile information 945 for the subject. The difference between the first frequency Mo and the second frequency Ma can be determined such that the modulation or change in the acoustic wave facilitate brainwave entrainment.

In some cases, the parameter of the acoustic wave used to generate the acoustic burst 1035a can be constant at Ma, thereby generating a square wave as illustrated in FIG. 10F. In some embodiments, each of the three pulses 1035a-c can include acoustic waves having a same frequency Ma.

The width of each of the acoustic bursts or pulses (e.g., the duration of the burst of the acoustic wave with the parameter Ma) can correspond to a pulse width 1030a. The pulse width 1030a can refer to the length or duration of the burst. The pulse width 1030a can be measured in units of time or distance. In some embodiments, the pulses 1035a-c can include acoustic waves having different frequencies from one another. In some embodiments, the pulses 1035a-c can have different pulse widths 1030a from one another, as illustrated in FIG. 10G. For example, a first pulse 1035d of FIG. 10G can have a pulse width 1030a, while a second pulse 1035e has a second pulse width 1030b that is greater than the first pulse width 1030a. A third pulse 1035f can have a third pulse width 1030c that is less than the second pulse width 1030b. The third pulse width 1030c can also be less than the first pulse width 1030a. While the pulse widths 1030a-c of the pulses 1035d-f of the pulse train may vary, the audio generation module 910 can maintain a constant pulse rate interval 1040 for the pulse train.

The pulses 1035a-c can form a pulse train having a pulse rate interval 1040. The pulse rate interval 1040 can be quantified using units of time. The pulse rate interval 1040 can be based on a frequency of the pulses of the pulse train 201. The frequency of the pulses of the pulse train 201 can be referred to as a modulation frequency. For example, the audio generation module 910 can provide a pulse train 201 with a predetermined frequency, such as 40 Hz. To do so, the audio generation module 910 can determine the pulse rate interval 1040 by taking the multiplicative inverse (or reciprocal) of the frequency (e.g., 1 divided by the predetermined frequency for the pulse train). For example, the audio generation module 910 can take the multiplicative inverse of 40 Hz by dividing 1 by 40 Hz to determine the pulse rate interval 1040 as 0.025 seconds. The pulse rate interval 1040 can remain constant throughout the pulse train. In some embodiments, the pulse rate interval 1040 can vary throughout the pulse train or from one pulse train to a subsequent pulse train. In some embodiments, the number of pulses transmitted during a second can be fixed, while the pulse rate interval 1040 varies.

In some embodiments, the audio generation module 910 can generate an audio burst or audio pulse having an acoustic wave that varies in frequency, amplitude, or wavelength. For example, the audio generation module 910 can generate up-chirp pulses where the frequency, amplitude or wavelength of the acoustic wave of the audio pulse increases from the beginning of the pulse to the end of the pulse as illustrated in FIG. 10H. For example, the frequency, amplitude or wavelength of the acoustic wave at the beginning of pulse 1035g can be Ma. The frequency, amplitude or wavelength of the acoustic wave of the pulse 1035g can increase from Ma to Mb in the middle of the pulse 1035g, and then to a maximum of Mc at the end of the pulse 1035g. Thus, the frequency, amplitude or wavelength of the acoustic wave used to generate the pulse 1035g can range from Ma to Mc. The frequency, amplitude or wavelength can increase linearly, exponentially, or based on some other rate or curve. One or more of the frequency, amplitude, or wavelength of the acoustic wave can change from the beginning of the pulse to the end of the pulse.

The audio generation module 910 can generate down-chirp pulses, as illustrated in FIG. 10I, where the frequency, amplitude or wavelength of the acoustic wave of the acoustic pulse decreases from the beginning of the pulse to the end of the pulse. For example, the frequency, amplitude or wavelength of an acoustic wave at the beginning of pulse 1035j can be Mc. The frequency, amplitude or wavelength of the acoustic wave of the pulse 1035j can decrease from Mc to Mb in the middle of the pulse 1035j, and then to a minimum of Ma at the end of the pulse 1035j. Thus, the frequency, amplitude or wavelength of the acoustic wave used to generate the pulse 1035j can range from Mc to Ma. The frequency, amplitude or wavelength can decrease linearly, exponentially, or based on some other rate or curve. One or more of the frequency, amplitude or wavelength of the acoustic wave can change from the beginning of the pulse to the end of the pulse.

In some embodiments, the audio generation module 910 can instruct or cause the audio signaling component 950 to generate audio pulses to stimulate specific or predetermined portions of the brain or a specific cortex. The frequency, wavelength, modulation frequency, amplitude and other aspects of the audio pulse, tone or music based stimuli can dictate which cortex or cortices are recruited to process the stimuli. The audio signaling component 950 can stimulate discrete portions of the cortex by modulating the presentation of the stimuli to target specific or general regions of interest. The modulation parameters or amplitude of the audio stimuli can dictate which region of the cortex is stimulated. For example, different regions of the cortex are recruited to process different frequencies of sound, called their characteristic frequencies. Further, ear laterality of stimulation can have an effect on cortex response since some subjects can be treated by stimulating one ear as opposed to both ears.

Audio signaling component 950 can be designed and constructed to generate the audio pulses responsive to instructions from the audio generation module 910. The instructions can include, for example, parameters of the audio pulse such as a frequency, wavelength or of the acoustic wave, duration of the pulse, frequency of the pulse train, pulse rate interval, or duration of the pulse train (e.g., a number of pulses in the pulse train or the length of time to transmit a pulse train having a predetermined frequency). The audio pulse can be perceived, observed, or otherwise identified by the brain via cochlear means such as ears. The audio pulses can be transmitted to the ear via an audio source speaker in close proximity to the ear, such as headphones, earbuds, bone conduction transducers, or cochlear implants. The audio pulses can be transmitted to the ear via an audio source or speaker not in close proximity to the ear, such as a surround sound speaker system, bookshelf speakers, or other speaker not directly or indirectly in contact with the ear.

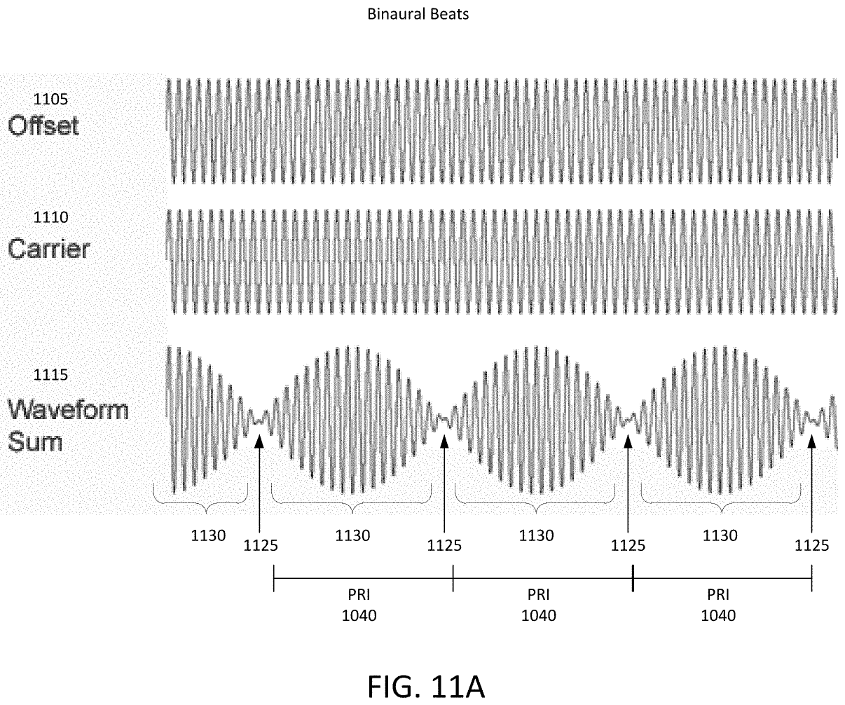

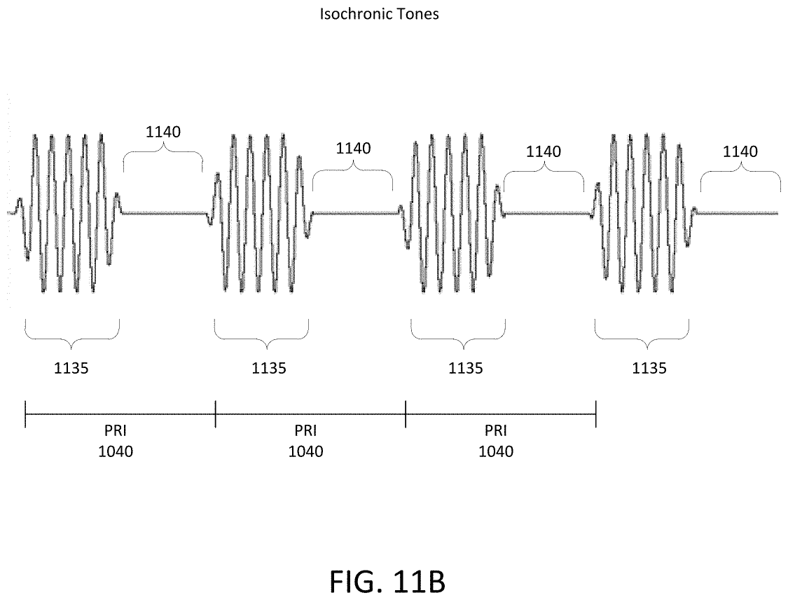

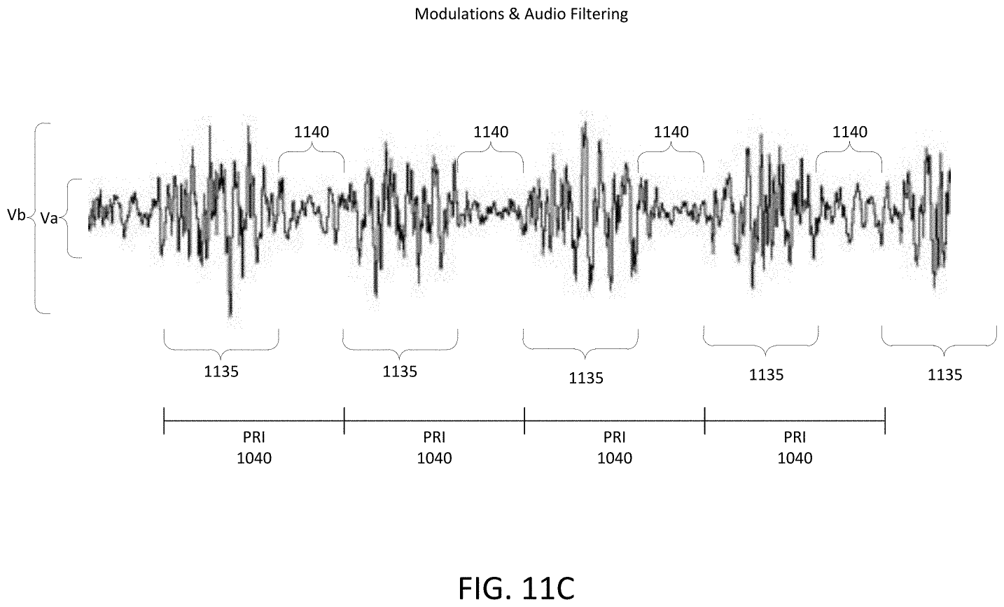

FIG. 11A illustrates audio signals using binaural beats or binaural pulses, in accordance with an embodiment. In brief summary, binaural beats refers to providing a different tone to each ear of the subject. When the brain perceives the two different tones, the brain mixes the two tones together to create a pulse. The two different tones can be selected such that the sum of the tones creates a pulse train having a desired pulse rate interval 1040.

The audio signaling component 950 can include a first audio source that provides an audio signal to the first ear of a subject, and a second audio source that provides a second audio signal to the second ear of a subject. The first audio source and the second audio source can be different. The first ear may only perceive the first audio signal from the first audio source, and the second ear may only receive the second audio signal from the second audio source. Audio sources can include, for example, headphones, earbuds, or bone conduction transducers. The audio sources can include stereo audio sources.

The audio generation component 910 can select a first tone for the first ear and a different second tone for the second ear. A tone can be characterized by its duration, pitch, intensity (or loudness), or timbre (or quality). In some cases, the first tone and the second tone can be different if they have different frequencies. In some cases, the first tone and the second tone can be different if they have different phase offsets. The first tone and the second tone can each be pure tones. A pure tone can be a tone having a sinusoidal waveform with a single frequency.

As illustrated in FIG. 11A, the first tone or offset wave 1105 is slightly different from the second tone 1110 or carrier wave 1110. The first tone 1105 has a higher frequency than the second tone 1110. The first tone 1105 can be generated by a first earbud that is inserted into one of the subject's ears, and the second tone 1110 can be generated by a second earbud that is inserted into the other of the subject's ears. When the auditory cortex of the brain perceives the first tone 1105 and the second tone 1110, the brain can sum the two tones. The brain can sum the acoustic waveforms corresponding to the two tones. The brain can sum the two waveforms as illustrated by waveform sum 1115. Due to the first and second tones having a different parameter (such as a different frequency or phase offset), portions of the waves can add and subtract from another to result in waveform 1115 having one or more pulses 1130 (or beats 1130). The pulses 1130 can be separated by portions 1125 that are at equilibrium. The pulses 1130 perceived by the brain by mixing these two different waveforms together can induce brainwave entrainment.

In some embodiments, the NSS 905 can generate binaural beats using a pitch panning technique. For example, the audio generation module 910 or audio adjustment module 915 can include or use a filter to modulate the pitch of a sound file or single tone up and down, and at the same time pan the modulation between stereo sides, such that one side will have a slightly higher pitch while the other side has a pitch that is slightly lower. The stereo sides can refer to the first audio source that generates and provides the audio signal to the first ear of the subject, and the second audio source that generates and provides the audio signal to the second ear of the subject. A sound file can refer to a file format configured to store a representation of, or information about, an acoustic wave. Example sound file formats can include .mp3, .wav, .aac, .m4a, .smf, etc.

The NSS 905 can use this pitch panning technique to generate a type of spatial positioning that, when listened to through stereo headphones, is perceived by the brain in a manner similar to binaural beats. The NSS 905 can, therefore, use this pitch panning technique to generate pulses or beats using a single tone or a single sound file.

In some cases, the NSS 905 can generate monaural beats or monaural pulses. Monaural beats or pulses are similar to binaural beats in that they are also generated by combining two tones to form a beat. The NSS 905 or component of system 100 can form monaural beats by combining the two tones using a digital or analog technique before the sound reaches the ears, as opposed to the brain combining the waveforms as in binaural beats. For example, the NSS 905 (or audio generation component 910) can identify and select two different waveforms that, when combined, produce beats or pulses having a desired pulse rate interval. The NSS 905 can identify a first digital representation of a first acoustic waveform, and identify a second digital representation of a second acoustic waveform have a different parameter than the first acoustic waveform. The NSS 905 can combine the first and second digital waveforms to generate a third digital waveform different from the first digital waveform and the second digital waveform. The NSS 905 can then transmit the third digital waveform in a digital form to the audio signaling component 950. The NSS 905 can translate the digital waveform to an analog format and transmit the analog format to the audio signaling component 950. The audio signaling component 950 can then, via an audio source, generate the sound to be perceived by one or both ears. The same sound can be perceived by both ears. The sound can include the pulses or beats spaced at the desired pulse rate interval 1040.

FIG. 11B illustrates acoustic pulses having isochronic tones, in accordance with an embodiment. Isochronic tones are evenly spaced tone pulses. Isochronic tones can be created without having to combine two different tones. The NSS 905 or other component of system 100 can create the isochronic tone by turning a tone on and off. The NSS 905 can generate the isochronic tones or pulses by instructing the audio signaling component to turn on and off The NSS 905 can modify a digital representation of an acoustic wave to remove or set digital values of the acoustic wave such that sound is generated during the pulses 1135 and no sound is generated during the null portions 1140.

By turning on and off the acoustic wave, the NSS 905 can establish acoustic pulses 1135 that are spaced apart by a pulse rate interval 1040 that corresponds to a desired stimulation frequency, such as 40 Hz. The isochronic pulses spaced part at the desired PRI 1040 can induce brainwave entrainment.

FIG. 11C illustrates audio pulses generated by the NSS 905 using a sound track, in accordance with an embodiment. A sound track can include or refer to a complex acoustical wave that includes multiple different frequencies, amplitudes, or tones. For example, a sound track can include a voice track, a musical instrument track, a musical track having both voice and musical instruments, nature sounds, or white noise.

The NSS 905 can modulate the sound track to induce brainwave entrainment by rhythmically adjusting a component in the sound. For example, the NSS 905 can modulate the volume by increasing and decreasing the amplitude of the acoustic wave or sound track to create the rhythmic stimulus corresponding to the stimulation frequency for inducing brainwave entrainment. Thus, the NSS 905 can embed, into a sound track acoustic pulses having a pulse rate interval corresponding to the desired stimulation frequency to induce brainwave entrainment. The NSS 905 can manipulate the sound track to generate a new, modified sound track having acoustic pulses with a pulse rate interval corresponding to the desired stimulation frequency to induce brainwave entrainment.

As illustrated in FIG. 11C, pulses 1135 are generated by modulating the volume from a first level Va to a second level Vb. During portions 1140 of the acoustic wave 345, the NSS 905 can set or keep the volume at Va. The volume Va can refer to an amplitude of the wave, or a maximum amplitude or crest of the wave 345 during the portion 1140. The NSS 905 can then adjust, change, or increase the volume to Vb during portion 1135. The NSS 905 can increase the volume by a predetermined amount, such as a percentage, a number of decibels, a subject-specified amount, or other amount. The NSS 905 can set or maintain the volume at Vb for a duration corresponding to a desired pulse length for the pulse 1135.

In some embodiments, the NSS 905 can include an attenuator to attenuate the volume from level Vb to level Va. In some embodiments, the NSS 905 can instruct an attenuator (e.g., an attenuator of audio signaling component 950) to attenuate the volume from level Vb to level Va. In some embodiments, the NSS 905 can include an amplifier to amplify or increase the volume from Va to Vb. In some embodiments, the NSS 905 can instruct an amplifier (e.g., an amplifier of the audio signaling component 950) to amplify or increase the volume from Va to Vb.

Referring back to FIG. 9, the NSS 905 can include, access, interface with, or otherwise communicate with at least one audio adjustment module 915. The audio adjustment module 915 can be designed and constructed to adjust a parameter associated with the audio signal, such as a frequency, amplitude, wavelength, pattern or other parameter of the audio signal. The audio adjustment module 915 can automatically vary a parameter of the audio signal based on profile information or feedback. The audio adjustment module 915 can receive the feedback information from the feedback monitor 935. The audio adjustment module 915 can receive instructions or information from a side effects management module 930. The audio adjustment module 915 can receive profile information from profile manager 925.

The NSS 905 can include, access, interface with, or otherwise communicate with at least one unwanted frequency filtering module 920. The unwanted frequency filtering module 920 can be designed and constructed to block, mitigate, reduce, or otherwise filter out frequencies of audio signals that are undesired to prevent or reduce an amount of such audio signals from being perceived by the brain. The unwanted frequency filtering module 920 can interface, instruct, control, or otherwise communicate with a filtering component 955 to cause the filtering component 955 to block, attenuate, or otherwise reduce the effect of the unwanted frequency on the neural oscillations.

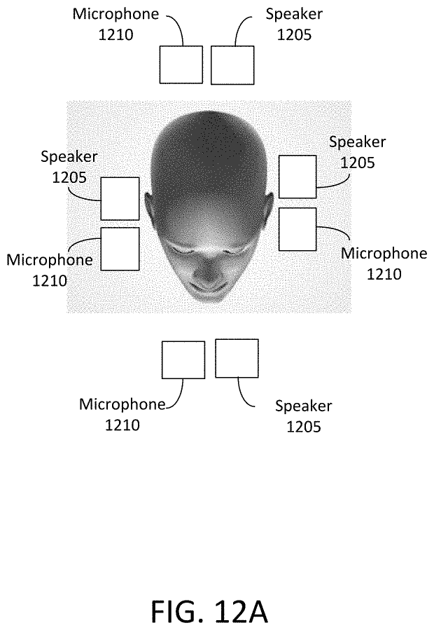

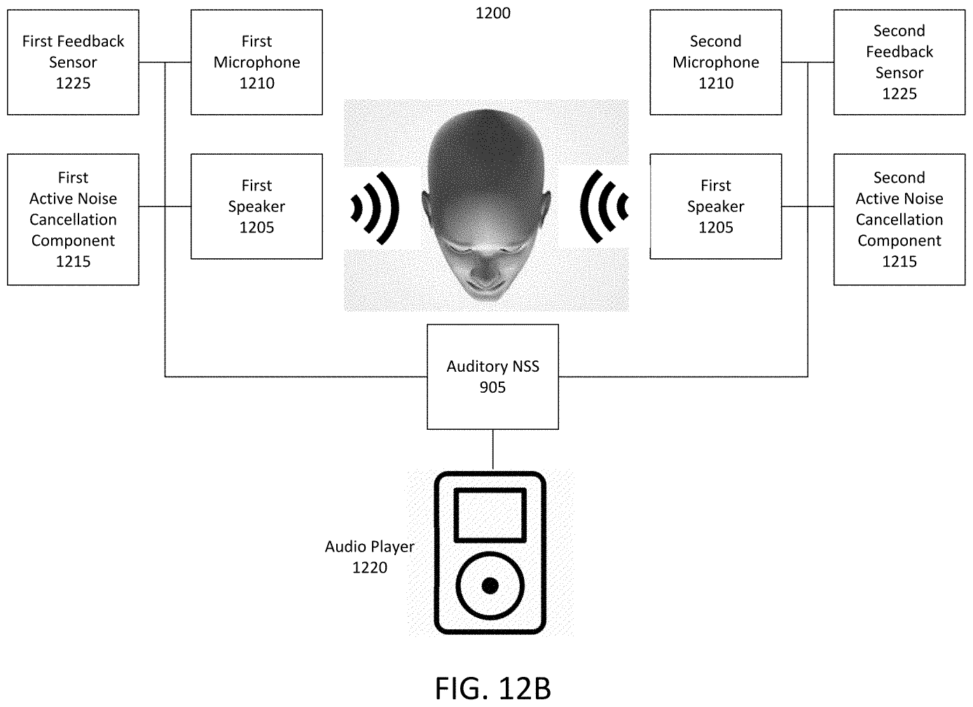

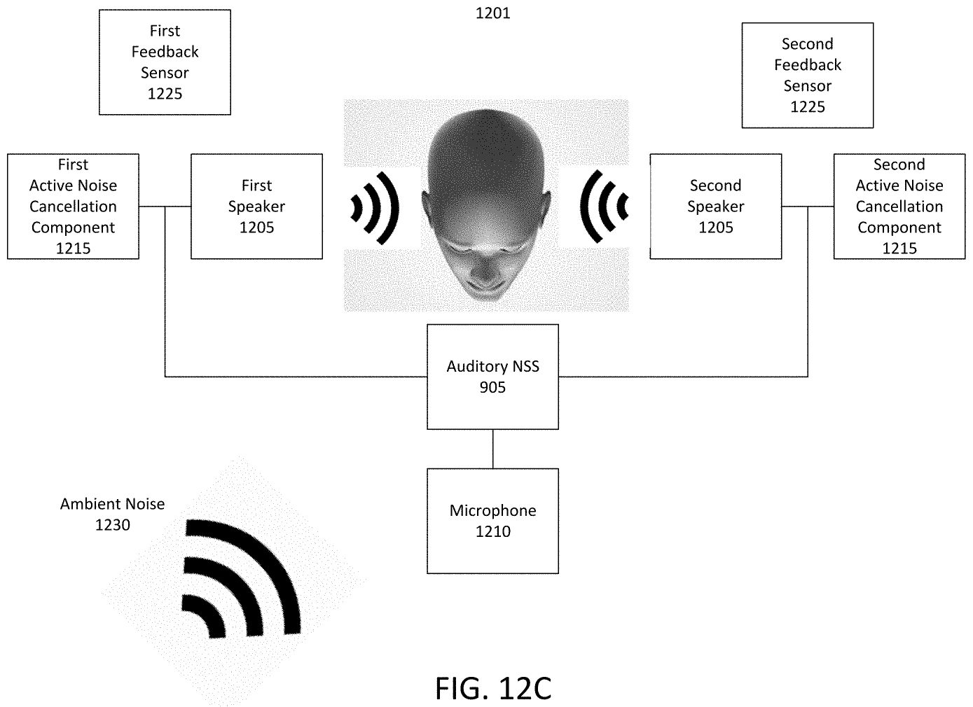

The unwanted frequency filtering module 920 can include an active noise control component (e.g., active noise cancellation component 1215 depicted in FIG. 12B). Active noise control can be referred to or include active noise cancellation or active noise reduction. Active noise control can reduce an unwanted sound by adding a second sound having a parameter specifically selected to cancel or attenuate the first sound. In some cases, the active noise control component can emit a sound wave with the same amplitude but with an inverted phase (or antiphase) to the original unwanted sound. The two waves can combine to form a new wave, and effectively cancel each other out by destructive interference.

The active noise control component can include analog circuits or digital signal processing. The active noise control component can include adaptive techniques to analyze waveforms of the background aural or nonaural noise. Responsive to the background noise, the active noise control component can generate an audio signal that can either phase shift or invert the polarity of the original signal. This inverted signal can be amplified by a transducer or speaker to create a sound wave directly proportional to the amplitude of the original waveform, creating destructive interference. This can reduce the volume of the perceivable noise.

In some embodiments, a noise-cancellation speaker can be co-located with a sound source speaker. In some embodiments, a noise cancellation speaker can be co-located with a sound source that is to be attenuated.

The unwanted frequency filtering module 920 can filter out unwanted frequencies that can adversely impact auditory brainwave entrainment. For example, an active noise control component can identify that audio signals include acoustic bursts having the desired pulse rate interval, as well as acoustic bursts having an unwanted pulse rate interval. The active noise control component can identify the waveforms corresponding to the acoustic bursts having the unwanted pulse rate interval, and generate an inverted phase waveform to cancel out or attenuate the unwanted acoustic bursts.

The NSS 905 can include, access, interface with, or otherwise communicate with at least one profile manager 925. The profile manager 925 can be designed or constructed to store, update, retrieve or otherwise manage information associated with one or more subjects associated with the auditory brain entrainment. Profile information can include, for example, historical treatment information, historical brain entrainment information, dosing information, parameters of acoustic waves, feedback, physiological information, environmental information, or other data associated with the systems and methods of brain entrainment.

The NSS 905 can include, access, interface with, or otherwise communicate with at least one side effects management module 930. The side effects management module 930 can be designed and constructed to provide information to the audio adjustment module 915 or the audio generation module 910 to change one or more parameter of the audio signal in order to reduce a side effect. Side effects can include, for example, nausea, migraines, fatigue, seizures, ear strain, deafness, ringing, or tinnitus.

The side effects management module 930 can automatically instruct a component of the NSS 905 to alter or change a parameter of the audio signal. The side effects management module 930 can be configured with predetermined thresholds to reduce side effects. For example, the side effects management module 930 can be configured with a maximum duration of a pulse train, maximum amplitude of acoustic waves, maximum volume, maximum duty cycle of a pulse train (e.g., the pulse width multiplied by the frequency of the pulse train), maximum number of treatments for brainwave entrainment in a time period (e.g., 1 hour, 2 hours, 12 hours, or 24 hours).

The side effects management module 930 can cause a change in the parameter of the audio signal in response to feedback information. The side effect management module 930 can receive feedback from the feedback monitor 935. The side effects management module 930 can determine to adjust a parameter of the audio signal based on the feedback. The side effects management module 930 can compare the feedback with a threshold to determine to adjust the parameter of the audio signal.

The side effects management module 930 can be configured with or include a policy engine that applies a policy or a rule to the current audio signal and feedback to determine an adjustment to the audio signal. For example, if feedback indicates that a patient receiving audio signals has a heart rate or pulse rate above a threshold, the side effects management module 930 can turn off the pulse train until the pulse rate stabilizes to a value below the threshold, or below a second threshold that is lower than the threshold.

The NSS 905 can include, access, interface with, or otherwise communicate with at least one feedback monitor 935. The feedback monitor can be designed and constructed to receive feedback information from a feedback component 960. Feedback component 960 can include, for example, a feedback sensor 1405 such as a temperature sensor, heart or pulse rate monitor, physiological sensor, ambient noise sensor, microphone, ambient temperature sensor, blood pressure monitor, brain wave sensor, EEG probe, electrooculography (“EOG”) probes configured measure the corneo-retinal standing potential that exists between the front and the back of the human eye, accelerometer, gyroscope, motion detector, proximity sensor, camera, microphone, or photo detector.

Systems and Devices Configured for Neural Stimulation via Auditory Stimulation

FIG. 12A illustrates a system for auditory brain entrainment in accordance with an embodiment. The system 1200 can include one or more speakers 1205. The system 1200 can include one or more microphones. In some embodiments, the system can include both speakers 1205 and microphones 1210. In some embodiments, the system 1200 includes speakers 1205 and may not include microphones 1210. In some embodiments, the system 1200 includes microphones 1210 and may not include speakers 1210.

The speakers 1205 can be integrated with the audio signaling component 950. The audio signaling component 950 can include speakers 1205. The speakers 1205 can interact or communicate with audio signaling component 950. For example, the audio signaling component 950 can instruct the speaker 1205 to generate sound.

The microphones 1210 can be integrated with the feedback component 960. The feedback component 960 can include microphones 1210. The microphones 1210 can interact or communicate with feedback component 960. For example, the feedback component 960 can receive information, data or signals from microphone 1210.

In some embodiments, the speaker 1205 and the microphone 1210 can be integrated together or a same device. For example, the speaker 1205 can be configured to function as the microphone 1210. The NSS 905 can toggle the speaker 1205 from a speaker mode to a microphone mode.

In some embodiments, the system 1200 can include a single speaker 1205 positioned at one of the ears of the subject. In some embodiments, the system 1200 can include two speakers. A first speaker of the two speakers can be positioned at a first ear, and the second speaker of the two speakers can be positioned at the second ear. In some embodiments, additional speakers can be positioned in front of the subject's head, or behind the subject's head. In some embodiments, one or more microphones 1210 can be positioned at one or both ears, in front of the subject's head, or behind the subject's head.

The speaker 1205 can include a dynamic cone speaker configured to produce sound from an electrical signal. The speaker 1205 can include a full-range driver to produce acoustic waves with frequencies over some or all of the audible range (e.g., 60 Hz to 20,000 Hz). The speaker 1205 can include a driver to produce acoustic waves with frequencies outside the audible range, such as 0 to 60 Hz, or in the ultrasonic range such as 20 kHz to 4 GHz. The speaker 1205 can include one or more transducers or drivers to produce sounds at varying portions of the audible frequency range. For example, the speaker 1205 can include tweeters for high range frequencies (e.g., 2,000 Hz to 20,000 Hz), mid-range drivers for middle frequencies (e.g., 250 Hz to 2000 Hz), or woofers for low frequencies (e.g., 60 Hz to 250 Hz).

The speaker 1205 can include one or more types of speaker hardware, components or technology to produce sound. For example, the speaker 1205 can include a diaphragm to produce sound. The speaker 1205 can include a moving-iron loudspeaker that uses a stationary coil to vibrate a magnetized piece of metal. The speaker 1205 can include a piezoelectric speaker. A piezoelectric speaker can use the piezoelectric effect to generate sound by applying a voltage to a piezoelectric material to generate motion, which is converted into audible sound using diaphragms and resonators.

The speaker 1205 can include various other types of hardware or technology, such as magnetostatic loudspeakers, magnetostrictive speakers, electrostatic loudspeakers, a ribbon speaker, planar magnetic loudspeakers, bending wave loudspeakers, coaxial drivers, horn loudspeakers, Heil air motion transducers, or transparent ionic conductions speaker.

In some cases, the speaker 1205 may not include a diaphragm. For example, the speaker 1205 can be a plasma arc speaker that uses electrical plasma as a radiating element. The speaker 1205 can be a thermoacoustic speakers that uses carbon nanotube thin film. The speaker 1205 can be a rotary woofer that includes a fan with blades that constantly change their pitch.

In some embodiments, the speaker 1205 can include a headphone or a pair of headphones, earspeakers, earphones, or earbuds. Headphones can be relatively small speakers as compared to loudspeakers. Headphones can be designed and constructed to be placed in the ear, around the ear, or otherwise at or near the ear. Headphones can include electroacoustic transducers that convert an electrical signal to a corresponding sound in the subject's ear. In some embodiments, the headphones 1205 can include or interface with a headphone amplifier, such as an integrated amplifier or a standalone unit.

In some embodiments, the speaker 1205 can include headphones that can include an air jet that pushes air into the auditory canal, pushing the tympanum in a manner similar to that of a sound wave. The compression and rarefaction of the tympanic membrane through bursts of air (with or without any discernible sound) can control frequencies of neural oscillations similar to auditory signals. For example, the speaker 1205 can include air jets or a device that resembles in-ear headphones that either push, pull or both push and pull air into and out of the ear canal in order to compress or pull the tympanic membrane to affect the frequencies of neural oscillations. The NSS 905 can instruct, configure or cause the air jets to generate bursts of air at a predetermined frequency.

In some embodiments, the headphones can connect to the audio signaling component 950 via a wired or wireless connection. In some embodiments, the audio signaling component 950 can include the headphones. In some embodiments, the headphones 1205 can interface with one or more components of the NSS 905 via a wired or wireless connection. In some embodiments, the headphones 1205 can include one or more components of the NSS 905 or system 100, such as the audio generation module 910, audio adjustment module 915, unwanted frequency filtering module 920, profile manager 925, side effects management module 930, feedback monitor 935, audio signaling component 950, filtering component 955, or feedback component 960.

The speaker 1205 can include or be integrated into various types of headphones. For example, the headphones can include, for example, circumaural headphones (e.g., full size headphones) that include circular or ellipsoid earpads that are designed and constructed to seal against the head to attenuate external noise. Circumaural headphones can facilitate providing an immersive auditory brainwave wave stimulation experience, while reducing external distractions. In some embodiments, headphones can include supra-aural headphones, which include pads that press against the ears rather than around them. Supra-aural headphones may provide less attenuation of external noise.

Both circumaural headphones and supra-aural headphones can have an open back, closed back, or semi open back. An open back leaks more sound and allows more ambient sounds to enter, but provides a more natural or speaker-like sound. Closed back headphones block more of the ambient noise as compared to open back headphones, thus providing a more immersive auditory brainwave stimulation experience while reducing external distractions.

In some embodiments, headphones can include ear-fitting headphones, such as earphones or in-ear headphones. Earphones (or earbuds) can refer to small headphones that are fitted directly in the outer ear, facing but not inserted in the ear canal. Earphones, however, provide minimal acoustic isolation and allow ambient noise to enter. In-ear headphones (or in-ear monitors or canalphones) can refer to small headphones that can be designed and constructed for insertion into the ear canal. In-ear headphones engage the ear canal and can block out more ambient noise as compared to earphones, thus providing a more immersive auditory brainwave stimulation experience. In-ear headphones can include ear canal plugs made or formed from one or more material, such as silicone rubber, elastomer, or foam. In some embodiments, in-ear headphones can include custom-made castings of the ear canal to create custom-molded plugs that provide added comfort and noise isolation to the subject, thereby further improving the immersiveness of the auditory brainwave stimulation experience.

In some embodiments, one or more microphones 1210 can be used to detect sound. A microphone 1210 can be integrated with a speaker 1205. The microphone 1210 can provide feedback information to the NSS 905 or other component of system 100. The microphone 1210 can provide feedback to a component of the speaker 1205 to cause the speaker 1205 to adjust a parameter of audio signal.

The microphone 1210 can include a transducer that converts sound into an electrical signal. The Microphone 1210 can use electromagnetic induction, capacitance change, or piezoelectricity to produce the electrical signal from air pressure variations. In some cases, the microphone 1210 can include or be connected to a pre-amplifier to amplify the signal before it is recorded or processed. The microphone 1210 can include one or more type of microphone, including, for example, a condenser microphone, RF condenser microphone, electret condenser, dynamic microphone, moving-coil microphone, ribbon microphone, carbon microphone, piezoelectric microphone, crystal microphone, fiber optic microphone, laser microphone, liquid or water microphone, microelectromechanical systems (“MEMS”) microphone, or speakers as microphones.

The feedback component 960 can include or interface with the microphone 1210 to obtain, identify, or receive sound. The feedback component 960 can obtain ambient noise. The feedback component 960 can obtain sound from the speakers 1205 to facilitate the NSS 905 adjusting a characteristic of the audio signal generated by the speaker 1205. The microphone 1210 can receive voice input from the subject, such as audio commands, instructions, requests, feedback information, or responses to survey questions.

In some embodiments, one or more speakers 1205 can be integrated with one or more microphones 1210. For example, the speaker 1205 and microphone 1210 can form a headset, be placed in a single enclosure, or may even be the same device since the speaker 1205 and the microphone 1210 may be structurally designed to toggle between a sound generation mode and a sound reception mode.

FIG. 12B illustrates a system configuration for auditory brain entrainment in accordance with an embodiment. The system 1200 can include at least one speaker 1205. The system 1200 can include at least microphone 1210. The system 1200 can include at least one active noise cancellation component 1215. The system 1200 can include at least one feedback sensor 1225. The system 1200 can include or interface with the NSS 905. The system 1200 can include or interface with an audio player 1220.

The system 1200 can include a first speaker 1205 positioned at a first ear. The system 1200 can include a second speaker 1205 positioned at a second year. The system 1200 can include a first active noise cancellation component 1215 communicatively coupled with the first microphone 1210. The system 1200 can include a second active noise cancellation component 1215 communicatively coupled with the second microphone 1210. In some cases, the active noise cancellation component 1215 can communicate with both the first speaker 1205 and the second speaker 1205, or both the first microphone 1210 and the second microphone 1210. The system 1200 can include a first microphone 1210 communicatively coupled with the active noise cancellation component 1215. The system 1200 can include a second microphone 1210 communicatively coupled with the active noise cancelation component 1215. In some embodiments, each of the microphone 1210, speaker 1205 and active noise cancellation component can communicate or interface with the NSS 905. In some embodiments, the system 1200 can include a feedback sensor 1225 and a second feedback sensor 1225 communicatively coupled to the NSS 905, the speaker 1205, microphone 1210, or active noise cancellation component 1215.

In operation, and in some embodiments, the audio player 1220 can play a musical track. The audio player 1220 can provide the audio signal corresponding to the musical track via a wired or wireless connection to the first and second speakers 1205. In some embodiments, the NSS 905 can intercept the audio signal from the audio player. For example, the NSS 905 can receive the digital or analog audio signal from the audio player 1220. The NSS 905 can be intermediary to the audio player 1220 and a speaker 1205. The NSS 905 can analyze the audio signal corresponding to the music in order to embed an auditory brainwave stimulation signal. For example, the NSS 905 can adjust the volume of the auditory signal from the audio player 1220 to generate acoustic pulses having a pulse rate interval as depicted in FIG. 11C. In some embodiments, the NSS 905 can use a binaural beats technique to provide different auditory signals to the first and second speakers that, when perceived by the brain, is combined to have the desired stimulation frequency.

In some embodiments, the NSS 905 can adjust for any latency between first and second speakers 1205 such that the brain perceives the audio signals at the same or substantially same time (e.g., within 1 milliseconds, 2 milliseconds, 5 milliseconds, or 10 milliseconds). The NSS 905 can buffer the audio signals to account for latency such that audio signals are transmitted from the speakers at the same time.

In some embodiments, the NSS 905 may not be intermediary to the audio player 1220 and the speaker. For example, the NSS 905 can receive the musical track from a digital music repository. The NSS 905 can manipulate or modify the musical track to embed acoustic pulses in accordance with the desired PRI. The NSS 905 can then provide the modified musical track to the audio player 1220 to provide the modified audio signal to the speaker 1205.

In some embodiments, an active noise cancellation component 1215 can receive ambient noise information from the microphone 1210, identify unwanted frequencies or noise, and generate an inverted phase waveform to cancel out or attenuate the unwanted waveforms. In some embodiments, the system 1200 can include an additional speaker that generates the noise canceling waveform provided by the noise cancellation component 1215. The noise cancellation component 1215 can include the additional speaker.

The feedback sensor 1225 of the system 1200 can detect feedback information, such as environmental parameters or physiological conditions. The feedback sensor 1225 can provide the feedback information to NSS 905. The NSS 905 can adjust or change the audio signal based on the feedback information. For example, the NSS 905 can determine that a pulse rate of the subject exceeds a predetermined threshold, and then lower the volume of the audio signal. The NSS 905 can detect that the volume of the auditory signal exceeds a threshold, and decrease the amplitude. The NSS 905 can determine that the pulse rate interval is below a threshold, which can indicate that a subject is losing focus or not paying a satisfactory level of attention to the audio signal, and the NSS 905 can increase the amplitude of the audio signal or change the tone or music track. In some embodiments, the NSS 905 can vary the tone or the music track based on a time interval. Varying the tone or the music track can cause the subject to pay a greater level of attention to the auditory stimulation, which can facilitate brainwave entrainment.

In some embodiments, the NSS 905 can receive neural oscillation information from EEG probes 1225, and adjust the auditory stimulation based on the EEG information. For example, the NSS 905 can determine, from the probe information, that neurons are oscillating at an undesired frequency. The NSS 905 can then identify the corresponding undesired frequency in ambient noise using the microphone 1210. The NSS 905 can then instruct the active noise cancellation component 1215 to cancel out the waveforms corresponding to the ambient noise having the undesired frequency.

In some embodiments, the NSS 905 can enable a passive noise filter. A pass noise filter can include a circuit having one or more or a resistor, capacitor or an inductor that filters out undesired frequencies of noise. In some cases, a passive filter can include a sound insulating material, sound proofing material, or sound absorbing material.

FIG. 4C illustrates a system configuration for auditory brain entrainment in accordance with an embodiment. The system 401 can provide auditory brainwave stimulation using ambient noise source 1230. For example, system 401 can include the microphone 1210 that detects the ambient noise 1230. The microphone 1210 can provide the detected ambient noise to NSS 905. The NSS 905 can modify the ambient noise 1230 before providing it to the first speaker 1205 or the second speaker 1205. In some embodiments, the system 401 can be integrated or interface with a hearing aid device. A hearing aid can be a device designed to improve hearing.

The NSS 905 can increase or decrease the amplitude of the ambient noise 1230 to generate acoustic bursts having the desired pulse rate interval. The NSS 905 can provide the modified audio signals to the first and second speakers 1205 to facilitate auditory brainwave entrainment.

In some embodiments, the NSS 905 can overlay a click train, tones, or other acoustic pulses over the ambient noise 1230. For example, the NSS 905 can receive the ambient noise information from the microphone 1210, apply an auditory stimulation signal to the ambient noise information, and then present the combined ambient noise information and auditory stimulation signal to the first and second speakers 1205. In some cases, the NSS 905 can filter out unwanted frequencies in the ambient noise 1230 prior to providing the auditory stimulation signal to the speakers 1205.

Thus, using the ambient noise 1230 as part of the auditory stimulation, a subject can observe the surroundings or carry on with their daily activities while receiving auditory stimulation to facilitate brainwave entrainment.

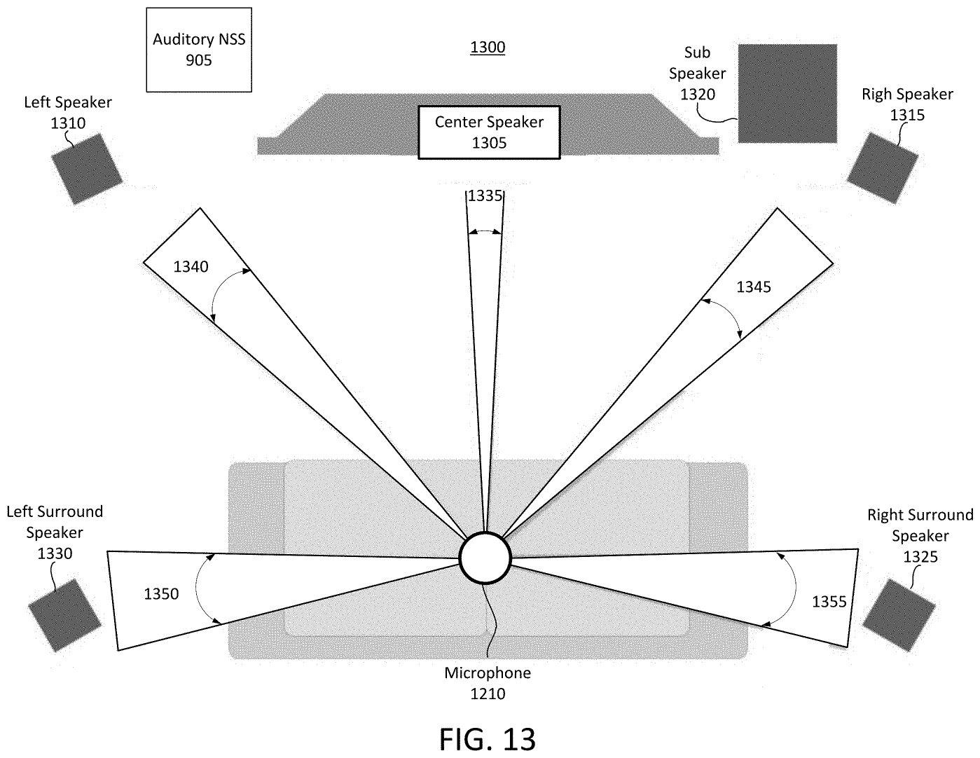

FIG. 13 illustrates a system configuration for auditory brain entrainment in accordance with an embodiment. The system 1300 can provide auditory stimulation for brainwave entrainment using a room environment. The system 1300 can include one or more speakers. The system 1300 can include a surround sound system. For example, the system 1300 includes a left speaker 1310, right speaker 1315, center speaker 1305, right surround speaker 1325, and left surround speaker 1330. System 1300 an include a sub-woofer 1320. The system 1300 can include the microphone 1210. The system 1300 can include or refer to a 5.1 surround system. In some embodiments, the system 1300 can have 1, 2, 3, 4, 5, 6, 7 or more speakers.

When providing auditory stimulation using a surround system, the NSS 905 can provide the same or different audio signals to each of the speakers in the system 1300. The NSS 905 can modify or adjust audio signals provided to one or more of the speakers in system 1300 in order to facilitate brainwave entrainment. For example, the NSS 905 can receive feedback from microphone 1210 and modify, manipulate or otherwise adjust the audio signal to optimize the auditory stimulation provided to a subject located at a position in the room that corresponds to the location of the microphone 1210. The NSS 905 can optimize or improve the auditory stimulation perceived at the location corresponding to microphone 1210 by analyzing the acoustic beams or waves generated by the speakers that propagate towards the microphone 1210.

The NSS 905 can be configured with information about the design and construction of each speaker. For example, speaker 1305 can generate sound in a direction that has an angle of 1335; speaker 1310 can generate sound that travels in a direction having an angle of 1340; speaker 1315 can generate sound that travels in a direction having an angle of 1345; speaker 1325 can generate sound that travels in a direction having an angle of 1355; and speaker 1330 can generate sound that travels in a direction having an angle of 1350. These angles can be the optimal or predetermined angles for each of the speakers. These angles can refer to the optimal angle of each speaker such that a person positioned at location corresponding to microphone 1210 can receive the optimum auditory stimulation. Thus, the speakers in system 1300 can be oriented to transmit auditory stimulation towards the subject.

In some embodiments, the NSS 905 can enable or disable one or more speakers. In some embodiments, the NSS 905 can increase or decrease the volume of the speakers to facilitate brainwave entrainment. The NSS 905 can intercept musical tracks, television audio, movie audio, interne audio, audio output from a set top box, or other audio source. The NSS 905 can adjust or manipulate the received audio, and transmit the adjusted audio signals to the speakers in system 1300 to induce brainwave entrainment.

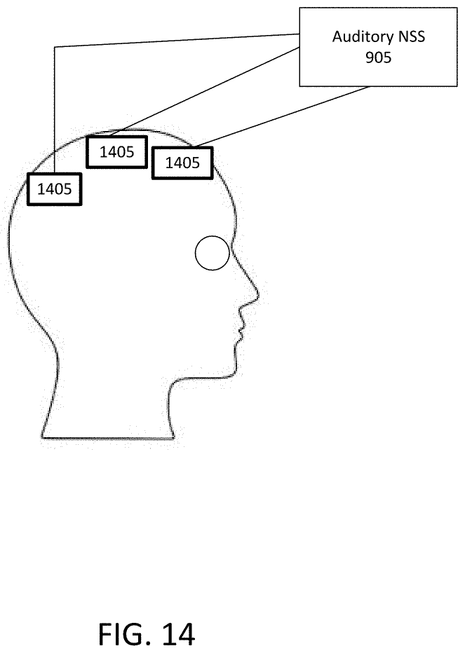

FIG. 14 illustrates feedback sensors 1405 placed or positioned at, on, or near a person's head. Feedback sensors 1405 can include, for example, EEG probes that detect brain wave activity.

The feedback monitor 935 can detect, receive, obtain, or otherwise identify feedback information from the one or more feedback sensors 1405. The feedback monitor 935 can provide the feedback information to one or more component of the NSS 905 for further processing or storage. For example, the profile manager 925 can update profile data structure 945 stored in data repository 940 with the feedback information. Profile manager 925 can associate the feedback information with an identifier of the patient or person undergoing the auditory brain stimulation, as well as a time stamp and date stamp corresponding to receipt or detection of the feedback information.

The feedback monitor 935 can determine a level of attention. The level of attention can refer to the focus provided to the acoustic pulses used for brain stimulation. The feedback monitor 935 can determine the level of attention using various hardware and software techniques. The feedback monitor 935 can assign a score to the level of attention (e.g., 1 to 10 with 1 being low attention and 10 being high attention, or vice versa, 1 to 100 with 1 being low attention and 100 being high attention, or vice versa, 0 to 1 with 0 being low attention and 1 being high attention, or vice versa), categorize the level of attention (e.g., low, medium, high), grade the attention (e.g., A, B, C, D, or F), or otherwise provide an indication of a level of attention.

In some cases, the feedback monitor 935 can track a person's eye movement to identify a level of attention. The feedback monitor 935 can interface with a feedback component 960 that includes an eye-tracker. The feedback monitor 935 (e.g., via feedback component 960) can detect and record eye movement of the person and analyze the recorded eye movement to determine an attention span or level of attention. The feedback monitor 935 can measure eye gaze which can indicate or provide information related to covert attention. For example, the feedback monitor 935 (e.g., via feedback component 960) can be configured with electro-oculography (“EOG”) to measure the skin electric potential around the eye, which can indicate a direction the eye faces relative to the head. In some embodiments, the EOG can include a system or device to stabilize the head so it cannot move in order to determine the direction of the eye relative to the head. In some embodiments, the EOG can include or interface with a head tracker system to determine the position of the heads, and then determine the direction of the eye relative to the head.

In some embodiments, the feedback monitor 935 and feedback component 960 can determine a level of attention the subject is paying to the auditory stimulation based on eye movement. For example, increased eye movement may indicate that the subject is focusing on visual stimuli, as opposed to the auditory stimulation. To determine the level of attention the subject is paying to visual stimuli as opposed to the auditory stimulation, the feedback monitor 935 and feedback component 960 can determine or track the direction of the eye or eye movement using video detection of the pupil or corneal reflection. For example, the feedback component 960 can include one or more camera or video camera. The feedback component 960 can include an infra-red source that sends light pulses towards the eyes. The light can be reflected by the eye. The feedback component 960 can detect the position of the reflection. The feedback component 960 can capture or record the position of the reflection. The feedback component 960 can perform image processing on the reflection to determine or compute the direction of the eye or gaze direction of the eye.

The feedback monitor 935 can compare the eye direction or movement to historical eye direction or movement of the same person, nominal eye movement, or other historical eye movement information to determine a level of attention. For example, the feedback monitor 935 can determine a historical amount of eye movement during historical auditory stimulation sessions. The feedback monitor 935 can compare the current eye movement with the historical eye movement to identify a deviation. The NSS 905 can determine, based on the comparison, an increase in eye movement and further determine that the subject is paying less attention to the current auditory stimulation based on the increase in eye movement. In response to detecting the decrease in attention, the feedback monitor 935 can instruct the audio adjustment module 915 to change a parameter of the audio signal to capture the subject's attention. The audio adjustment module 915 can change the volume, tone, pitch, or music track to capture the subject's attention or increase the level of attention the subject is paying to the auditory stimulation. Upon changing the audio signal, the NSS 905 can continue to monitor the level of attention. For example, upon changing the audio signal, the NSS 905 can detect a decrease in eye movement which can indicate an increase in a level of attention provided to the audio signal.

The feedback sensor 1405 can interact with or communicate with NSS 905. For example, the feedback sensor 1405 can provide detected feedback information or data to the NSS 905 (e.g., feedback monitor 935). The feedback sensor 1405 can provide data to the NSS 905 in real-time, for example as the feedback sensor 1405 detects or senses or information. The feedback sensor 1405 can provide the feedback information to the NSS 905 based on a time interval, such as 1 minute, 2 minutes, 5 minutes, 10 minutes, hourly, 2 hours, 4 hours, 12 hours, or 24 hours. The feedback sensor 1405 can provide the feedback information to the NSS 905 responsive to a condition or event, such as a feedback measurement exceeding a threshold or falling below a threshold. The feedback sensor 1405 can provide feedback information responsive to a change in a feedback parameter. In some embodiments, the NSS 905 can ping, query, or send a request to the feedback sensor 1405 for information, and the feedback sensor 1405 can provide the feedback information in response to the ping, request, or query.

### Method for Neural Stimulation via Auditory Stimulation

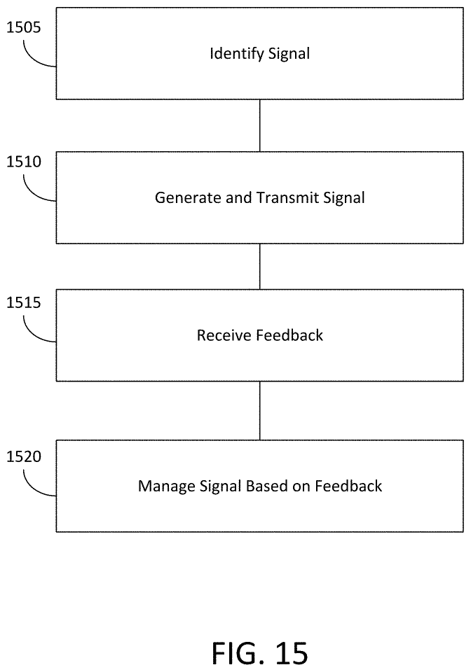

FIG. 15 is a flow diagram of a method of performing auditory brain entrainment in accordance with an embodiment. The method 800 can be performed by one or more system, component, module or element depicted in FIGS. 7A, 7B, and 9-14, including, for example, a neural stimulation system (NSS). In brief overview, the NSS can identify an audio signal to provide at block 1505. At block 1510, the NSS can generate and transmit the identified audio signal. At 1515 the NSS can receive or determine feedback associated with neural activity, physiological activity, environmental parameters, or device parameters. At 1520 the NSS can manage, control, or adjust the audio signal based on the feedback.

NSS Operating with Headphones

The NSS 905 can operate in conjunction with the speakers 1205 as depicted in FIG. 12A. The NSS 905 can operate in conjunction with earphones or in-ear phones including the speaker 1205 and a feedback sensor 1405.

In operation, a subject using the headphones can wear the headphones on their head such that speakers or placed at or in the ear canals. In some cases, the subject can provide an indication to the NSS 905 that the headphones have been worn and that the subject is ready to undergo brainwave entrainment. The indication can include an instruction, command, selection, input, or other indication via an input/output interface, such as a keyboard 726, pointing device 727, or other I/O devices 730a-n. The indication can be a motion-based indication, visual indication, or voice-based indication. For example, the subject can provide a voice command that indicates that the subject is ready to undergo brainwave entrainment.

In some cases, the feedback sensor 1405 can determine that the subject is ready to undergo brainwave entrainment. The feedback sensor 1405 can detect that the headphones have been placed on a subject's head. The NSS 905 can receive motion data, acceleration data, gyroscope data, temperature data, or capacitive touch data to determine that the headphones have been placed on the subject's head. The received data, such as motion data, can indicate that the headphones were picked up and placed on the subject's head. The temperature data can measure the temperature of or proximate to the headphones, which can indicate that the headphones are on the subject's head. The NSS 905 can detect that the subject is ready responsive to determining that the subject is paying a high level of attention to the headphones or feedback sensor 1405.

Thus, the NSS 905 can detect or determine that the headphones have been worn and that the subject is in a ready state, or the NSS 905 can receive an indication or confirmation from the subject that the subject has worn the headphones and the subject is ready to undergo brainwave entrainment. Upon determining that the subject is ready, the NSS 905 can initialize the brainwave entrainment process. In some embodiments, the NSS 905 can access a profile data structure 945. For example, a profile manager 925 can query the profile data structure 945 to determine one or more parameter for the external auditory stimulation used for the brain entrainment process. Parameters can include, for example, a type of audio stimulation technique, an intensity or volume of the audio stimulation, frequency of the audio stimulation, duration of the audio stimulation, or wavelength of the audio stimulation. The profile manager 925 can query the profile data structure 945 to obtain historical brain entrainment information, such as prior auditory stimulation sessions. The profile manager 925 can perform a lookup in the profile data structure 945. The profile manager 925 can perform a look-up with a username, user identifier, location information, fingerprint, biometric identifier, retina scan, voice recognition and authentication, or other identifying technique.

The NSS 905 can determine a type of external auditory stimulation based on the components connected to the headphones. The NSS 905 can determine the type of external auditory stimulation based on the type of speakers 1205 available. For example, if the headphones are connected to an audio player, the NSS 905 can determined to embed acoustic pulses. If the headphones are not connected to an audio player, but only the microphone, the NSS 905 can determine to inject a pure tone or modify ambient noise.

In some embodiments, the NSS 905 can determine the type of external auditory stimulation based on historical brainwave entrainment sessions. For example, the profile data structure 945 can be pre-configured with information about the type of audio signaling component 950.

The NSS 905 can determine, via the profile manager 925, a modulation frequency for the pulse train or the audio signal. For example, NSS 905 can determine, from the profile data structure 945, that the modulation frequency for the external auditory stimulation should be set to 40 Hz. Depending on the type of auditory stimulation, the profile data structure 945 can further indicate a pulse length, intensity, wavelength of the acoustic wave forming the audio signal, or duration of the pulse train.

In some cases, the NSS 905 can determine or adjust one or more parameter of the external auditory stimulation. For example, the NSS 905 (e.g., via feedback component 960 or feedback sensor 1405) can determine an amplitude of the acoustic wave or volume level for the sound. The NSS 905 (e.g., via audio adjustment module 915 or side effects management module 930) can establish, initialize, set, or adjust the amplitude or wavelength of the acoustic waves or acoustic pulses. For example, the NSS 905 can determine that there is a low level of ambient noise. Due to the low level of ambient noise, subject's hearing may not be impaired or distracted. The NSS 905 can determine, based on detecting a low level of ambient noise, that it may not be necessary to increase the volume, or that it may be possible to reduce the volume to maintain the efficacy of brainwave entrainment.

In some embodiments, the NSS 905 can monitor (e.g., via feedback monitor 935 and feedback component 960) the level of ambient noise throughout the brainwave entrainment process to automatically and periodically adjust the amplitude of the acoustic pulses. For example, if the subject began the brainwave entrainment process when there was a high level of ambient noise, the NSS 905 can initially set a higher amplitude for the acoustic pulses and use a tone that includes frequencies that are easier to perceive, such as 10 kHz. However, in some embodiments in which the ambient noise level decreases throughout the brainwave entrainment process, the NSS 905 can automatically detect the decrease in ambient noise and, in response to the detection, adjust or lower the volume while decreasing the frequency of the acoustic wave. The NSS 905 can adjust the acoustic pulses to provide a high contrast ratio with respect to ambient noise to facilitate brainwave entrainment.

In some embodiments, the NSS 905 (e.g., via feedback monitor 935 and feedback component 960) can monitor or measure physiological conditions to set or adjust a parameter of the acoustic wave. In some embodiments, the NSS 905 can monitor or measure heart rate, pulse rate, blood pressure, body temperature, perspiration, or brain activity to set or adjust a parameter of the acoustic wave.

In some embodiments, the NSS 905 can be preconfigured to initially transmit acoustic pulses having a lowest setting for the acoustic wave intensity (e.g., low amplitude or high wavelength) and gradually increase the intensity (e.g., increase the amplitude of the or decrease the wavelength) while monitoring feedback until an optimal audio intensity is reached. An optimal audio intensity can refer to a highest intensity without adverse physiological side effects, such as deafness, seizures, heart attack, migraines, or other discomfort. The NSS 905 (e.g., via side effects management module 930) can monitor the physiological symptoms to identify the adverse side effects of the external auditory stimulation, and adjust (e.g., via audio adjustment module 915) the external auditory stimulation accordingly to reduce or eliminate the adverse side effects.

In some embodiments, the NSS 905 (e.g., via audio adjustment module 915) can adjust a parameter of the audio wave or acoustic pulse based on a level of attention. For example, during the brainwave entrainment process, the subject may get bored, lose focus, fall asleep, or otherwise not pay attention to the acoustic pulses. Not paying attention to the acoustic pulses may reduce the efficacy of the brainwave entrainment process, resulting in neurons oscillating at a frequency different from the desired modulation frequency of the acoustic pulses.

NSS 905 can detect the level of attention the subject is paying to the acoustic pulses using the feedback monitor 935 and one or more feedback component 960. Responsive to determining that the subject is not paying a satisfactory amount of attention to the acoustic pulses, the audio adjustment module 915 can change a parameter of the audio signal to gain the subject's attention. For example, the audio adjustment module 915 can increase the amplitude of the acoustic pulse, adjust the tone of the acoustic pulse, or change the duration of the acoustic pulse. The audio adjustment module 915 can randomly vary one or more parameters of the acoustic pulse. The audio adjustment module 915 can initiate an attention seeking acoustic sequence configured to regain the subject's attention. For example, the audio sequence can include a change in frequency, tone, amplitude, or insert words or music in a predetermined, random, or pseudo-random pattern. The attention seeking audio sequence can enable or disable different acoustic sources if the audio signaling component 950 includes multiple audio sources or speakers. Thus, the audio adjustment module 915 can interact with the feedback monitor 935 to determine a level of attention the subject is providing to the acoustic pulses, and adjust the acoustic pulses to regain the subject's attention if the level of attention falls below a threshold.

In some embodiments, the audio adjustment module 915 can change or adjust one or more parameter of the acoustic pulse or acoustic wave at predetermined time intervals (e.g., every 5 minutes, 10 minutes, 15 minutes, or 20 minutes) to regain or maintain the subject's attention level.

In some embodiments, the NSS 905 (e.g., via unwanted frequency filtering module 920) can filter, block, attenuate, or remove unwanted auditory external stimulation. Unwanted auditory external stimulation can include, for example, unwanted modulation frequencies, unwanted intensities, or unwanted wavelengths of sound waves. The NSS 905 can deem a modulation frequency to be unwanted if the modulation frequency of a pulse train is different or substantially different (e.g., 1%, 2%, 5%, 10%, 15%, 20%, 25%, or more than 25%) from a desired frequency.

For example, the desired modulation frequency for brainwave entrainment can be 40 Hz. However, a modulation frequency of 20 Hz or 80 Hz can reduce the beneficial effects to cognitive functioning of the brain, a cognitive state of the brain, the immune system, or inflammation that can result from brainwave entrainment at other frequencies, such as 40 Hz. Thus, the NSS 905 can filter out the acoustic pulses corresponding to the 20 Hz or 80 Hz modulation frequency.

In some embodiments, the NSS 905 can detect, via feedback component 960, that there are acoustic pulses from an ambient noise source that corresponds to an unwanted modulation frequency of 20 Hz. The NSS 905 can further determine the wavelength of the acoustic waves of the acoustic pulses corresponding to the unwanted modulation frequency. The NSS 905 can instruct the filtering component 955 to filter out the wavelength corresponding to the unwanted modulation frequency.

### Neural Stimulation Via Multiple Modes of Stimulation

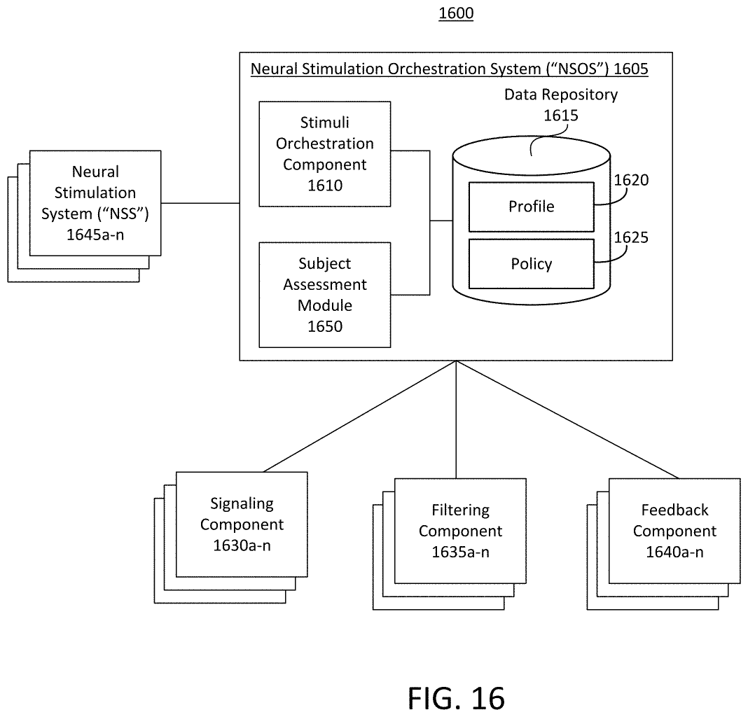

FIG. 16 is a block diagram depicting a system for neural stimulation via multiple modes of stimulation in accordance with an embodiment. The system 1600 can include a neural stimulation orchestration system (“NS OS”) 1605. The NSOS 1605 can provide multiple modes of stimulation. For example, the NSOS 1605 can provide a first mode of stimulation that includes visual stimulation, and a second mode of stimulation that includes auditory stimulation. For each mode of stimulation, the NSOS 1605 can provide a type of signal. For example, for the visual mode of stimulation, the NSOS 1605 can provide the following types of signals: light pulses, image patterns, flicker of ambient light, or augmented reality. NSOS 1605 can orchestrate, manage, control, or otherwise facilitate providing multiple modes of stimulation and types of stimulation.

In brief overview, the NSOS 1605 can include, access, interface with, or otherwise communicate with one or more of a stimuli orchestration component 1610, a subject assessment module 1650, a data repository 1615, one or more signaling components 1630a-n, one or more filtering components 1635a-n, one or more feedback components 1640a-n, and one or more neural stimulation systems (“NSS”) 1645a-n. The data repository 1615 can include or store a profile data structure 1620 and a policy data structure 1625. The stimuli orchestration component 1610 and subject assessment module 1650 can include at least one processing unit or other logic device such as programmable logic array engine, or module configured to communicate with the database repository 1615. The stimuli orchestration component 1610 and subject assessment module 1650 can be a single component, include separate components, or be part of the NSOS 1605. The system 1600 and its components, such as the NSOS 1605, may include hardware elements, such as one or more processors, logic devices, or circuits. The system 1600 and its components, such as the NSOS 1605, can include one or more hardware or interface component depicted in system 700 in FIGS. 7A and 7B. For example, a component of system 1600 can include or execute on one or more processors 721, access storage 728 or memory 722, and communicate via network interface 718. The system 1600 can include one or more component or functionality depicted in FIGS. 1-15, including, for example, system 100, system 900, visual NSS 105, or auditory NSS 905. For example, at least one of the signaling components 1630a-n can include one or more component or functionality of visual signaling component 150 or audio signaling component 950. At least one of the filtering components 1635a-n can include one or more component or functionality of filtering component 155 or filtering component 955. At least one of the feedback components 1640a-n can include one or more component or functionality of feedback component 160 or feedback component 960. At least one of the NSS 1645a-n can include one or more component or functionality of visual NSS 105 or auditory NSS 905.

Still referring to FIG. 16, and in further detail, the NSOS 1605 can include at least stimuli orchestration component 1610. The stimuli orchestration component 1610 can be designed and constructed to perform neural stimulation using multiple modalities of stimulation. The stimuli orchestration component 1610 or NSOS 1605 can interface with at least one of the signaling components 1630a-n, at least one of the filtering components 1635a-n or at least one of the feedback components 1640a-n. One or more of the signaling components 1630a-n can be a same type of signaling component or a different type of signaling component. The type of signaling component can correspond to a mode of stimulation. For example, multiple types of signaling components 1630a-n can correspond to visual signaling components or auditory signaling components. In some cases, at least one of the signaling components 1630a-n includes a visual signaling component 150 such as a light source, LED, laser, tablet computing device, or virtual reality headset. At least one of the signaling components includes an audio signaling component 950, such as headphones, speakers, cochlear implants, or air jets.

One or more of the filtering components 1635a-n can be a same type of filtering component or a different type of filtering component. One or more of the feedback components 1640a-n can be a same type of feedback component or a different type of feedback component. For example, the feedback components 1640a-n can include an electrode, dry electrode, gel electrode, saline soaked electrode, adhesive-based electrodes, a temperature sensor, heart or pulse rate monitor, physiological sensor, ambient light sensor, ambient temperature sensor, sleep status via actigraphy, blood pressure monitor, respiratory rate monitor, brain wave sensor, EEG probe, EOG probes configured measure the corneo-retinal standing potential that exists between the front and the back of the human eye, accelerometer, gyroscope, motion detector, proximity sensor, camera, microphone, or photo detector.

The stimuli orchestration component 1610 can include or be configured with an interface to communicate with different types of signaling components 1630a-n, filtering components 1635a-n or feedback components 1640a-n. The NSOS 1605 or stimuli orchestration component 1610 can interface with system intermediary to one of the signaling components 1630a-n, filtering components 1635a-n, or feedback components 1640a-n. For example, the stimuli orchestration component 1610 can interface with the visual NSS 105 depicted in FIG. 1 or auditory NSS 905 depicted in FIG. 9. Thus, in some embodiments, the stimuli orchestration component 1610 or NSOS 1605 can indirectly interface with at least one of the signaling components 1630a-n, filtering components 1635a-n, or feedback components 1640a-n.

The stimuli orchestration component 1610 (e.g., via the interface) can ping each of the signaling components 1630a-n, filtering components 1635a-n, and feedback components 1640a-n to determine information about the components. The information can include a type of the component (e.g., visual, auditory, attenuator, optical filter, temperature sensor, or light sensor), configuration of the component (e.g., frequency range, amplitude range), or status information (e.g., standby, ready, online, enabled, error, fault, offline, disabled, warning, service needed, availability, or battery level).

The stimuli orchestration component 1610 can instruct or cause at least one of the signaling components 1630a-n to generate, transmit or otherwise provide a signal that can be perceived, received or observed by the brain and affect a frequency of neural oscillations in at least one region or portion of a subject's brain. The signal can be perceived via various means, including, for example, optical nerves or cochlear cells.

The stimuli orchestration component 1610 can access the data repository 1615 to retrieve profile information 1620 and a policy 1625. The profile information 1620 can include profile information 145 or profile information 945. The policy 1625 can include a multi-modal stimulation policy. The policy 1625 can indicate a multi-modal stimulation program. The stimuli orchestration component 1610 can apply the policy 1625 to profile information to determine a type of stimulation (e.g., visual or auditory) and determine a value for a parameter for each type of stimulation (e.g., amplitude, frequency, wavelength, color, etc.). The stimuli orchestration component 1610 can apply the policy 1625 to the profile information 1620 and feedback information received from one or more feedback components 1640a-n to determine or adjust the type of stimulation (e.g., visual or auditory) and determine or adjust the value parameter for each type of stimulation (e.g., amplitude, frequency, wavelength, color, etc.). The stimuli orchestration component 1610 can apply the policy 1625 to profile information to determine a type of filter to be applied by at least one of the filtering components 1635a-n (e.g., audio filter or visual filter) and determine a value for a parameter for the type of filter (e.g., frequency, wavelength, color, sound attenuation, etc.). The stimuli orchestration component 1610 can apply the policy 1625 to profile information and feedback information received from one or more feedback components 1640a-n to determine or adjust the type of filter to be applied by at least one of the filtering components 1635a-n (e.g., audio filter or visual filter) and determine or adjust the value for the parameter for filter (e.g., frequency, wavelength, color, sound attenuation, etc.).

The NSOS 1605 can obtain the profile information 1620 via a subject assessment module 1650. The subject assessment module 1650 can be designed and constructed to determine, for one or more subjects, information that can facilitate neural stimulation via one or more modes of stimulation. The subject assessment module 1650 can receive, obtain, detect, determine or otherwise identify the information via feedback components 1640a-n, surveys, queries, questionnaires, prompts, remote profile information accessible via a network, diagnostic tests, or historical treatments.

The subject assessment module 1650 can receive the information prior to initiating neural stimulation, during neural stimulation, or after neural stimulation. For example, the subject assessment module 1650 can provide a prompt with a request for information prior to initiating the neural stimulation session. The subject assessment module 1650 can provide a prompt with a request for information during the neural stimulation session. The subject assessment module 1650 can receive feedback from feedback component 1640a-n (e.g., an EEG probe) during the neural stimulation session. The subject assessment module 1650 can provide a prompt with a request for information subsequent to termination of the neural stimulation session. The subject assessment module 1650 can receive feedback from feedback component 1640a-n subsequent to termination of the neural stimulation session.

The subject assessment module 1650 can use the information to determine an effectiveness of a modality of stimulation (e.g., visual stimulation or auditory stimulation) or a type of signal (e.g., light pulse from a laser or LED source, ambient light flicker, or image pattern displayed by a tablet computing device). For example, the subject assessment module 1650 can determine that the desired neural stimulation resulted from a first mode of stimulation or first type of signal, while the desired neural stimulation did not occur or took longer to occur with the second mode of stimulation or second type of signal. The subject assessment module 1650 can determine that the desired neural stimulation was less pronounced from the second mode of stimulation or second type of signal relative to the first mode of stimulation or first type of signal based on feedback information from a feedback component 1640a-n.

The subject assessment module 1650 can determine the level of effectiveness of each mode or type of stimulation independently, or based on a combination of modes or types of stimulation. A combination of modes of stimulation can refer to transmitting signals from different modes of stimulation at the same or substantially similar time. A combination of modes of stimulation can refer to transmitting signals from different modes of stimulation in an overlapping manner. A combination of modes of stimulation can refer to transmitting signals from different modes of stimulation in a non-overlapping manner, but within a time interval from one another (e.g., transmit a signal pulse train from a second mode of stimulation within 0.5 seconds, 1 second, 1.5 seconds, 2 seconds, 2.5 seconds, 3 seconds, 5 seconds, 7 seconds, 10 seconds, 12 seconds, 15 seconds, 20 seconds, 30 seconds, 45 seconds, 60 seconds, 1 minute, 2 minutes 3 minutes 5 minutes, 10 minutes, or other time interval where the effect on the frequency of neural oscillation by a first mode can overlap with the second mode).

The subject assessment module 1650 can aggregate or compile the information and update the profile data structure 1620 stored in data repository 1615. In some cases, the subject assessment module 1650 can update or generate a policy 1625 based on the received information. The policy 1625 or profile information 1620 can indicate which modes or types of stimulation are more likely to have a desired effect on neural stimulation, while reducing side effects.

The stimuli orchestration component 1610 can instruct or cause multiple signaling components 1630a-n to generate, transmit or otherwise provide different types of stimulation or signals pursuant to the policy 1625, profile information 1620 or feedback information detected by feedback components 1640a-n. The stimuli orchestration component 1610 can cause multiple signaling components 1630a-n to generate, transmit or otherwise provide different types of stimulation or signals simultaneously or at substantially the same time. For example, a first signaling component 1630a can transmit a first type of stimulation at the same time as a second signaling component 1630b transmits a second type of stimulation. The first signaling component 1630a can transmit or provide a first set of signals, pulses or stimulation at the same time the second signaling component 1630b transmits or provides a second set of signals, pulses or stimulation. For example, a first pulse from a first signaling component 1630a can begin at the same time or substantially the same time (e.g., 1%, 2%, 3%, 4%, 5%, 6%, 7%, 10%, 15%, 20%) as a second pulse from a second signaling component 1630b. First and second pulses can end at the same time or substantially same time. In another example, a first pulse train can be transmitted by the first signaling component 1630a at the same or substantially similar time as a second pulse train transmitted by the second signaling component 1630b.

The stimuli orchestration component 1610 can cause multiple signaling components 1630a-n to generate, transmit or otherwise provide different types of stimulation or signals in an overlapping manner. The different pulses or pulse trains may overlap one another but may not necessary being or end at a same time. For example, at least one pulse in the first set of pulses from the first signaling component 1630a can at least partially overlap, in time, with at least one pulse from the second set of pulses from the second signaling component 1630b. For example, the pulses can straddle one another. In some cases, a first pulse train transmitted or provided by the first signaling component 1630a can at least partially overlap with a second pulse train transmitted or provided by the second signaling component 1630b. The first pulse train can straddle the second pulse train.

The stimuli orchestration component 1610 can cause multiple signaling components 1630a-n to generate, transmit or otherwise provide different types of stimulation or signals such that they are received, perceived or otherwise observed by one or more regions or portions of the brain at the same time, simultaneously or at substantially the same time. The brain can receive different modes of stimulation or types of signals at different times. The duration of time between transmission of the signal by a signaling component 1630a-n and reception or perception of the signal by the brain can vary based on the type of signal (e.g., visual, auditory), parameter of the signal (e.g., velocity or speed of the wave, amplitude, frequency, wavelength), or distance between the signaling component 1630a-n and the nerves or cells of the subject configured to receive the signal (e.g., eyes or ears). The stimuli orchestration component 1610 can offset or delay the transmission of signals such that the brain perceives the different signals at the desired time. The stimuli orchestration component 1610 can offset or delay the transmission of a first signal transmitted by a first signaling component 1630a relative to transmission of a second signal transmitted by a second signaling component 1630b. The stimuli orchestration component 1610 can determine an amount of an offset for each type of signal or each signaling component 1630a-n relative to a reference clock or reference signal. The stimuli orchestration component 1610 can be preconfigured or calibrated with an offset for each signaling component 1630a-n.

The stimuli orchestration component 1610 can determine to enable or disable the offset based on the policy 1625. For example, the policy 1625 may indicate to transmit multiple signals at the same time, in which case the stimuli orchestration component 1610 may disable or not use an offset. In another example, the policy 1625 may indicate to transmit multiple signals such that they are perceived by the brain at the same time, in which case the stimuli orchestration component 1610 may enable or use the offset.

In some embodiments, the stimuli orchestration component 1610 can stagger signals transmitted by different signaling components 1630a-n. For example, the stimuli orchestration component 1610 can stagger the signals such that the pulses from different signaling components 1630a-n are non-overlapping. The stimuli orchestration component 1610 can stagger pulse trains from different signaling components 1630a-n such that they are non-overlapping. The stimuli orchestration component 1610 can set parameters for each mode of stimulation or signaling component 1630a-n such that the signals they are non-overlapping.

Thus, the stimuli orchestration component 1610 can set parameters for signals transmitted by one or more signaling components 1630a-n such that the signals are transmitted in a synchronously or asynchronously, or perceived by the brain synchronously or asynchronously. The stimuli orchestration component 1610 can apply the policy 1625 to available signaling components 1630a-n to determine the parameters to set for each signaling component 1630a-n for the synchronous or asynchronous transmission. The stimuli orchestration component 1610 can adjust parameters such as a time delay, phase offset, frequency, pulse rate interval, or amplitude to synchronize the signals.

In some embodiments, the NSOS 1605 can adjust or change the mode of stimulation or a type of signal based on feedback received from a feedback component 1640a-n. The stimuli orchestration component 1610 can adjust the mode of stimulation or type of signal based on feedback on the subject, feedback on the environment, or a combination of feedback on the subject and the environment. Feedback on the subject can include, for example, physiological information, temperature, attention level, level of fatigue, activity (e.g., sifting, laying down, walking, biking, or driving), vision ability, hearing ability, side effects (e.g., pain, migraine, ringing in ear, or blindness), or frequency of neural oscillation at a region or portion of the brain (e.g., EEG probes). Feedback information on the environment can include, for example, ambient temperature, ambient light, ambient sound, battery information, or power source.

The stimuli orchestration component 1610 can determine to maintain or change an aspect of the stimulation treatment based on the feedback. For example, the stimuli orchestration component 1610 can determine that the neurons are not oscillating at the desired frequency in response to the first mode of stimulation. Responsive to determining that the neurons are not oscillating at the desired frequency, the stimuli orchestration component 1610 can disable the first mode of stimulation and enable a second mode of stimulation. The stimuli orchestration component 1610 can again determine (e.g., via feedback component 1640a) that the neurons are not oscillating at the desired frequency in response to the second mode of stimulation. Responsive to determining that the neurons are still not oscillating at the desired frequency, the stimuli orchestration component 1610 can increase an amplitude of the signal corresponding to the second mode of stimulation. The stimuli orchestration component 1610 can determine that the neurons are oscillating at the desired frequency in response to increasing the amplitude of a signal corresponding to the second mode of stimulation.

The stimuli orchestration component 1610 can monitor the frequency of neural oscillations at a region or portion of the brain. The stimuli orchestration component 1610 can determine that neurons in a first region of the brain are oscillating at the desired frequency, whereas neurons in a second region of the brain are not oscillating at the desired frequency. The stimuli orchestration component 1610 can perform a lookup in the profile data structure 1620 to determine a mode of stimulation or type of signal that maps to the second region of the brain. The stimuli orchestration component 1610 can compare the results of the lookup with the currently enabled mode of stimulation to determine that a third mode of stimulation is more likely to cause the neurons in the second region of the brain to oscillate at the desired frequency. Responsive to the determination, the stimuli orchestration component 1610 can identify a signaling component 1630a-n configured to generate and transmit signals corresponding to the selected third mode of stimulation, and instruct or cause the identified signaling component 1630a-n to transmit the signals.

In some embodiments, the stimuli orchestration component 1610 can determine, based on feedback information, that a mode of stimulation is likely to affect the frequency of neural oscillation, or unlikely to affect the frequency of neural oscillation. The stimuli orchestration component 1610 can select a mode of stimulation from a plurality of modes of stimulation that is most likely to affect the frequency of neural stimulation or result in a desired frequency of neural oscillation. If the stimuli orchestration component 1610 determines, based on the feedback information, that a mode of stimulation is unlikely to affect the frequency of neural oscillation, the stimuli orchestration component 1610 can disable the mode of stimulation for a predetermined duration or until the feedback information indicates that the mode of stimulation would be effective.

The stimuli orchestration component 1610 can select one or more modes of stimulation to conserve resources or minimize resource utilization. For example, the stimuli orchestration component 1610 can select one or more modes of stimulation to reduce or minimize power consumption if the power source is a battery or if the battery level is low. In another example, the stimuli orchestration component 1610 can select one or more modes of stimulation to reduce heat generation if the ambient temperature is above a threshold or the temperature of the subject is above a threshold. In another example, the stimuli orchestration component 1610 can select one or more modes of stimulation to increase the level of attention if the stimuli orchestration component 1610 determines that the subject is not focusing on the stimulation (e.g., based on eye tracking or an undesired frequency of neural oscillations).

### Neural Stimulation via Visual Stimulation and Auditory Stimulation

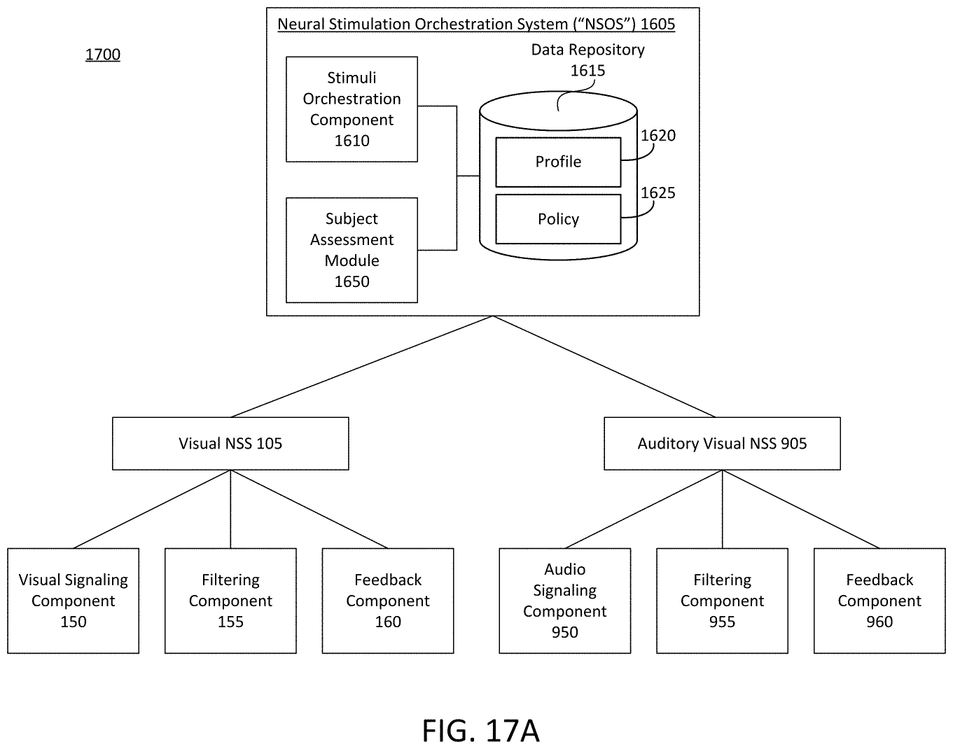

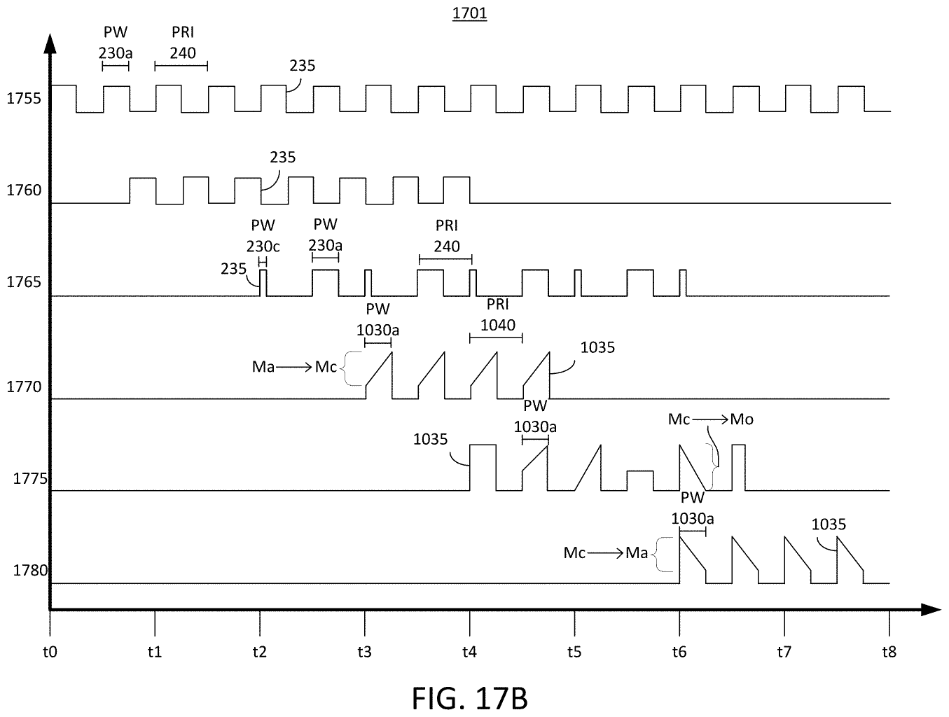

FIG. 17A is a block diagram depicting an embodiment of a system for neural stimulation via visual stimulation and auditory stimulation. The system 1700 can include the NSOS 1605. The NSOS 1605 can interface with the visual NSS 105 and the auditory NSS 905. The visual NSS 105 can interface or communicate with the visual signaling component 150, filtering component 155, and feedback component 160. The auditory NSS 905 can interface or communicate with the audio signaling component 950, filtering component 955, and feedback component 960.

To provide neural stimulation via visual stimulation and auditory stimulation, the NSOS 1605 can identify the types of available components for the neural stimulation session. The NSOS 1605 can identify the types of visual signals the visual signaling component 150 is configured to generate. The NSOS 1605 can also identify the type of audio signals the audio signaling component 950 is configured to generate. The NSOS 1605 can be configured about the types of visual signals and audio signals the components 150 and 950 are configured to generate. The NSOS 1605 can ping the components 150 and 950 for information about the components 150 and 950. The NSOS 1605 can query the components, send an SNMP request, broadcast a query, or otherwise determine information about the available visual signaling component 150 and audio signaling component 950.

For example, the NSOS 1605 can determine that the following components are available for neural stimulation: the visual signaling component 150 includes the virtual reality headset 401 depicted in FIG. 4C; the audio signaling component 950 includes the speaker 1205 depicted in FIG. 12B; the feedback component 160 includes an ambient light sensor 605, an eye tracker 605 and an EEG probe depicted in FIG. 4C; the feedback component 960 includes a microphone 1210 and feedback sensor 1225 depicted in FIG. 12B; and the filtering component 955 includes a noise cancellation component 1215. The NSOS 1605 can further determine an absence of filtering component 155 communicatively coupled to the visual NSS 105. The NSOS 1605 can determine the presence (available or online) or absence (offline) of components via visual NSS 105 or auditory NSS 905. The NSOS 1605 can further obtain identifiers for each of the available or online components.

The NSOS 1605 can perform a lookup in the profile data structure 1620 using an identifier of the subject to identify one or more types of visual signals and audio signals to provide to the subject. The NSOS 1605 can perform a lookup in the profile data structure 1620 using identifiers for the subject and each of the online components to identify one or more types of visual signals and audio signals to provide to the subject. The NSOS 1605 can perform a lookup up in the policy data structure 1625 using an identifier of the subject to obtain a policy for the subject. The NSOS 1605 can perform a lookup in the policy data structure 1625 using identifiers for the subject and each of the online components to identify a policy for the types of visual signals and audio signals to provide to the subject.

FIG. 17B is a diagram depicting waveforms used for neural stimulation via visual stimulation and auditory stimulation in accordance with an embodiment. FIG. 17B illustrates example sequences or a set of sequences 1701 that the stimuli orchestration component 1610 can generate or cause to be generated by one or more visual signaling components 150 or audio signal components 950. The stimuli orchestration component 1610 can retrieve the sequences from a data structure stored in data repository 1615 of NSOS 1605, or a data repository corresponding to NSS 105 or NSS 905. The sequences can be stored in a table format, such as Table 1 below. In some embodiments, the NSOS 1605 can select predetermined sequences to generate a set of sequences for a treatment session or time period, such as the set of sequences in Table 1. In some embodiments, the NSOS 1605 can obtain a predetermined or preconfigured set of sequences. In some embodiments, the NSOS 1605 can construct or generate the set of sequences or each sequence based on information obtained from the subject assessment module 1650. In some embodiments, the NSOS 1605 can remove or delete sequences from the set of sequences based on feedback, such as adverse side effects. The NSOS 1605, via subject assessment module 1650, can include sequences that are more likely to stimulate neurons in a predetermined region of the brain to oscillate at a desired frequency.

The NSOS 1605 can determine, based on the profile information, policy, and available components, to use the following sequences illustrated in example Table 1 provide neural stimulation using both visual signals and auditory signals.

TABLE 1
Audio and Video Stimulation Sequences

| Sequence | | | Signal | Stimulation | | Timing |
|---|---|---|---|---|---|---|
| Identifier | Mode | Signal Type | Parameter | Frequency | | Schedule |
| 1755 | visual | light pulses | Color: red; | 40 | Hz | {t0:t8) |
| | | from a laser | Intensity: low; | | | |
| | | light source | PW: 230a | | | |
| 1760 | visual | checkerboard | color: | 40 | Hz | {t1:t4} |
| | | pattern image | black/white; | | | |
| | | from a tablet | intensity: high; | | | |
| | | display screen | PW: 230a | | | |
| | | light source | | | | |
| 1765 | visual | modulated | PW: | 40 | Hz | {t2:t6} |
| | | ambient light by | 230c/230a; | | | |
| | | a frame with | | | | |
| | | actuated shutters | | | | |
| 1770 | audio | music from | amplitude | 40 | Hz | {t3:t5} |
| | | headphones | variation from | | | |
| | | or speakers | Mₐ to M_{c}; | | | |
| | | connected to an | PW: 1030a | | | |
| | | audio player | | | | |
| 1775 | audio | acoustic or | PW: 1030a; | 39.8 | Hz | {t4:t7} |
| | | audio bursts | frequency | | | |
| | | provided by | variation from | | | |
| | | headphones | M_{c} to Mₒ; | | | |
| | | or speakers | | | | |
| 1780 | audio | air pressure | PW: 1030a; | 40 | Hz | {t6:t8} |

| | |
|---|---|
| generated by a | pressure |
| cochlear air jet | varies from |
| | M_{c} to Mₐ |

As illustrated in Table 1, each waveform sequence can include one or more characteristics, such as a sequence identifier, a mode, a signal type, one or more signal parameters, a modulation or stimulation frequency, and a timing schedule. As illustrated in FIG. 17B and Table 1, the sequence identifiers are 1755, 1760, 1765, 1765, 1770, 1775, and 1760.

The stimuli orchestration component 1610 can receive the characteristics of each sequence. The stimuli orchestration component 1610 can transmit, configure, load, instruct or otherwise provide the sequence characteristics to a signaling component 1630a-n. In some embodiments, the stimuli orchestration component 1610 can provide the sequence characteristics to the visual NSS 105 or the auditory NSS 905, while in some cases the stimuli orchestration component 1610 can directly provide the sequence characteristics to the visual signaling component 150 or audio signaling component 950.

The NSOS 1605 can determine, from the Table 1 data structure, that the mode of stimulation for sequences 1755, 1760 and 1765 is visual by parsing the table and identifying the mode. The NSOS 1605, responsive to determine the mode is visual, can provide the information or characteristics associated with sequences 1755, 1760 and 1765 to the visual NSS 105. The NSS 105 (e.g., via the light generation module 110) can parse the sequence characteristics and then instruct the visual signaling component 150 to generate and transmit the corresponding visual signals. In some embodiments, the NSOS 1605 can directly instruct the visual signaling component 150 to generate and transmit visual signals corresponding to sequences 1755, 1760 and 1765.

The NSOS 1605 can determine, from the Table 1 data structure, that the mode of stimulation for sequences 1770, 1775 and 1780 is audio by parsing the table and identifying the mode. The NSOS 1605, responsive to determine the mode is audio, can provide the information or characteristics associated with sequences 1770, 1775 and 1780 to the auditory NSS 905. The NSS 905 (e.g., via the light generation module 110) can parse the sequence characteristics and then instruct the audio signaling component 950 to generate and transmit the corresponding audio signals. In some embodiments, the NSOS 1605 can directly instruct the visual signaling component 150 to generate and transmit visual signals corresponding to sequences 1770, 1775 and 1780.

For example, the first sequence 1755 can include a visual signal. The signal type can include light pulses 235 generated by a light source 305 that includes a laser. The light pulses can include light waves having a wavelength corresponding to the color red in the visible spectrum. The intensity of the light can be set to low. An intensity level of low can correspond to a low contrast ratio (e.g., relative to the level of ambient light) or a low absolute intensity. The pulse width for the light burst can correspond to pulsewidth 230a depicted in FIG. 2C. The stimulation frequency can be 40 Hz, or correspond to a pulse rate interval (“PRI”) of 0.025 seconds. The first sequence 1655 can run from t₀ to t₈. The first sequence 1655 can run for the duration of the session or treatment. The first sequence 1655 can run while one or more other sequences are other running. The time intervals can refer to absolute times, time periods, number of cycles, or other event. The time interval from t₀ to t₈ can be, for example, 1 minute, 2 minutes, 3 minutes, 4 minutes, 5 minutes, 7 minutes, 10 minutes, 12 minutes, 15 minutes, 20 minutes or more or less. The time interval can be cut short or terminated by the subject or responsive to feedback information. The time intervals can be adjusted based on profile information or by the subject via an input device.

The second sequence 1760 can be another visual signal that begins at t₁ and ends at t₄. The second sequence 1760 can include a signal type of a checkerboard image pattern that is provided by a display screen of a tablet. The signal parameters can include the colors black and white such that the checkerboard alternates black and white squares. The intensity can be high, which can correspond to a high contrast ratio relative to ambient light; or there can be a high contrast between the objects in the checkerboard pattern. The pulse width for the checkerboard pattern can be the same as the pulse width 230a as in sequence 1755. Sequence 1760 can begin and end at a different time than sequence 1755. For example, sequence 1760 can begin at t₁, which can be offset from to by 5 seconds, 10 seconds, 15 seconds, 20 seconds, 20 seconds, 30 seconds, 1 minute, 2 minutes, 3 minutes, or more or less. The visual signaling component 150 can initiate the second sequence 1760 at t₁, and terminate the second sequence at t₄. Thus, the second sequence 1760 can overlap with the first sequence 1755.

While pulse trains or sequences 1755 and 1760 can overlap with one another, the pulses 235 of the second sequence 1760 may not overlap with the pulses 235 of the first sequence 1755. For example, the pulses 235 of the second sequence 1760 can be offset from the pulses 235 of the first sequence 1755 such that they are non-overlapping.

The third sequence 1765 can include a visual signal. The signal type can include ambient light that is modulated by actuated shutters configured on frames (e.g., frames 400 depicted in FIG. 4B). The pulse width can vary from 230c to 230a in the third sequence 1765. The stimulation frequency can still be 40 Hz, such that the PRI is the same as the PRI in sequence 1760 and 1755. The pulses 235 of the third sequence 1765 can at least partially overlap with the pulses 235 of sequence 1755, but may not overlap with the pulses 235 of the sequence 1760. Further, the pulse 235 can refer to block ambient light or allowing ambient light to be perceived by the eyes. In some embodiments, pulse 235 can correspond to blocking ambient light, in which case the laser light pulses 1755 may appear to have a higher contrast ratio. In some cases, the pulses 235 of sequence 1765 can correspond to allowing ambient light to enter the eyes, in which case the contrast ratio for pulses 235 of sequence 1755 may be lower, which may mitigate adverse side effects.

The fourth sequence 1770 can include an auditory stimulation mode. The fourth sequence 1770 can include upchirp pulses 1035. The audio pulses can be provided via headphones or speakers 1205 of FIG. 12B. For example, the pulses 1035 can correspond to modulating music played by an audio player 1220 as depicted in FIG. 12B. The modulation can range from Mₐ to M_{c}. The modulation can refer to modulating the amplitude of the music. The amplitude can refer to the volume. Thus, the NSOS 1605 can instruct the audio signaling component 950 to increase the volume from a volume level Mₐ to a volume level M_{c} during a duration PW 1030a, and then return the volume to a baseline level or muted level in between pulses 1035. The PRI 240 can be 0.025, or correspond to a 40 Hz stimulation frequency. The NSOS 1605 can instruct the fourth sequence 1770 to begin at t₃, which overlaps with visual stimulation sequences 1755, 1760 and 1765.

The fifth sequence 1775 can include another audio stimulation mode. The fifth sequence 1775 can include acoustic bursts. The acoustic bursts can be provided by the headphones or speakers 1205 of FIG. 12B. The sequence 1775 can include pulses 1035. The pulses 1035 can vary from one pulse to another pulse in the sequence. The fifth waveform 1775 can be configured to re-focus the subject to increase the subject's attention level to the neural stimulation. The fifth sequence 1775 can increase the subject's attention level by varying parameters of the signal from one pulse to the other pulse. The fifth sequence 1775 can vary the frequency from one pulse to the other pulse. For example, the first pulse 1035 in sequence 1775 can have a higher frequency than the previous sequences. The second pulse can be an upchirp pulse having a frequency that increases from a low frequency to a high frequency. The third pulse can be a sharper upchirp pulse that has frequency that increases from an even lower frequency to the same high frequency. The fifth pulse can have a low stable frequency. The sixth pulse can be a downchirp pulse going from a high frequency to the lowest frequency. The seventh pulse can be a high frequency pulse with a small pulsewidth. The fifth sequence 1775 can being at t₄ and end at t₇. The fifth sequence can overlap with sequence 1755; and partially overlap with sequence 1765 and 1770. The fifth sequence may not overlap with sequence 1760. The stimulation frequency can be 39.8 Hz.

The sixth sequence 1780 can include an audio stimulation mode. The signal type can include pressure or air provided by an air jet. The sixth sequence can begin at t₆ and end at t₈. The sixth sequence 1780 can overlap with sequence 1755, and partially overlap with sequences 1765 and 1775. The sixth sequence 1780 can end the neural stimulation session along with the first sequence 1755. The air jet can provide pulses 1035 with pressure ranging from a high pressure M_{c} to a low pressure Mₐ. The pulse width can be 1030a, and the stimulation frequency can be 40 Hz.

The NSOS 1605 can adjust, change, or otherwise modify sequences or pulses based on feedback. In some embodiments, the NSOS 1605 can determine, based on the profile information, policy, and available components, to provide neural stimulation using both visual signals and auditory signals. The NSOS 1605 can determine to synchronize the transmit time of the first visual pulse train and the first audio pulse train. The NSOS 1605 can transmit the first visual pulse train and the first audio pulse train for a first duration (e.g., 1 minute, 2 minutes, or 3 minutes). At the end of the first duration, the NSOS 1605 can ping an EEG probe to determine a frequency of neural oscillation in a region of the brain. If the frequency of oscillation is not at the desired frequency of oscillation, the NSOS 1605 can select a sequence out of order or change the timing schedule of a sequence.

For example, the NSOS 1605 can ping a feedback sensor at ti. The NSOS 1605 can determine, at ti, that neurons of the primary visual cortex are oscillating at the desired frequency. Thus, the NSOS 1605 can determine to forego transmitting sequences 1760 and 1765 because neurons of the primary visual cortex are already oscillating at the desired frequency. The NSOS 1605 can determine to disable sequences 1760 and 1765. The NSOS 1605, responsive to the feedback information, can disable the sequences 1760 and 1765. The NSOS 1605, responsive to the feedback information, can modify a flag in the data structure corresponding to Table 1 to indicate that the sequences 1760 and 1765 are disabled.

The NSOS 1605 can receive feedback information at t₂. At t₂, the NSOS 1605 can determine that the frequency of neural oscillation in the primary visual cortex is different from the desired frequency. Responsive to determining the difference, the NSOS 1605 can enable or re-enable sequence 1765 in order to stimulate the neurons in the primary visual cortex such that the neurons may oscillate at the desired frequency.

Similarly, the NSOS 1605 can enable or disable audio stimulation sequences 1770, 1775 and 1780 based on feedback related to the auditory cortex. In some cases, the NSOS 1605 can determine to disable all audio stimulation sequences if the visual sequence 1755 is successfully affecting the frequency of neural oscillations in the brain at each time period t₁, t₂, t₃, t₄, t₅, t₆, t₇, and t₈. In some cases, the NSOS 1605 can determine that the subject is not paying attention at t4, and go from only enabling visual sequence 1755 directly to enabling audio sequence 1755 to re-focus the user using a different stimulation mode.

### Method for Neural Stimulation via Visual Stimulation and Auditory Stimulation

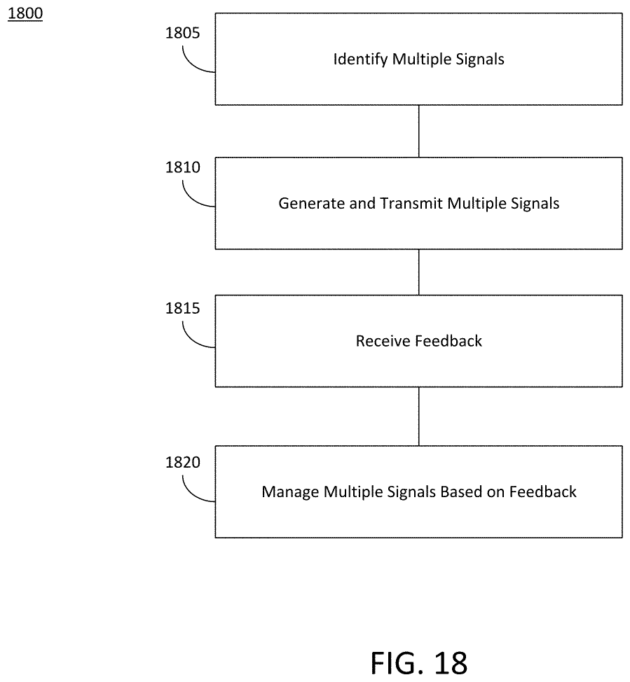

FIG. 18 is a flow diagram of a method for neural stimulation via visual stimulation and auditory stimulation in accordance with an embodiment. The method 180 can be performed by one or more system, component, module or element depicted in FIGS. 1-17B, including, for example, a neural stimulation orchestration component or neural stimulations system. In brief overview, the NSOS can identify multiple modes of signals to provide at block 1805. At block 1810, the NSOS can generate and transmit the identified signals corresponding to the multiple modes. At 1815 the NSOS can receive or determine feedback associated with neural activity, physiological activity, environmental parameters, or device parameters. At 1820 the NSOS can manage, control, or adjust the one or more signals based on the feedback.

While this specification contains many specific implementation details, these should not be construed as limitations on the scope of any disclosure or of what can be claimed, but rather as descriptions of features specific to particular embodiments of particular aspects. Certain features described in this specification in the context of separate embodiments can also be implemented in combination in a single embodiment. Conversely, various features described in the context of a single embodiment can also be implemented in multiple embodiments separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’.

Thus, particular embodiments of the subject matter have been described. In some cases, the actions recited in the claims can be performed in a different order and still achieve desirable results. In addition, the processes depicted in the accompanying figures do not necessarily require the particular order shown, or sequential order, to achieve desirable results.

The present technology, including the systems, methods, devices, components, modules, elements or functionality described or illustrated in, or in association with, the figures can treat, prevent, protect against or otherwise affect brain atrophy and disorders, conditions and diseases associated with brain atrophy.

### Combination Therapies

In one aspect, the present disclosure provides combination therapies comprising the administration of one or more additional therapeutic regimens in conjunction with methods described herein. In some embodiments, the additional therapeutic regimens are directed to the treatment or prevention of the disease or disorder targeted by methods of the present technology.

In some embodiments, the additional therapeutic regimens comprise administration of one or more pharmacological agents that are used to treat or prevent disorders targeted by methods of the present technology. In some embodiments, methods of the present technology facilitate the use of lower doses of pharmacological agents to treat or prevent targeted disorders. In some embodiments, the additional therapeutic regimens comprise non-pharmacological therapies that are used to treat or prevent disorders targeted by methods of the present technology such as, but not limited to, cognitive or physical therapeutic regimens.

In some embodiments, a pharmacological agent is administered in conjunction with therapeutic methods described herein. In some embodiments, the pharmacological agent is directed to inducing a relaxed state in a subject administered methods of the present technology. In some embodiments, the pharmacological agent is directed to inducing a heightened state of awareness in a subject administered methods of the present technology. In some embodiments, the pharmacological agent is directed to modulating neuronal and/or synaptic activity. In some embodiments, the agent promotes neuronal and/or synaptic activity. In some embodiments, the agent targets a cholinergic receptor. In some embodiments, the agent is a cholinergic receptor agonist. In some embodiments, the agent is acetylcholine or an acetylcholine derivative. In some embodiments, the agent is an acetylcholinesterase inhibitor.

In some embodiments, the agent inhibits neuronal and/or synaptic activity. In some embodiments, the agent is a cholinergic receptor antagonist. In some embodiments, the agent is an acetylcholine inhibitor or an acetylcholine derivative inhibitor. In some embodiments, the agent is acetylcholinesterase or an acetylcholinesterase derivative.

### EXAMPLES

### Example 1

### Human Clinical Study of Safety, Efficacy, and Results of Treatment

### Introduction

Gamma sensory stimulation is a novel therapeutic intervention for AD that utilizes auditory and visual stimuli to evoke specific neural activity. Based on preclinical studies, repeated daily exposure to sensory-evoked gamma oscillations results in multiple beneficial effects on Alzheimer's pathophysiology in transgenic mouse models. These effects include reduced production of soluble and insoluble A-beta, microglia-mediated phagocytosis of amyloid plaques, reduced tau-hyperphosphorylation, and improved cognitive function.

### Objectives

A sensory stimulation system was used in this study. The sensory stimulation system comprised a handheld controller, an eyeset for visual stimulation, and headphones for auditory stimulation that work together to deliver precisely timed, non-invasive stimulation to induce steady-state gamma brainwave activity. The stimuli occur at a pulse repetition frequency of 40 Hz to induce synchronized gamma oscillations in at least one region of the brain. The objective of this study was to assess the safety, tolerability, and efficacy of long-term, daily use of gamma sensory stimulation therapy on cognition, functional ability, and biomarkers in a mild-to-moderate AD population via a prospective clinical study.

### Methods and Study Design

Methods and Study Design: A clinical study was done. The clinical study was a multi-center, randomized controlled trial evaluating daily gamma sensory stimulation received at home for a 6-month treatment period. Subjects included in the study were adults 50 years and older with a clinical diagnosis of mild to moderate AD (MMSE: 14-26, inclusive), a reliable care partner, and successful tolerance and entrainment screening via EEG. Key exclusion criteria included profound hearing or visual impairment, use of memantine, major psychiatric illness, clinically relevant history of seizure, or contraindication to imaging studies.

A total of 135 patients were assessed for eligibility to participate in the study. Patients were first given a screening EEG, and then split into groups. One group was a sham control group that was not given treatment; the other was a group that was subjected to 1 hour of therapy, which involved subjecting the subject to audio and visual stimulation at a frequency of 40 Hz per day. Of those assessed for eligibility, 76 were randomized between the active treatment and sham control. Forty-seven of the randomized patients were allocated to the active group and 29 were allocated to the sham group. Of the active group, two patients withdrew prior to therapy and three had no post-baseline efficacy and were not included in the modified intent to treat (mITT) population. In sham group, one patient received active treatment and was not in the sham population. Completers included 33 patients in the active group and 28 in the sham group, with 10 early discontinuations in the active group. Seven of those discontinuations were due to consent withdraw and 23 were attributed to adverse events, whereas in the sham group, only six withdrew consent and one discontinued as a result of adverse events.

The study employed various clinical outcome assessment scales to assess cognitive decline or dysfunction. These included the Neuropsychiatric Inventory (NPI), Clinical Dementia Rating-Sum of Boxes (CDR-sb), the Clinical Dementia Rating-Global Score (CDR global), the Mini-Mental State Exam (MMSE), the Alzheimer's Disease Assessment Scale-Cognitive Subscale-14 (ADAS-Cog14), and a variation of the Alzheimer's Disease Composite Score (ADCOMS) as optimized for patients with mild or moderate Alzheimer's Disease. NPI examines 12 sub-domains of behavioral functioning: delusions, hallucinations, agitation/aggression, dysphoria, anxiety, euphoria, apathy, disinhibition, irritability/lability, and aberrant motor activity, night-time behavioral disturbances, and appetite and eating abnormalities. The NPI can be used to screen for multiple types of dementia, and it involves giving the caregiver of a subject the questions and then, based on the answers, rating the frequency of the symptoms, their severity, and the distress the symptoms cause on a three, four, and five-point scale, respectively.

CDR global is calculated based on testing performed for six different cognitive and behavioral domains: memory, orientation, judgment and problem solving, community affairs, home and hobbies performance, and personal care. To test these areas, an informant is given a set of questions about a subject's memory problem, judgment and problem-solving ability of the subject, community affairs of the subject, home life and hobbies of the subject, and personal questions related to the subject. The subject is given another set of questions that includes memory-related questions, orientation-related questions, and questions about judgment and problem-solving ability. The CDR global score is calculated based on the results of those questions, and it is measured using a scale of 0 to 3, with 0 representing no dementia, 0.5 indicating very mild dementia, 1 indicating mild dementia, 2 indicating moderate dementia/cognitive impairment, and 3 indicating severe dementia/cognitive impairment. CDR-sb is a clinical outcome assessment that looks at functional impact of cognitive impairment: memory, executive function, instrumental and basic activities of daily living and assesses them based on interviews with an informant and the patient. The CDR-sb score is based on assessment of items including memory, orientation, judgment and problem solving, community affairs, home and hobbies, and personal care. The CDR-sb is scored from 0 to 18, with higher scores representing greater severity of cognitive and functional impairment.

The MMSE looks at 11 items to assess memory, language, praxis and executive function based on a cognitive assessment of the patient. Items assessed include registration, recall, constructional praxis, attention and concentration, language, orientation time, and orientation place. The MMSE is scaled from 0 to 30, with higher scores representing lower severity of cognitive dysfunction. The ADAS-Cog14 assesses memory, language, praxis and executive function. The score is based on a cognitive assessment of the patient and assesses fourteen items: spoken language, maze, comprehension spoken language, remembering word recognition test instructions, ideational praxis, commands, naming, word finding difficulty, constructional praxis, orientation, digit cancellation, word recognition, word recall, and delayed recall. A score is based on points allocated to each item, and the maximum total score is 90, with higher numbers indicating greater severity of cognitive dysfunction. The Alzheimer's Disease Composite Score (ADCOMS) considers items from all of the above-discussed scores: items from Alzheimer's Disease Assessment Scale-cognitive subscale items, MMSE items, and all of the CDR-sb items. ADCOMS combines portions of the ADAS-cog, Clinical Dementia Rating (CDR) scale, and MMSE that have been shown to change the most over time in people who do not have functional impairment yet. MADCOMS, which was used in the present example, optimizes the scale instead by combining items more significant for mild and moderate dementia.

The study design involved primary efficacy endpoints of MADCOMS, ADAS-cog14, and CDR-sb. Unlike ADCOMS, MADCOMS is optimized for patients with moderate or mild Alzheimer's Disease. These were optimized for AD-specific decline. A separate optimization was done for moderate and mild AD. Secondary efficacy endpoints consisted of ADCS-ADL, ADCOMs (adjusted), MMSE, CDR-global score and the Neuropsychiatric Inventory (NPI). Of the secondary endpoints, ADCS-ADL was measured monthly and MMSE was measured at the last time point.

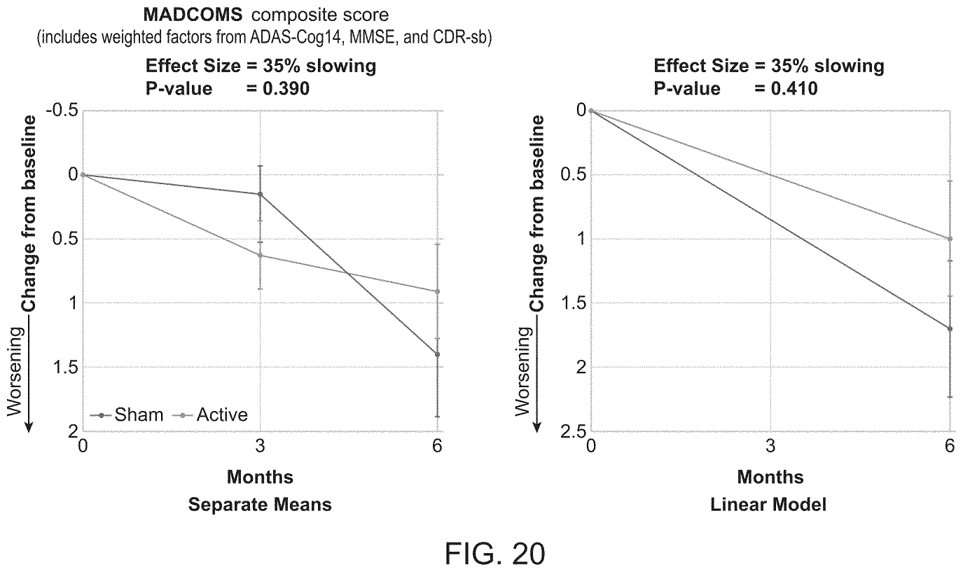

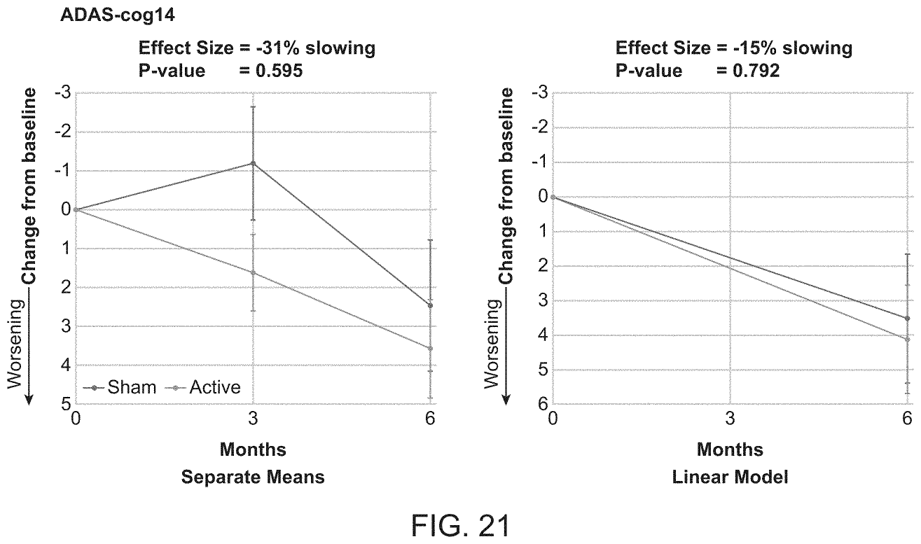

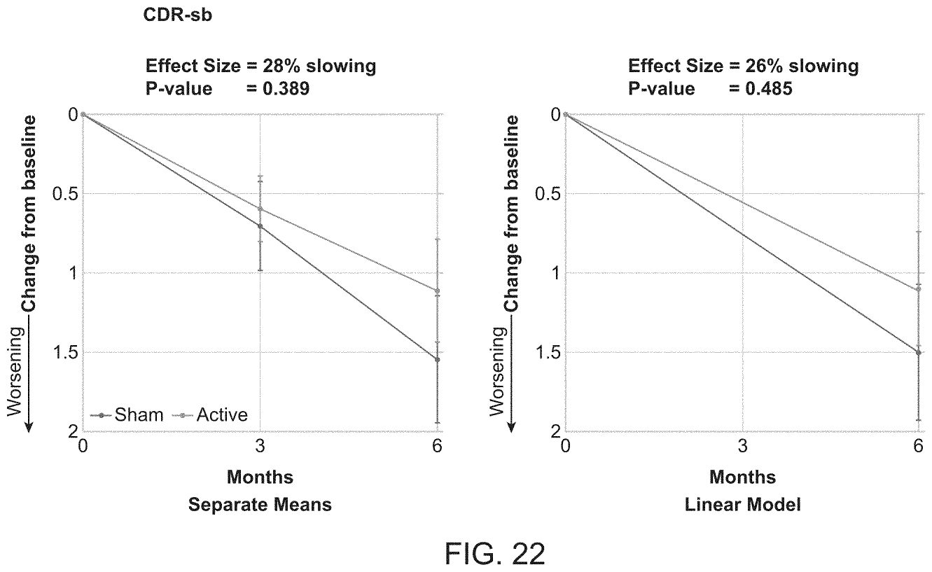

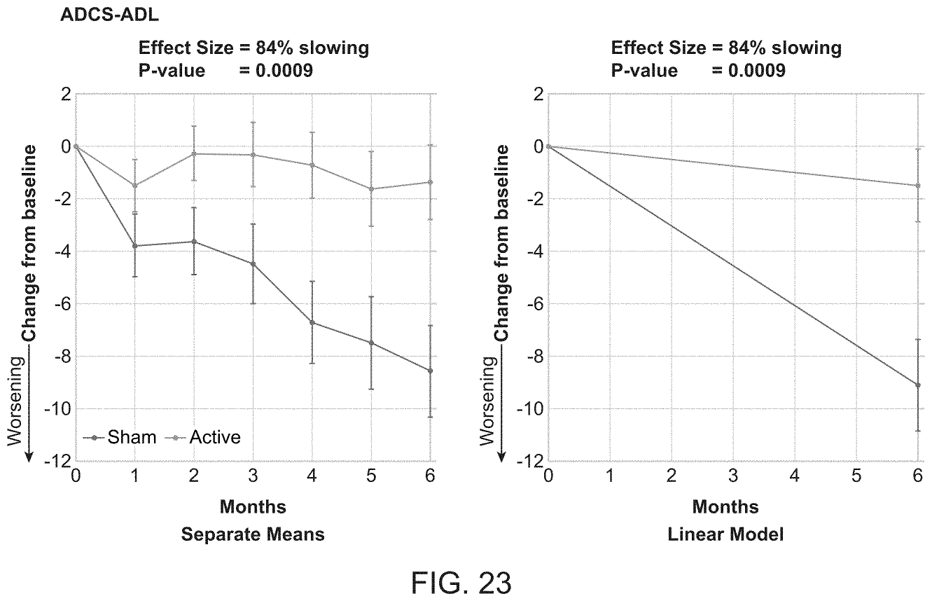

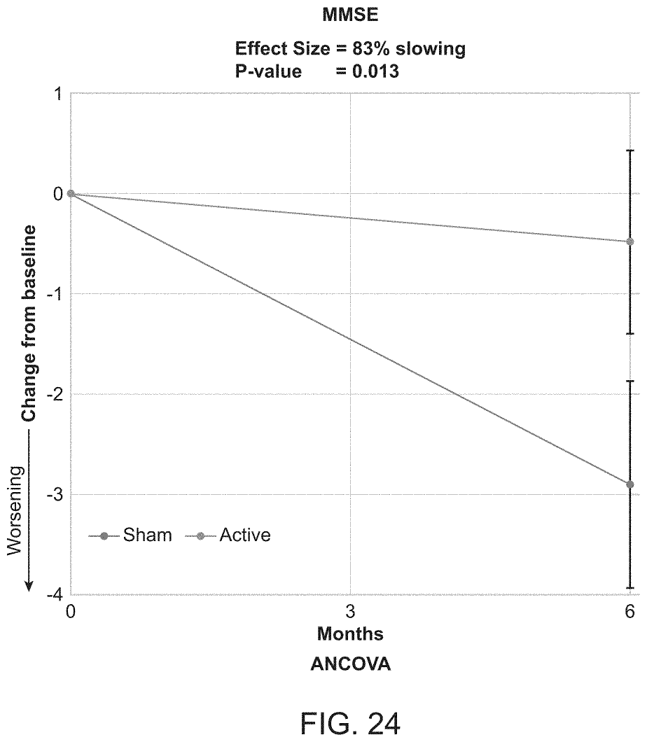

The efficacy endpoints were analyzed by applying a linear model of analysis and/or a separate means model of analysis. The linear model of analysis involved employing a linear fit model to determine a value at T0 based on the difference from baseline in conditions at the end of the study. The seprate means analysis employed estimates of mean values at each assess timepoint, which was either a monthly timepoint or at three and six months after treatment began, depending on the score that was being analyzed. In evaluating MADCOMS composite score, for example, the separate means analysis was applied using mean values that were estimated at three and six months. The linear model was applied by using the estimates of treatment difference at the end of the study and connecting a straight line to 0. Similar models were used for the other efficacy endpoints. FIGS. 20, 21, 22, 23, and 24 show the various linear and separate means models generated for these endpoints.

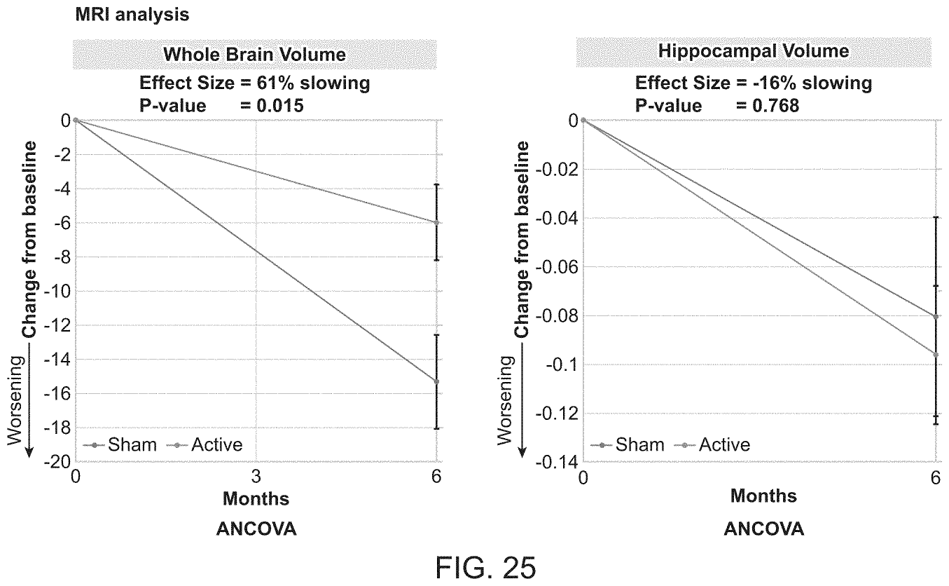

To assess biomarkers, researches used MRI, volumetric analyses, EEG, Amyloid positron emission tomography (PET), actigraphy, and plasma biomarkers. The study employed structural MRIs, taken before any treatment began and at the end of the sixth months and assessed these for volume-base morphology. Volumetric changes for the hippocampus, lateral ventricles, whole cortex (cerebral cortical gray matter) and whole brain (cerebrum and cerebellum) were determined and the rate of atrophy was compared for active and sham groups using a linear model, as demonstrated in FIG. 25.

To analyze for safety and tolerability, researchers looked for adverse events and presence of amyloid related imaging abnormalities (ARIA) on MRI. Safety was evaluated by MRI, a suicidality scale, and physical and neurological exams at baseline, 12-, and 24-weeks, and by monthly assessments of adverse events (AEs) during the trial. Tolerability was measured by device use data, a daily diary, and user experience interviews. Symptomatic changes were assessed daily via a diary, monthly via Alzheimer's Disease Cooperative Study-Activities of Daily Living (ADCS-ADL), Quality of Life, and care partner burden scales, quarterly via the Alzheimer's Disease Assessment Scale cognitive subscale (ADAS-Cog14), Neuropsychiatric Inventory, Clinical Dementia Rating sum of boxes (CDR-sb), and bi-annually via Clock Drawing Test, and the Mini-Mental State Examination (MMSE). Plasma biomarkers, EEG recordings and brain volumetric changes (assessed by MRI), including whole brain, lateral ventricle, occipital and temporal lobe volumes and temporal lobe composite cortical thickness were assessed at baseline and after 3 and 6-months of treatment. APOE status was characterized at baseline. Actigraphy devices were worn continuously throughout the trial to assess daytime and nighttime activity. Therapy adherence was also analyzed. Blinding effectiveness for subjects, care partners, and assessors were prospectively analyzed by assessing baseline and follow up ascertainment of whether the care partner, assessor, or patient thought the patient was on active or sham treatment.

In addition, the study evaluated the effect of stimuli on volume loss in occipital cortical lobe and loss in occipital cortical thickness in a smaller subset of patients over a 6-month period. The completers included 24 patients in the active group and 17 in the sham group. There were 3 and 5 early discontinuations in the active group before 3-month and 6-month time point, respectively, and 4 early discontinuations in the sham group before 3-month. The volume loss and thickness loss were measured by structural MR imaging and illustrated by least-squares mean change at 3-month and 6-month time point from baseline (Day 0) in both sham and treatment groups. The Delta (±SE) at 3-month and 6-month as well as p-values were provided.

### Analysis

For the MADCOMS composite scores, both means of analysis demonstrated 35% slowing in decline rate, indicating that the active group progressed less than the placebo arm over the six-month study. When a linear and means analysis were both employed, the sham group was slightly favored, but non-significantly. When these two separate means analyses were applied to the ADAS-cog14 data, both slightly favored the sham group, although not in a statistically significant manner. When CDR-sb results were analyzed, the mean-estimate model found a 28% slowing rate, whereas the linear extraction showed a 26% slowing rate, but the comparisons were not statistically significant.

Of the secondary endpoints, ADCS-ADL was measured monthly and MMSE was measured at the last time point. When analyzing ADCS-ADL values, the first analysis model employed used estimates for each month and showed 84% slowing over the 6-month time period. The linear fit model was again employed, and the same 84% slowing was found. When analyzing MMSE values, an 83% slowing was identified.

### Results

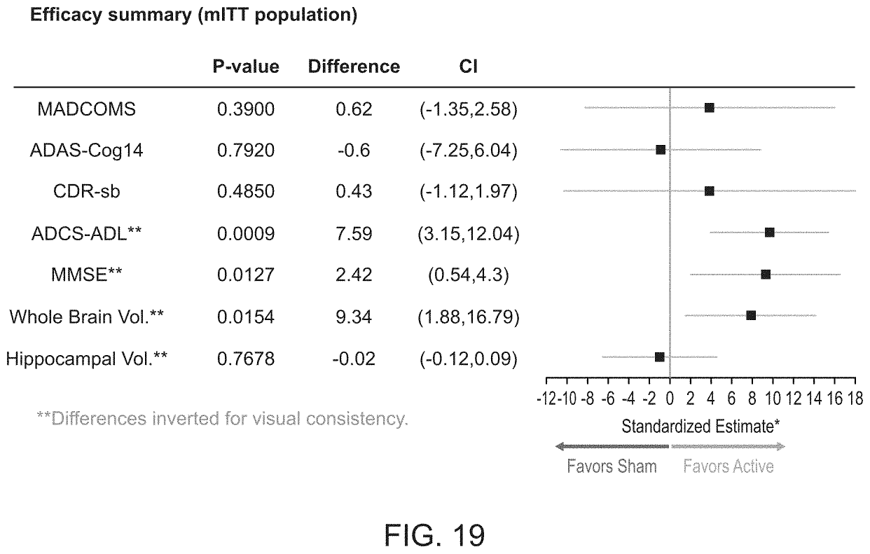

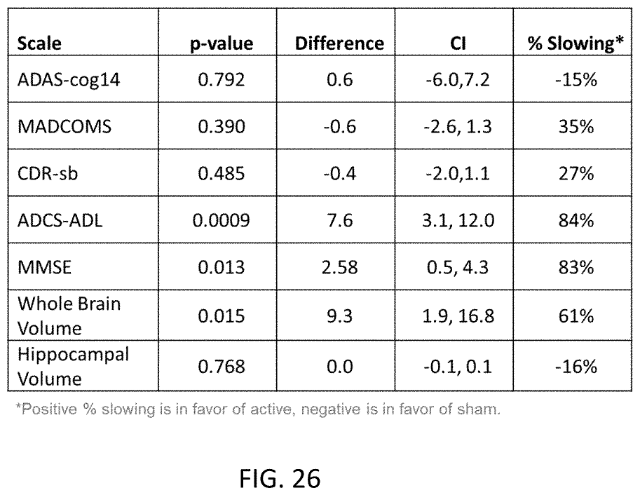

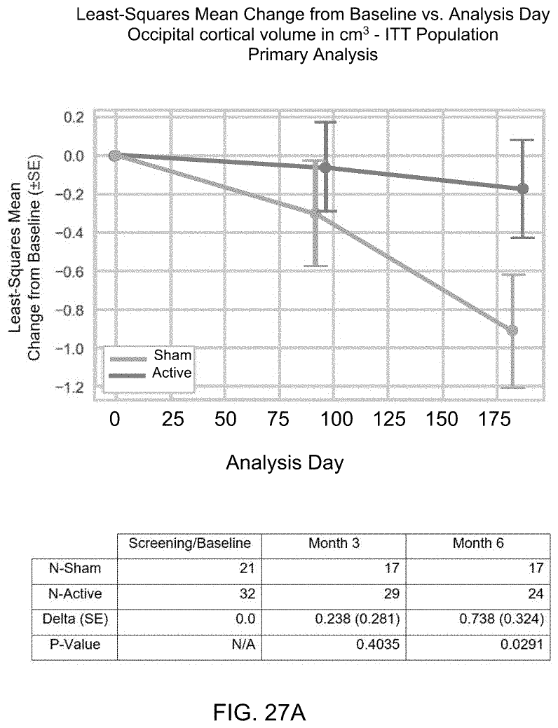

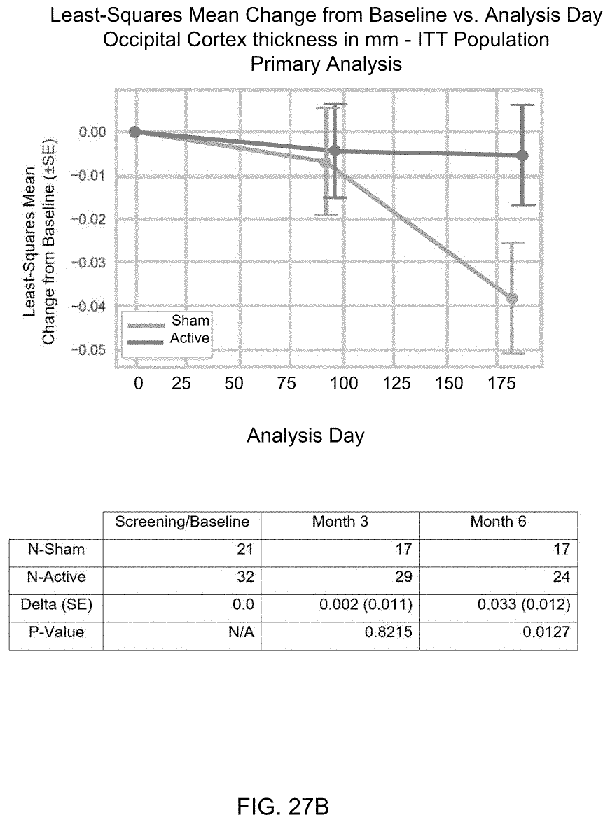

FIGS. 19 and 26 summarize the efficacy findings of the study. Following informed consent and screening, a total of 76 subjects were randomized between the active treatment and sham control. The safety population for the study included 74 subjects who received at least one treatment, and the modified intent to treat (mITT) population included a total of 70 subjects, 53 of whom completed the 6-month study, which form the basis for analysis of outcome measures. FIG. 27A and FIG. 27B show result for occipital cortical volume change and occipital cortex thickness change in both sham and active groups.

### Demographic and Baseline Characteristics

In terms of demographic and baseline characteristics of the mITT population, following randomization, the populations were balanced across gender, baseline MMSE, ApoE4 status, activities of daily living (ADL), and PET amyloid standardized uptake value ratio (SUVR) status; imbalances between the two groups were observed in age, ADAS-Cog11, and CDR-sb scores at baseline. Statistical models included covariates for age and MMSE at baseline.

### Safety and Tolerability

Non-invasive gamma sensory stimulation was safe and well-tolerated in the mild and moderate AD subjects. The active group had a lower rate of treatment emergent adverse events (TEAE) than the sham group (67% vs 79%).

Treatment related AEs (TRAEs) deemed definitely, probably, and possibly related to the therapy were elevated in the active group versus the sham group (41% vs 32%). One treatment related SAE was noted in the active group for a patient hospitalized for wandering while their care partner was located; this subject discontinued the study subsequently. Of the randomized subjects, withdraw rates were similar between both groups (active 28%, sham 29%) including withdraw rates due to an adverse event (active 7%, sham 7%). TEAEs that occurred more often in the active group are tinnitus, delusions, broken bone. TEAEs that occurred more often in the sham group are upper respiratory infection, confusion, anxiety and dizziness.

### Clinical Assessments

Over the treatment period of 6-months, subjects were evaluated in-clinic and via phone visits for cognitive, functional, and biomarker changes on multiple measures.

The primary efficacy endpoints demonstrated effects favoring the active group on the MADCOMS (35% slowing; n.s.) and CDR-sb (27%; n.s.) and favoring the sham group on the ADAS-cog14 (-15% slowing; n.s.). MADCOMS initially leaned in favor of active group, but the results were not statistically different. ADAS-cog14 was slightly in favor of the sham group but not statistically different. CDR-sb was also in favor of the active group, but the difference was not significant, as shown by the p-values that ranged between 0.39 and 0.7920.

Selected secondary endpoints demonstrated significant effects favoring the treatment (active) group. The active group had significant benefit on functional ability as measured by the ADCS-ADL (p=0.0009), which represented an 84% slowing of decline and a treatment difference of 7.59 points over the six-month duration of the trial (FIG. 2). Further analysis of data resulted in more precise p-values. Further analysis showed that, over the 6-month treatment period, changes in ADCS-ADL scores were statistically significant between the sham and treatment groups, indicating a 78% slowing in functional decline by treatment (P<0.0003). The active group demonstrated significant benefit on the MMSE (ANCOVA p=0.013), which represented an 83% slowing in the rate of decline versus the sham group and a treatment difference of 2.42 points.

Among clinical instruments assessing cognitive and functional abilities, ADCS-ADL and MMSE scores demonstrated the most effective outcomes of the therapy. Other independent cognitive tests demonstrated a diminished cognitive decline in the treatment group compared to the sham group, although differences were not statistically significant. The outcomes of MADCOMs, ADAS-cog 14 and CDR-sb were not statistically different between groups. Nighttime active durations in the treatment group were significantly (p<0.03) reduced in the second 3 months compared to the first 3-months and the opposite change was observed in the sham group.

### Biomarker Changes—MRI

Structural MR imaging was analyzed for volume-base morphometry using an automated image processing pipeline (Biospective, Montreal, Canada). Volumetric changes of the hippocampus, lateral ventricles, whole cortex (cerebral cortical gray matter) and whole brain (cerebrum and cerebellum, no CSF) for each subject were determined; no manual corrections were performed. No significant benefit on hippocampal volume was determined. Statistically significant benefit favoring the active group (p=0.0154) on whole brain volume (WBV) was established, representing a 61% slowing compared to the sham group progression. The treatment value for the active group was 9.34 cm³. Quantitative MRI analysis revealed that whole brain volume loss in the treatment group was 0.6%, whereas in the sham group this value was 1.5%, (comparable to the historic value of 1.12%), demonstrating a significant, 63% reduction in brain atrophy (p<0.01) by gamma sensory stimulation over a 6-month period in this patient population.

Additionally, the changes in occipital cortical volume and occipital cortex thickness were determined. The Delta (SE) for occipital cortical volume change (in cm³) at 3-month and 6-month is 0.238(0.281) and 0.738(0.324). The Delta (SE) for occipital cortex thickness change (in mm) at 3-month and 6-month is 0.238(0.281) and 0.738(0.324). The results at 6-month suggest a significant reduction in loss of occipital lobe volume (p=0.0291) and occipital cortical thickness (p=0.0217) in treatment group compared to the sham group (FIGS. 27A-B).

Significant correlations in brain volumetric changes have been observed, including positive correlation between changes in occipital cortex and occipital lobe volume, and similarly, between changes in temporal cortex thickness and temporal lobe in both active treatment and sham groups. As it can be expected, decrease in whole brain volume was significantly correlated with increase in lateral ventricle volume in both active treatment and sham groups. Furthermore, only in the active treatment group, there were significant correlations between changes in temporal and occipital lobe volumes and between temporal and occipital cortical thickness. Changes in whole brain cortical thickness correlated with temporal and occipital cortexes thickness and temporal lobe volume. The correlations between temporal and occipital lobe volume/cortical thickness only in active treatment group indicate a direct effect of sensory-evoked gamma oscillation on these structures.

### Conclusions

Gamma sensory stimulation was safe and well tolerated. Two of three primary efficacy outcomes (MADCOMS, CDR-sb) favored the active group but did not reach significance. Selected secondary endpoints demonstrated that active treatment with gamma sensory stimulation therapy led to significant benefits in the ability to perform activities of daily living via the ADCS-ADL and cognition via the MMSE, representing important treatment and management objectives for AD patients. Quantitative MR analysis demonstrated slowing of brain atrophy as measured by whole brain volume in the active group. The combined clinical and biomarker findings suggest beneficial effects of gamma sensory stimulation for AD subjects may be facilitated via differentiated pathways. These surprising results indicates that the gamma sensory stimulation may be used to treat a range of diseases and disorders that cause or are caused by brain atrophy.

### Example 2

### Methods and Devices of the Present Technology for the Prevention or Treatment of Huntington's Disease

This example demonstrates the use of methods and devices of the present technology in the prevention or treatment of Huntington's Disease (HD) utilizing a BACHD transgenic rodent model and human subjects.

Animal Models

Studies are conducted using transgenic rats carrying a bacterial artificial chromosome (BAC) which contains the full-length HTT genomic sequence with 97 CAG/CAA repeats and all regulatory elements (BACHD) in rat.

Alternatively, studies are conducted using the FVB/N-Tg(HTT*97Q)IX /J BACHD murine model, commercially available (The Jackson Laboratory, Bar Harbor, Me., USA).

BACHD transgenic rodents display a robust, early onset and progressive HD-like phenotype including motor deficits and anxiety-related symptoms. Accordingly, the BACHD transgenic rodent model is useful to evaluate efficacy of HD therapies.

To demonstrate methods of prevention in BACHD transgenic rodent model, subjects are administered methods of the present technology before the development of symptoms or pathologies of HD. To demonstrate methods of treatment in BACHD transgenic rodent model, subjects are administered methods of the present technology subsequent to the development of symptoms or pathologies of HD. Symptoms and/or pathologies of HD may be assessed at predetermined time points; the assessment(s) can include, but are not limited to, a behavioral assessment, the rotarod test, the footprint test, elevated plus maze, locomotor activity and food intake, light electron and immunohistochemistry analysis and quantitative assessment of morphological changes in the striosome compartment.

Behavioral assessment: Subjects are group-housed with mixed genotypes in a constant temperature-humidity room (22±1° C., 55±10% relative humidity) with a 12 h light/dark cycle (lights on/off at 2:00 A.M./P.M.). Food and water are provided ad libitum. All behavioral tests are performed with male rodents during the dark (active) phase. Control animals are an equal mix of wild-type (WT) littermates from both lines.

Rotarod Test: Rotarod experiments (Accelerated rotarod for rodents 7750, Ugo Basile) are used to measure forelimb and hindlimb motor coordination. BACHD transgenic rodents and WT littermates are trained on 3 consecutive days with four trials per day. Directly after training they are tested on 2 consecutive days with two trials per day and an interval of 1 h between individual trials. During the training period, the rodents are placed on the rotating rod at a constant speed of 12 rpm for 2 min. Rodents are returned to the rod after falling during the training period up to 10 falls per trial. Individual tests are assessed for a maximum of 5 min with accelerodenting speed from 4 to 40 rpm over a period of 4 min, and the latencies to fall are recorded. Rodents from the same cohort (n=12) are tested every month from 1 to 15 months of age.

Footprint Test: A footprint test is used to analyze gait abnormalities in BACHD rodents. BACHD transgenics and WT littermates are evaluated at 14 months of age (n=11). On the day before testing, the rodents are trained in three sessions. The best performance out of three tests with each rodent is selected for data analysis. The stride width of the hindpaw, the step length (left paw to left paw), and the overlap (distance between front and hindpaw) are measured in three consecutive steps, and the average is taken for further analysis.

Elevated Plus Maze: An elevated plus maze is used to assess the anxiety of BACHD rodents. To eliminate the possibility of habituation effects, different cohorts of rodents are tested at 1 (n=13 per genotype), 4 (n=13 per genotype), and 12 (TG5:WT=8:11) months of age as described previously. Rodents are placed at the center of an elevated plus maze (with two open and two closed arms) facing an open arm and are monitored for 5 min. The time spent in the open arms is recorded as a percentage of the total time for analysis.

Locomotor Activity and Food Intake: Rodents are monitored using the PhenoMaster system (TSE Systems), which represents a modular setup that screens rodents in a home cage-like environment for their ambulatory activity and rearing, as well as feeding and drinking behavior. Activity detection is achieved using infrared sensor pairs arranged in horizontal (x,y level for ambulatory activity) and vertical (z level for rearing) strips. Food and water consumption are recorded by two weighting sensors. The same cohort of animals (TG5:TG9:WT=16:19:18) is individually screened for 22 h every 3 months until the age of 18 months. Data are automatically collected with 1 min intervals and analyzed either entirely or only for the dark (active) phase. Rodents not consuming >3 ml of water within a 24 h period are excluded from the analysis.

Light and Electron Immunohistochemistry: Rodents are deeply anesthetized with ketamine/xylazine (100/10 mg/kg, i.p.) and transcardially perfused with 4% paraformaldehyde in 0.1 m sodium cacodylate buffer, pH 7.4, followed by postfixation of the brains in the same fixative overnight. For light microscopical immunohistochemistry, 16 rodent brains are embedded in one gelatin block; up to 40 p.m thick coronal sections are freeze-cut and collected into 24 series (NeuroScience Associates). Free-floating staining is performed as previously described. Sections are incubated with the polyclonal 5830 antibody (1:15,000; ˜10 ng/ml), EM48 (1:300, MAB5374; Millipore Bioscience Research Reagents) or the polyclonal anti-calbindin D-28K antibody (1:50,000; Swant Swiss antibodies) followed by the respective secondary antibodies: biotinylated rabbit anti-sheep IgG antibody (1:1000, BA-6000; Vector Laboratories), biotinylated goat anti-mouse IgG antibody (1:1000, BA-9200; Vector Laboratories), or biotinylated goat anti-rabbit IgG antibody (1:1000, BA-1000; Vector Laboratories). Then sections are treated with an avidin—biotin—peroxidase complex (Vector Laboratories), and exposed to nickel-DAB-H202 (0.6% nickel sulfate, 0.01% DAB, and 0.001% hydrogen peroxide) until a suitable staining intensity develops. 5830 staining is amplified using a single round of biotinylated tyramine amplification before the final ABC step. Images are taken using an Axioplan 2 Microscope (Zeiss) with a digital camera (AxioCam MRm; Zeiss) and imaging acquisition software (AxioVision-6; Zeiss). Quantification is performed using ImageJ (NIH).

For electron microscopy BACHD and control rodent brains (13 and 16.5 months of age) are adjusted in Plexiglas frames according to the coordinates of a rodent brain atlas, embedded in 2% agarose, and cut into 3 mm coronal brain blocks. Blocks are cut into a series of 50 p.m vibrotome sections and immunostained with the monoclonal EM48 antibody (1:100, MAB5374; Millipore Bioscience Research Reagents). Immunostained sections are photodocumented for later detection of the reaction product and flat embedded in Araldite (Serva) as described previously. Ultrodenthin sections (90 nm) are contrasted with 5% aqueous uranyl acetate and lead citrodente (pH 12.0).

Quantitative assessment of morphological changes in the striosome compartment: Calbindin immunostaining of rodents at 6 months of age is used (TG5:TG9:WT=5:4:5) to perform a relative quantification of the striatal striosome compartment. Four striata of each rodent are measured in up to coronal 40-p.m-thick brain sections between bregma 1.44 and −0.24 mm. The striatum is outlined medially adjacent to the lateral ventricle, dorsolaterally below the corpus callosum, and ventrally by a line through the ventral tip of the ventricle as the region of interest (ROI) using ImageJ (NIH). The striosomal area is determined within the ROI by outlining the faintly stained calbindin areas.

[¹¹C]raclopride positron emission tomographic imaging: For longitudinal positron emission tomography (PET) experiments, transgenic BACHD and control rodents are imaged at 6, 12, and 18 months of age (n=6 of each genotype at each time point) using an Inveon dedicated small-animal PET scanner (Siemens Preclinical Solutions), yielding a spatial resolution of ˜1.3 mm in the reconstructed image. Conscious animals are lightly restrained and injected with 29.6 MBq [¹¹C]raclopride via one of the lateral tail veins. A 60 min dynamic PET scan is obtained immediately after tracer injection followed by a 15 min attenuation correction. During imaging, the animals are anesthetized with a mixture of isoflurane in oxygen. The animals are centered in the field of view of the PET scanner. Anesthesia is monitored by measuring respirodentory frequency, and the body temperodenture is kept at 37° C. by a heating pad underneath the animal. PET data are acquired in list mode; graphed in time frames of 4×60 s, 3×120 s, 7×300 s and 2×450 s, and reconstructed using a filtered back projection algorithm with a matrix size of 256×256 and a zoom factor of two. Images are analyzed using PMOD and AsiPro software (Siemens Preclinical Solutions). The PMOD image fusion software allowed for linear transformation and rotation to overlay the PET and magnetic resonance (MR) template images. The fusioned PET/MR images are analyzed to calculate specific ROIs in different brain areas with reference to the stereotactic brain atlas of Paxinos and Franklin (2006).

Statistical analysis: Standard two-way ANOVA (data not matched) and repeated-measures two-way ANOVA (repeated or matched data) are conducted to assess the effects of treatment. For data where only one time point is assessed (such as the footprint test and matrix/striosome analysis), one-way ANOVA is conducted to evaluate the effects of treatment, followed by Tukey's post hoc test for multiple comparisons. Data are presented as mean±SEM. Differences are considered significant if p<0.1.

Results: It is predicted that methods of the present technology will induce reversal of symptoms and/or pathologies of HD or delay the onset of symptoms and or pathologies of HD in the BACHD transgenic rodent model. These results will show that methods of the present technology are useful and effective for the prevention or treatment of HD.

### Human Clinical Trials

Human subjects diagnosed as having or predisposed to developing HD, and/or presently displaying one or more symptoms of HD and/or pathologies of HD, are recruited. Symptoms of HD include, but are not limited to, e.g., motor deficits, cognitive decline, and psychological disturbances.

Symptoms and/or pathologies of HD may be assessed with respect to both the severity of a subject's disease condition and the efficacy of prevention or treatment methods. For example, the subject may be assessed using evaluations accepted as relevant to HD.

Methods of Prevention or treatment: Subjects are administered methods of the present technology at a dosage and frequency commensurate with the stage and severity of disease. In some embodiments the method is administered once daily, once weekly, or once monthly. In some embodiments, the method is administered multiple times daily, weekly, or monthly.

To demonstrate methods of prevention or treatment in human subjects, subjects are administered methods of the present technology prior to or subsequent to the development of symptoms and/or pathologies or HD and assessed for reversal of symptoms/pathologies of HD, delay of the onset of symptoms/pathologies of HD, or attenuation of expected symptoms/pathologies.

Results: It is predicted that methods of the present technology will induce reversal of symptoms and/or pathologies of HD or delay the onset of symptoms and or pathologies in human subjects. These results will show that methods of the present technology are useful and effective for the prevention or treatment of HD.

### Example 3

### Methods and Devices of the Present Technology for the Prevention and Treatment of Parkinson's Disease and Related Disorders

This example demonstrates the use of methods and devices of the present technology in the prevention and treatment of Parkinson's Disease (PD) and related disorders in neurotoxic animal models, genetic animal models, and human subjects.

### Animal Models

Neurotoxic Animal Models: Neurotoxic PD models are generated by administering neurotoxic agents to animal subjects at a level and frequency sufficient to cause one or more symptoms and/or pathologies of PD. Animals used in the study can include rodents, primates, dogs, cats, etc. Dosage level and frequency of administration varies with and can be determined based on the particular agent and animal in use. Symptoms and/or pathologies of PD may be assessed with respect to both the efficacy of the neurotoxic agent (i.e. the severity of a subject's disease condition) and the efficacy of prevention and treatment methods.

Neurotoxic agents suitable for use in the example include, but are not limited to, 6-hydroxy dopamine (6-OHDA), 1-methyl-1,2,3,6-tetrahydropyridine (MPTP), Paraquat, Rotenone, reserpine, a-methyl-p-tyrosine, p-chloroamphetamine (PCA), methamphetamine, 3,4-methylenedioxymethamphetamine (MDMA), Fenfluramine, isoquinoline derivatives (e.g., 1,2,3,4-tetrahydroisoquinoline), and lipopolysaccharide (LPS) models.

Genetic Animal Models: Genetic PD models suitable for use in the example include, but are not limited to, subjects having mutations in a-synuclein, Leucine-rich repeat kinase 2 (LRKK2), PTEN-induced putative kinase 1 (PINK1), Parkin, Protein deglycase (Parkinson disease protein 7; DJ-1), ATPase 13A2 (ATP13A2), Sonic hedgehog (SHH), Nuclear receptor related 1 protein (Nurrl), Engrailed 1 (Enl), Pituitary homeobox 3 (Pitx3), c-rel-NFkB, Autophagy Related 7 (Atg7), Vesicular monoamine transporter 2 (VMAT2), and Mitochondrial transcription factor A (e.g. MitoPark mice).

Symptoms and/or pathologies of PD or related disorder may be assessed with respect to both the severity of a subject's disease condition and the efficacy of prevention and treatment methods. For example, the subject may be assessed using neurological evaluations relevant to PD or related disorders.

Animals and groups: Male nine-week-old C57BL/6mice (Orientbio Inc., Seongnam, Korea) weighing 20-23 g are housed in a Plexiglas cage (200 mm×320 mm×145 mm, 3 mice in each cage) at room temperature (22±2° C.) under a standard 12-h light/dark cycle with unlimited access to a standard laboratory diet (Orientbio Inc.) and water. The animals are handled in accordance with the current guidelines established in the National Institutes of Health Guide for the Care and Use of Laboratory Animals (NIH Publication No. 85-23, 1985). The physical condition of the mice is monitored every other day in the adaptation period and every day in the experimental period. Humane endpoints for the study are as follows: 1) weight loss more than 20%; 2) no food intake more than 3 days; 3) diarrhea more than 3 days; 4) severe tremor or motor dysfunction insufficient to evaluate subject behaviors. For immunostaining, mice are anesthetized with isoflurane and sacrificed by perfusion. Mice are randomly attached to three groups (n=9 at each group): a saline-injected group (Saline), a MPTP-injected group (MPTP) and a MPTP-injected plus one or more therapeutic methods of the current technology.

Neurotoxic agent injection: For neurotoxic agent models, agents are injected at an appropriate dose and frequency for the animal model and agent used. The following MPTP protocol is given as illustrative: all mice except those in the saline group are injected with MPTP-HC1 (20 mg/kg; Sigma, St. Louis, Mo., USA) intraperitoneally four times at 2 h intervals (total 80 mg/kg). Mice in the saline group are injected with vehicle (normal saline) on the same schedule.

Assessment of symptoms: Symptoms and behaviors associated with Parkinson's are measured and assessed. The following pole test is given as illustrative: mice (n=9 at each group) are mounted head-downwards near the top of a rough-surfaced wood pole (10 mm in diameter and 55 cm in height), and the time taken to reach the bottom of the pole is measured. The test is repeated three times at 30 s time intervals, after which behavioral change is evaluated according to the average of the three times. For neurotoxic animal models, the test is performed one day before neurotoxic agent injection (day 0) and 2 h after the last administration of therapy.

Immunohistochemistry: Mice are perfused with 4% paraformaldehyde dissolved in 0.1M phosphate buffer, and the brain quickly harvested, post-fixed in the 4% paraformaldehyde buffer for 48 hours, and immersed in 30% sucrose solution for storage at 4° C. prior to sectioning. Frozen sections are cut to a thickness of 35 p.m using a cryostat Leica CM3050S (Leica Microsystems, Wetzlar, Germany). The sections are incubated with 1% H202 in 0.05 M phosphate-buffered saline for 15 min, followed by 0.3% Triton X-100 and 3% normal blocking serum in PBS at room temperature for 1 h, then stained overnight at room temperature using an anti-tyrosine hydroxylase (TH, 1:500; Santa Cruz Biotechnology, Santa Cruz, Calif., USA) primary antibody. The next day, sections are incubated with Vectastain Elite ABC reagents (Vector Laboratories Inc., Burlingame, Calif.) at room temperature for 1 h, then incubated with a diaminobenzidine substrate kit (Vector Laboratories Inc.) for 5 min. The tissues are subsequently mounted on gelatin-coated slides, air-dried, dehydrated, and cover-slipped. Images are collected using an Axio Scope.A1 microscope (Zeiss, Germany) and an AxioCamICc3 camera (ZEISS). The survival of dopaminergic neurons in the SN is evaluated by the number of TH-positive neuronal cells. An independent observer not knowing the expected results manually counts TH-positive neurons bilaterally in five continuous SN sections, with cell counting confirmed three times to validate the data. The survival of dopaminergic neurons in ST is evaluated by the mean value of optical density in ST using an Image-Pro Plus 6.0 (Media Cybernetics, Silver Spring, Md., USA).

To demonstrate methods of prevention in neurotoxic animal models, subjects are administered methods of the present technology, including, for example, a combination therapy, simultaneous to or subsequent to the administration of neurotoxic agents and before the development of symptoms or pathologies of PD. To demonstrate methods of treatment in neurotoxic animal models, subjects are administered methods of the present technology subsequent to the administration of neurotoxic agents and subsequent to the development of symptoms or pathologies of PD. Symptoms and/or pathologies of PD may be assessed at predetermined time points.

To demonstrate methods of prevention and treatment in and genetic animal models, subjects are administered methods of the present technology prior to or subsequent to the development of symptoms and/or pathologies or PD or related disorders and assessed for reversal of symptoms/pathologies or attenuation of expected symptoms/pathologies.

Statistical analysis: All data is expressed as the mean±standard deviation and analyzed by one-way analysis of variance with the Neuman-Keuls post hoc test. All statistical testing is performed using Prism 5 for Windows (Graph Pad Software Inc., La Jolla, Calif., USA) with statistical significance set atp<0.05.

Results: It is predicted that methods of the present technology will induce reversal of symptoms and/or pathologies of PD and related disorders in animal models. These results will show that methods of the present technology are useful and effective for the prevention and treatment of such disorders.

### Human Clinical Trials

Human subjects diagnosed as having or suspected to have PD or a related disorder and presently displaying one or more symptoms and/or pathologies of PD or a related disorder, including, but not limited to tremor, rigidity, akinesia/bradykinesia, and postural instability, are recruited.

In some studies, subjects are diagnosed as having or suspected to have a sporadic PD or related disorder. In some studies, subjects are diagnosed as having or suspected to have a familial PD or related disorder. In some studies, subjects are diagnosed as having or suspected to have atypical parkinsonism or Parkinson-plus syndromes including, but not limited to multiple system atrophy (MSA), progressive supranuclear palsy (PSP), corticobasal degeneration (CBD), dementia with Lewy bodies (DLB), Pick's disease, olivopontocerebellar atrophy, and Shy-Drager syndrome. In some studies, the conditions are characterized by synucleinopathies. In some studies, the conditions are characterized by tauopathies.

Clinical studies are conducted in accordance with accepted practices, such as, for example, the protocol of van de Weijer, et al., BMC Neurology 16: 1-11 (2016).

Methods of Prevention and Treatment: Subjects are administered methods of the present technology at a dosage and frequency commensurate with the stage and severity of disease. In some embodiments the method is administered once daily, once weekly, or once monthly. In some embodiments, the method is administered multiple times daily, weekly, or monthly.

To demonstrate methods of prevention and treatment in humans, subjects are administered methods of the present technology prior to or subsequent to the development of symptoms and/or pathologies or PD or related disorders and assessed for reversal of symptoms/pathologies or attenuation of expected symptoms/pathologies.

Results: It is predicted that methods of the present technology will induce reversal of symptoms and/or pathologies of PD and related disorders in human subjects. These results will show that methods of the present technology are useful and effective for the prevention and treatment of such disorders.

### Example 4

### Methods and Devices of the Present Technology for the Prevention and Treatment of Multiple Sclerosis

This example demonstrates the use of methods and devices of the present technology in the prevention and treatment of multiple sclerosis (MS) utilizing animal models and human subjects.

### Animal Models

Animal models suitable for use in this example include the murine model of chronic-relapsing experimental autoimmune encephalomyelitis (EAE) and in human subjects. Methods are performed in accordance with the methods of Wujek, J. R., et al., J. Neuropathol. Exp. Neurol. 61(1):21-32 (2002) and Yu, M., et al., J. Neuroimmunol. 64:91-100 (1996).

Female SWXJ (H-2q,$) mice immunized with the p139-151 peptide of myelin proteolipid protein (PLP) develop relapsing-remitting chronic EAE characterized initially by intermittent attacks of reversible neurological impairment, with a late plateau of sustained functional disability. The predominant site of inflammatory tissue injury in this model is the spinal cord and the clinical rating scale used in EAE (as in MS) emphasizes spinal cord function. Therefore, the clinical, histological, and temporal pattern of disease in this EAE model mimic the most common profile observed in MS. The relapsing-remitting EAE mouse model is useful to evaluate efficacy of neuroprotective therapies.

To demonstrate methods of prevention in EAE animal model, subjects are administered methods of the present technology simultaneous to or subsequent to intermittent attacks of reversible neurological impairment and before the development of symptoms or pathologies of MS. To demonstrate methods of treatment in EAE animal model, subjects are administered methods of the present technology subsequent to intermittent attacks of reversible neurological impairment and subsequent to the development of symptoms or pathologies of MS. Symptoms and/or pathologies of MS may be assessed at predetermined time points.

Induction of EAE: SWXJ (H-2q,$) mice are produced by mating SWR/J (H-2q) females with SJL/J (H-2s) males. Animals are handled and maintained in accordance with approved guidelines.

To induce chronic-relapsing EAE, adult female mice are injected subcutaneously on Day 0 with a mixture of 100 nanomoles PLP peptide p139-151 and 400 micrograms Mycobacterium tuberculosis H37RA in complete Freund's adjuvant. The mice are also injected intravenously, on Day 0 and Day 3, with 2×109 to 3×109 Bordetellapertussis bacilli (Michigan Department of Public Health, Lansing, Mich.). Control mice receive the same administrations, but without the PLP peptide.

Monitoring Clinical EAE: Mice are weighed and examined daily for neurological signs according to previously published criteria: 0=no observable symptoms; 1=flaccid tail; 2=poor righting reflex; 3=clumsy gait; 4=limb paralysis; 5=moribund. Mice are killed 5 days after the first neurological attack, defined as a concomitant increase in clinical score and loss of body weight, or at the chronic stage of EAE (3 months post-immunization).

The peak severity of acute EAE is defined as the highest score achieved by each mouse during the first 5 days of the first attack. It is predicted that, generally, this score is maintained for at least 2 consecutive days. Clinical severity during the plateau phase of chronic non-remitting EAE is established as a disability score that did not change for 1 week, at least 45 days post-immunization.

Light Microscopy and Immunohistochemistry: Mice are deeply anesthetized and perfused through the heart with 4% paraformaldehyde in 0.08 M phosphate buffer. The cervical and lumbar spinal cord segments are removed, post-fixed, and cryoprotected in 20% glycerol. Free-floating cross-sections (30-um-thick) are cut, placed in cryostorage solution (1% polyvinylpyrolidone-40, 30% ethylene glycol, and 30% sucrose in 0.2 M phosphate buffer) and stored at −22° C. For immunostaining, sections are rinsed with phosphate-buffered saline, incubated in tris-buffered saline with 0.25% Triton X-100 plus 0.3% hydrogen peroxide, and incubated with a primary antibody. The antibodies are a rabbit polyclonal antibody against the 200 kDa neurofilament protein (Serotec, Raleigh, N.C.; AHP245; diluted 5 1:10,000); a rat monoclonal antibody against CD45 (Serotec; MCA1388; diluted 5 1:8,000); a rabbit polyclonal antibody against CD3 (Dako, Carpinteria, Calif.; A0452; diluted 1:4,000); or a rat monoclonal antibody against proteolipid protein (Agmed, Bedford, Mass.; diluted 1:8,000). Sections are rinsed, incubated in biotinylated secondary antibodies (Vector Labs, Burlingame, Calif.), rinsed, incubated in an avidin-biotin peroxidase complex (Vector Labs), and visualized with a nickel-intensified diaminobenzidine reaction. Tissue sections are mounted on microscope slides and coverslipped.

Electron Microscopy: Mice are deeply anesthetized and perfused with 2.5% (wt/vol) glutaraldehyde and 4% (wt/vol) paraformaldehyde in 0.08 M phosphate buffer. Spinal cord segments are dissected out, post-fixed, and embedded in Epon. Ultrathin sections of white matter are cut in the transverse plane, mounted on Formvar coated grids, and photographed in a Philips CM100 electron microscope.

Morphometry: To determine the extent of inflammation in control and EAE spinal cord, the level of CD45 immunoreactivity (microglia, macrophages, monocytes, and lymphocytes) is quantified. Images at low magnification (53 objective lens) of whole spinal cord are digitally photographed using a Leica DMR microscope fitted with an Optronics Magna Fire CCD color video camera and image acquisition system. Digital images are captured (using Adobe Photoshop 5.0 software; Adobe Systems Inc., San Jose, CA) and coded. Spinal cord area occupied by CD45 immunoreactivity is determined by measuring number of pixels above a set threshold value. Spinal cord area is measured (total pixels within spinal cord) and the percent area of CD45 immunoreactivity is calculated. To determine lymphocyte density, the numbers of lymphocytes are measured in white matter of spinal cord sections stained for CD3. With an ocular reticule, CD3-positive cells are counted at high magnification (1003 objective and 103 ocular lens). For each animal, a total area of 0.9 mm2 is analyzed and values are calculated as cells per mm2. To determine the extent of axon loss, axons are counted in anti-eurofilament stained spinal cord sections of EAE and control mice. Digital images at high magnification are captured in selected areas, as described above. Axon counts are made in a blinded fashion, using NIH Image computer software (version 1.61). Because axon density varied among the different spinal cord areas, the axonal number in each area of EAE spinal cord is expressed as a percent of mean control values in the same area. Since inflammatory tissue swelling is prominent in EAE, spinal cord areas are measured and used to normalize axonal densities.

Statistical Analysis: Data are analyzed with the Student t-test. Relationships between symptomatic attacks, clinical scores, and pathological alterations are evaluated by regression analysis. Specifically, the number of attacks experienced by each mouse is plotted against its final clinical score and extent of axonal loss. The correlation coefficient ® and the significance level (p) for these 2 variables are calculated using the Spearman rank correlation test.

Results: It is predicted that methods of the present technology will induce reversal of symptoms and/or pathologies in MS animal models. These results will show that methods of the present technology are useful and effective for the prevention and treatment of MS.

### Human Clinical Trials

Human subjects diagnosed as having or suspected to have MS and presently displaying one or more symptoms and/or pathologies of MS are recruited. Symptoms and/or pathologies of MS may be assessed with respect to both the severity of a subject's disease condition and the efficacy of prevention and treatment methods. For example, the subject may be assessed using evaluations generally accepted as relevant to MS.

Methods of Prevention and Treatment: Subjects are administered methods of the present technology at a dosage and frequency commensurate with the stage and severity of disease. In some embodiments the method is administered once daily, once weekly, or once monthly. In some embodiments, the method is administered multiple times daily, weekly, or monthly.

To demonstrate methods of prevention and treatment in human subjects, subjects are administered methods of the present technology prior to or subsequent to the development of symptoms and/or pathologies or MS and assessed for reversal of symptoms/pathologies or attenuation of expected symptoms/pathologies.

Results: It is predicted that methods of the present technology will induce reversal of symptoms and/or pathologies in MS human subjects or increase the efficacy of pharmacological agents used to treat MS. These results will show that methods of the present technology are useful and effective for the prevention and treatment of MS.

1. A method for reducing a rate of brain atrophy in one or more areas of the brain of a subject, said method comprising administering a non-invasive stimulus to said subject to induce synchronized gamma oscillations in a brain region of said subject, thereby reducing said rate of brain atrophy in said brain region of said subject.

2. The method of claim 1, wherein said non-invasive stimulus comprises one or more stimulation waveforms.

3. The method of claim 2, wherein said one or more stimulation waveforms comprise visual stimulation waveforms, auditory stimulation waveforms, haptic stimulation waveforms, mechanical stimulation waveforms, or a combination thereof.

4. The method of claim 3, wherein said one or more stimulation waveforms have synchronous phases.

5. The method of claim 2, wherein said one or more stimulation waveforms comprise a first stimulation waveform and a second stimulation waveform.

6. (canceled)

7. The method of claim 2, wherein said one or more stimulation waveforms comprise auditory stimulation waveforms.

8. The method of claim 7, wherein said auditory stimulation waveform comprises an up-chirp.

9. The method of claim 7, wherein said auditory stimulation waveform comprises a down-chirp.

10. The method of claim 7, wherein said auditory stimulation waveform comprises white noise.

11. The method of claim 2, wherein said one or more stimulation waveforms have varying pulse rate intervals.

12. The method of claim 2, wherein said one or more stimulation waveforms have constant pulse rate intervals.

13. The method of claim 5, wherein said first stimulation waveform comprises a square wave function.

14. The method of claim 5, wherein said first stimulation waveform comprises a sinusoidal wave function.

15. The method of claim 5, wherein said second stimulation waveform comprises a square wave function.

16. The method of claim 5, wherein said second stimulation waveform comprises a sinusoidal wave function.

17. The method of claim 1, wherein administering said non-invasive stimulus comprises administering said non-invasive stimulus for a first time duration.

18. The method of claim 17, further comprising measuring a response of said subject to said non-invasive stimulus during a second time duration.

19. The method of claim 18, wherein said second first duration and said second time duration are separated by a third time duration.

20. The method of claim 1, wherein said non-invasive stimulus is delivered through a wearable device.

21. The method of claim 2, wherein said one or more stimulation waveforms vary in frequency.

22. The method of claim 2, wherein said one or more stimulation waveforms comprise a constant frequency.

23-29. (canceled)

30. The method of claim 1, wherein said brain region comprises a visual cortex, a somatosensory cortex, an insular cortex, or any combination thereof.

31. The method of claim 1, wherein reducing said rate of brain atrophy comprises reducing a rate of decrease in brain volume.

32. The method of claim 31, wherein said rate of decrease in brain volume comprises from about 0.3 cm³ per month to about 2 cm³ per month.

33. The method of claim 31, wherein said rate of decrease in brain volume comprises from about 0.3 cm³ per year to about 2 cm³ per year.

34. The method of claim 31, wherein said rate of decrease in brain volume comprises a rate of decrease in hippocampal volume, lateral lobe volume, lateral ventricle volume, temporal lobe volume, occipital lobe volume, temporal cortex thickness, occipital cortex thickness, or a combination thereof.

35.-52. (canceled)

53. A method of reducing one or more symptoms or conditions associated with brain atrophy, said method comprising: (a) identifying a subject experiencing brain atrophy; and (b) administering non-invasive stimulus to a subject, said non-invasive sensory stimulus causing synchronization of one or more brainwaves, thereby reducing said one or more symptoms associated with brain atrophy.

54. The method of claim 53, wherein said one or more symptoms or conditions comprise loss of neurons, memory loss, blurred vision, aphasia, impaired balance, paralysis, decreases in cortical volume, increases in CSF volume, loss of motor control, difficulty speaking, difficulty with reading comprehension, decrease in gray matter volume, decreases in white matter volume, decrease in neuronal size, loss of neuronal cytoplasmic proteins, or any combination thereof.

55.-69. (canceled)
